(19) United States
(12) Patent Application Publication
(10) Pub. No.: US 2013/0280764 A1
(43) Pub. Date: Oct. 24, 2013

(54) METHOD OF IMPROVING THE ACTIVITY OF CELLULASE ENZYME MIXTURES IN THE SACCHARIFICATION (LIGNO)CELLULOSIC MATERIAL

(71) Applicants: Mark EMALFARB, Jupiter, FL (US); Arkady SINITSYN, Moscow (RU); Richard JUNDZIL, Jupiter, FL (US); Jan WERY, Gorssel (NL); Jacob VISSER, Wageningen (NL); Rob JOOSTEN, Wageningen (NL); Martijn KOETSIER, Odijk (NL)

(72) Inventors: Mark EMALFARB, Jupiter, FL (US); Arkady SINITSYN, Moscow (RU); Richard JUNDZIL, Jupiter, FL (US); Jan WERY, Gorssel (NL); Jacob VISSER, Wageningen (NL); Rob JOOSTEN, Wageningen (NL); Martijn KOETSIER, Odijk (NL)

(73) Assignee: DYADIC INTERNATIONAL (USA) LTD., Jupiter, FL (US)

(21) Appl. No.: 13/866,620

(22) Filed: Apr. 19, 2013

Related U.S. Application Data
(60) Provisional application No. 61/635,850, filed on Apr. 19, 2012.

(51) Int. Cl. C12N 9/24; C12P 19/14; C12N 15/80

(52) U.S. Cl. CPC C12N 9/2437; C12N 15/80; C12P 19/14
USPC 435/99; 435/254.11; 435/254.4; 435/254.5; 435/254.8; 435/254.3; 435/254.7; 435/254.6; 435/209; 435/196

(57) ABSTRACT

The present invention relates to modified filamentous fungal organisms having improved activity profiles with respect to the conversion of complex carbohydrates into simple sugars from cellulosic materials, including fungal organisms belonging to a genus selected from the group consisting of: Chrysosporium, Thielavia, Talaromyces, Thermomyces, Thermoascus, Neurospora, Aureobasidium, Filibasidium, Piromyces, Corynascus, Cryplococcus, Acremonium, Tolypocladium, Scytalidium, Schizophyllum, Sporotrichum, Penicillium, Gibberella, Myceliophthora, Mucor, Aspergillus, Fusarium, Humicola, Trichoderma, and Talaromyces, plus anamorphs and teleomorphs thereof. Filamentous fungal organisms having improved activity profiles are obtained by modifying genes encoding enzymes involved in the production of cellobionolactone, cellobionic acid, gluconolactone, gluconic acid, and related products, by a variety of mutagenic methods, resulting in nucleotide substitutions, insertions, and deletions, increasing the level of saccharification in enzyme mixtures obtained from the modified organisms.

### CROSS REFERENCE TO RELATED APPLICATIONS

The pending application claims priority under 35 U.S.C. §119(e) to U.S. Provisional Patent Application No. 61/635,850, filed on Apr. 19, 2012, the disclosure of which is expressly incorporated herein by reference.

This application expressly incorporates by reference the contents of the following United States patents and patent applications: U.S. Pat. No. 5,811,381, issued Sep. 22, 1998; U.S. Pat. No. 6,015,707, issued Jan. 18, 2000; U.S. Pat. No. 6,573,086, issued Jun. 3, 2003; U.S. Pat. No. 7,122,330, issued Oct. 17, 2006; U.S. Pat. No. 7,399,627, issued Jul. 15, 2008; U.S. Pat. No. 7,794,962, issued Sep. 14, 2010; U.S. Pat. No. 7,883,872, issued Feb. 8, 2011; U.S. Pat. No. 7,892,812, issued Feb. 22, 2011; U.S. Pat. No. 7,906,309, issued Mar. 15, 2011; U.S. Pat. No. 7,923,236, issued Apr. 12, 2011; U.S. Patent Publication No. 2008-0076159, published Mar. 27, 2008; U.S. Publication No. 2008-0194005, published Aug. 14, 2008; U.S. Publication No. 2009-0099079, published Apr. 16, 2009; U.S. Publication No. 2011-0047656, published Feb. 24, 2011; U.S. Publication No. 2011-0045546, published Feb. 24, 2011; U.S. Publication No. 2011-0237485, published Sep. 29, 2011; U.S. Publication No. 2011-0287135, published Nov. 24, 2011; U.S. Publication No. 2012-0030839, published Feb. 2, 2012; U.S. Publication No. 2012-0030838, published Feb. 2, 2012; U.S. Publication No. 2012-0036599, published Feb. 9, 2012; U.S. patent application Ser. No. 13/046,772, filed Mar. 14, 2011; and U.S. patent application Ser. No. 13/138,661, filed Sep. 16, 2011.

The content of all other patents, patent applications, publications, articles, or literature cited herein are expressly incorporated by reference, as if written herein.

### INCORPORATION-BY-REFERENCE OF A SEQUENCE LISTING

The sequence listing contained in the file “124702_{—}0275_US_ST25.txt”, created on 2013 Apr. 19, modified on 2013 Apr. 19, file size 152,459 bytes, is incorporated by reference in its entirety herein.

### FIELD OF THE INVENTION

The present invention relates to modified filamentous fungal organisms having improved activity profiles with respect to the conversion of complex carbohydrates into simple sugars from cellulosic materials, including fungal organisms belonging to a genus selected from the group consisting of: Chrysosporium, Thielavia, Talaromyces, Thermomyces, Thermoascus, Neurospora, Aureobasidium, Filibasidium, Piromyces, Corynascus, Cryplococcus, Acremonium, Tolypocladium, Scytalidium, Schizophyllum, Sporotrichum, Penicillium, Gibberella, Myceliophthora, Mucor, Aspergillus, Fusarium, Humicola, Trichoderma, and Talaromyces, plus anamorphs and teleomorphs thereof. Filamentous fungal organisms having improved activity profiles are obtained by modifying genes encoding enzymes involved in the production of cellobionolactone, cellobionic acid, gluconolactone, gluconic acid, and related products, by a variety of mutagenic methods, resulting in nucleotide substitutions, insertions, and deletions, increasing the level of saccharification in enzyme mixtures obtained from the modified organisms.

### BACKGROUND OF THE INVENTION

Enzymes used in industrial applications are often produced as mixtures of enzymes through fermentation. Classical microbiology relied upon a variety of methods of mutating microorganisms and selecting mutants having desired properties. Many of these methods relied upon chance and selective pressure to produce organisms having desired phenotypes. Modern methods involving genetic engineering often allow the insertion of desirable genes into a microorganism, or the inactivation of undesirable genes by mutation, through the introduction of mutations (e.g., one or more nucleotide substitutions, insertions, or deletions, or a combination thereof) when such genes are known. The levels of expression of products of interest can also be increased or decreased, if appropriate genetic elements are available for manipulation under different environmental conditions. Key tools include systems for transforming microorganisms with genetic material that can easily be modified, such as plasmids or viruses, and related types of DNA constructs which can be designed to mutate, disrupt, or delete specific genes, or to over-express genes and gene products of interest. Modern methods of altering genetic material can also be combined with classical methods involving random mutagenesis to produce microorganisms having desirable properties.

The modification, isolation, and characterization of microorganisms which produce desirable enzyme mixtures for the optimal hydrolysis of (ligno-) cellulosic materials is quite challenging. The initial step often involves the identification of genes which encode enzymes having activities that are beneficial or detrimental for hydrolysis of (ligno-) cellulosic materials. Methods which modify the genetic material of microorganisms which result in higher or lower expression, or complete inactivation of such genes is often a major challenge.

Elimination of detrimental enzymes which form cellobionolactone/cellobionic acid and/or gluconolactone/gluconic acid which are produced at the expense of cellobiose and/or glucose is desired in enzyme mixtures optimized for (ligno-) cellulose hydrolysis. Cellobionolactone/cellobionic acid and/or gluconolactone/gluconic acid, when present, have an adverse effect on the production of cellobiose and/or glucose. Omission of cellobionolactone/cellobionic acid and/or gluconolactone/gluconic acid, therefore, often results in higher cellobiose and/or glucose yields.

Enzymes causing the formation of cellobionolactone/cellobionic acid and/or gluconolactone/gluconic acid include: cellobiose dehydrogenases (CDH) glucooligosaccharide dehydrogenases, glucose dehydrogenases, glucooligosaccharide oxidases, cellobiose oxidases, glucose oxidases and enzymes that belong to Glycoside Hydrolase Family 61 (GH61) (also referred to as copper-dependent polysaccharide monooxygenases or polypeptides having cellulolytic enhancing activity).

Elimination of cellobionolactone/cellobionic acid and/or gluconolactone/gluconic acid forming enzymes also increases the activity of the remaining (hemi)cellulase enzymes by reducing inhibition by cellobionolactone/cellobionic acid and/or gluconolactone/gluconic acid of the remaining (hemi)cellulases.

Elimination of cellobionolactone/cellobionic acid and/or gluconolactone/gluconic acid producing enzymes also reduces acidification of the reaction which may improve the activity of the enzyme mixture and reduces the amount of base necessary to maintain pH.

Genes encoding CDHs are present (and naturally expressed) in C1. Genes encoding putative CDHs have been identified in numerous fungal species including species of Aspergillus and Talaromyces (See, Harreither W, Sygmund C, Augustin M, Narciso M, Rabinovich M L, Gorton L, Haltrich D, Ludwig R. (2011) Catalytic properties and classification of cellobiose dehydrogenases from ascomycetes. Appl Environ Microbiol. 77(5):1804-15) which describes the classification and occurrence of CDHs in ascomycetous fungi; and see WO2010/080532 METHODS FOR INCREASING HYDROLYSIS OF CELLULOSIC MATERIAL IN THE PRESENCE OF CELLOBIOSE DEHYDROGENASE in which cellobiose dehydrogenase is claimed in the degradation or conversion of cellulosic material. Contrary to the present invention, the examples in this reference show that adding CDH increases degradation of cellulosic material.

No gene encoding a CDH has been identified in the genome of Trichoderma reesei; and CDH activity has not been identified in Talaromyces emersonii. However, almost all fungal species are likely to contain at least one of the enzyme activities leading to formation of cellobionolactone/cellobionic acid and/or gluconolactone/gluconic acid.

Copper-dependent polysaccharide monooxygenases (or GH61 or polypeptides having cellulolytic enhancing activity) have been demonstrated to increase the activity a cellobiose dehydrogenase (see Phillips C M, Beeson W T, Cate J H, Marietta M A. (2011) Cellobiose dehydrogenase and a copper-dependent polysaccharide monooxygenase potentiate cellulose degradation by Neurospora crassa. ACS Chem Biol. 6(12):1399-406. This paper shows that a copper-dependent polysaccharide monooxygenases (or GH61s or polypeptides having cellulolytic enhancing activity) can increase the activity of a CDH. The advantage of a CDH knock-out is therefore more pronounced in enzyme mixtures also containing these copper-dependent polysaccharide monooxygenases (or GH61 or polypeptides having cellulolytic enhancing activity).

Analogous to the oxidation of glucose, cellobiose and glucoologosaccharides the oxidation of xylose, xylobiose and xylooligosaccharides may have a similar effect on the performance of (hemi)cellulases. Elimination of these enzyme activities may therefore be beneficial.

Similarly, enzymatic activities resulting in the oxidation of chitin(oligos), chitosan(oligos) and glucosamine; (gluco)mannan(oligos), galacto(gluco)mannan(oligos) and mannose; arabinan(oligos) and arabinose; and galactan and galactose may have a similar effect on the performance of (hemi)cellulases. Elimination of these enzyme activities may therefore be beneficial.

### SUMMARY OF THE INVENTION

The invention is directed to a modified fungus comprising one or more genes encoding enzymes having one or more cellulase or hemicellulase activities; wherein said fungus comprises one or more modified genes encoding enzymes responsible for the production of one or more products selected from cellobionolactone, cellobionic acid, gluconolactone, and gluconic acid, wherein the level of expression of said modified genes or the level of activity of modified enzymes encoded by said modified genes is reduced or eliminated compared to the endogenous level of expression or activity in a parent fungus lacking one or more of said modified genes.

The present invention is also directed to a composition for the degradation and saccharification of (ligno)cellulosic materials comprising a mixture of enzymes obtained from a modified fungus, wherein said composition has one or more enzymes having cellulase or hemicellulase activities, and lacks or has reduced levels or activities of one or more enzymes responsible for the production of one or more products selected from the group consisting of cellobionolactone, cellobionic acid, gluconolactone, and gluconic acid; wherein production of glucose with said composition in the presence of (ligno)cellulosic materials is enhanced above the endogenous level of glucose produced with a composition which has normal levels or activities of one or more enzymes responsible for the production of one or more products selected from cellobionolactone, cellobionic acid, gluconolactone, and gluconic acid.

The present invention is also directed to a method of increasing saccharification of cellulosic materials comprising: treating the cellulosic material with an enzyme composition comprising enzymes having one or more cellulase or hemicellulase activities, wherein the enzyme composition is obtained from a modified fungus comprising one or more modified genes encoding enzymes responsible for the production of one or more products selected from cellobionolactone, cellobionic acid, gluconolactone, and gluconic acid; wherein the level of expression of said modified genes is eliminated or reduced or the level of activity of modified enzymes encoded by modified genes is reduced or eliminated, compared to the endogenous level of expression or activity in a parent fungus lacking one or more of said modified genes.

The present invention relates to novel combinations of enzymes or enzyme mixtures and novel methods for generating fungal strains by gene deletion, disruption, mutation, classical or other screening methods, or otherwise producing the same. The invention relates to the development of fungal strains that produce these enzyme mixtures, lacking enzymatic activities or with reduced selected enzymatic activities produced by wild-type or mutant fungal strains, and preferably originating from the genera of Myceliophthora, preferably of the genus of Myceliophthora thermophila, and most preferably from the wild-type or mutants of the Myceliophthora thermophila strains deposited at the All-Russian Collection of Microorganisms of Russian Academy of Sciences (VKM), Bakhurhina St. 8, Moscow, Russia, 113184, under the terms of the Budapest Treaty on the International Regulation of the Deposit of Microorganisms for the Purposes of Patent Procedure on Aug. 29, 1996, as Chrysosporium lucknowense Garg 27K, VKM F-3500 D or at the Centraal Bureau voor Schimmelcultures (CBS), Uppsalalaan 8, 3584 CT Utrecht, The Netherlands for the purposes of Patent Procedure on Dec. 5, 2007. For example, Strain C1 was mutagenized by subjecting it to ultraviolet light to generate strain UV13-6 (Accession No. VKM F-3632 D). This strain was subsequently further mutated with N-methyl-N′-nitro-N-nitrosoguanidine to generate strain NG7C-19 (Accession No. VKM F-3633 D). This latter strain in turn was subjected to mutation by ultraviolet light, resulting in strain UV18-25 (Accession No. VKM F-3631 D). This strain in turn was again subjected to mutation by ultraviolet light, resulting in strain W1L (Accession No. CBS122189), which was subsequently subjected to mutation by ultraviolet light, resulting in strain W1L#100L (Accession No. CBS122190). More specifically, this invention relates to the reduction or elimination of undesired enzymatic activities from the enzyme combinations or enzyme mixtures. More specifically, this invention relates to the reduction or elimination of enzymatic activities causing the formation of cellobionolactone/cellobionic acid and/or gluconolactone/gluconic acid. Enzymes causing the formation of cellobionolactone/cellobionic acid and/or gluconolactone/gluconic acid include: cellobiose dehydrogenase (CDH), but may also include: glucooligosaccharide dehydrogenase, glucose dehydrogenase, glucooligosaccharide oxidase, cellobiose oxidase, glucose oxidase and copper-dependent polysaccharide monooxygenases (or GH61 or polypeptides having cellulolytic enhancing activity).

This invention also relates to novel methods for generating fungal strains that are lacking functional genes encoding enzymes causing the formation of cellobionolactone/cellobionic acid and/or gluconolactone/gluconic acid e.g., by gene deletion, gene disruption, or mutation.

This invention also relates to methods for generating fungal strains that are lacking or impaired in expression of genes encoding enzymes causing the formation of cellobionolactone/cellobionic acid and/or gluconolactone/gluconic acid, e.g., by deletion, disruption or mutation of gene expression regulatory sequences such as promoter sequences, terminator sequences, promoter activating sequences and sequences encoding transcription factors.

This invention also relates to methods for generating fungal strains that produce enzyme mixtures impaired in enzymatic activities causing the formation of cellobionolactone/cellobionic acid and/or gluconolactone/gluconic acid by random or site-directed mutation of the genes encoding the enzymes causing the formation of cellobionolactone/cellobionic acid and/or gluconolactone/gluconic acid.

This invention also relates to methods for generating fungal strains obtained by random mutagenesis or otherwise which create strains producing enzyme mixtures lacking or impaired in enzymatic activities causing the formation of cellobionolactone/cellobionic acid and/or gluconolactone/gluconic acid.

This invention also relates to the methods for producing enzyme mixtures lacking or impaired in enzymatic activities causing the formation of cellobionolactone/cellobionic acid and/or gluconolactone/gluconic acid by gene silencing.

This invention also relates to the methods for generating enzyme mixtures lacking or impaired in enzymatic activities causing the formation of cellobionolactone/cellobionic acid and/or gluconolactone/gluconic acid by inactivation, inhibition or removal of the enzymes causing the formation of cellobionolactone/cellobionic acid and/or gluconolactone/gluconic acid.

This invention also relates to the use of enzyme mixtures lacking or impaired in enzymatic activities causing the formation of cellobionolactone/cellobionic acid and/or gluconolactone/gluconic acid.

This invention also relates to screening for mesophilic, and thermophilic fungal strains naturally producing enzyme mixtures lacking or impaired in enzymatic activities causing the formation of cellobionolactone/cellobionic acid and/or gluconolactone/gluconic acid.

The invention also relates to a method to degrade lignocellulosic biomass or cellulosic substrates. The invention also relates to converting lignocellulosic biomass or (hemi)cellulosic substrates into fermentable sugars with enzymes that degrade lignocellulosic, (hemi)cellulosic, and even more complex plant cell wall material. The invention also relates to a method to release cellular contents by effecting degradation of the cell walls. The invention also relates to methods of using the novel enzymes compositions in a variety of commercial processes, such as washing or treating of clothing or fabrics, detergent processes, animal feed, food, baking, beverage, biofuel, starch preparation, liquefaction, biorefining, deinking and biobleaching of paper and pulp, oil and waste dispersing, and treatment of waste streams.

### BRIEF DESCRIPTION OF THE FIGURES

FIG. 1 shows the inhibition of M. thermophila C1 BGl1 by gluconolactone.

FIG. 2A shows the absence of CDH1 in a CDH1 knock-out strain.

FIG. 2B shows the absence of CDH2 in a CDH2 knock-out strain.

FIG. 3 shows the CDH activity as determined for enzyme mixtures produced by the ancestor strain, by the cdh1-gene disruption strain and by the cdh1/cdh2-gene disruption strain.

FIG. 4A shows the results of pretreated corn stover (PCS, 10%) saccharifications.

FIG. 4B shows the results of pretreated corn stover (PCS, 10%) saccharifications.

### DETAILED DESCRIPTION OF THE INVENTION

The present invention relates generally to the elimination or reduction of enzymes that play a role in reduction-oxidation reactions. In particular, the present invention relates to enzymes from a filamentous fungal strain denoted herein as C1 (Accession No. VKM F-3500 D), and methods of producing and using novel enzyme combinations lacking or impaired in the enzymes. The present invention relates generally to proteins that play a role in reduction-oxidation reactions. The invention also provides compositions that include at least one of the enzymes described herein for uses including, but not limited to, the degradation/modification of lignin or (hemi-) cellulose. The invention stems, in part, from the discovery that the elimination or reduction of enzymatic activities which lead to the production of cellobionolactone/cellobionic acid and/or gluconolactone/gluconic acid increase the activity of enzymes produced by the C1 fungus that exhibit high activity toward plant biomass.

The present invention also provides methods and compositions for aiding in the conversion of plant biomass to fermentable sugars that can, in turn, be converted to useful products. Such products may include, without limitation, metabolites, and biofuels. The methods include methods for degrading lignin and (ligno)cellulosic material using enzyme mixtures to liberate sugars. The compositions of the invention include enzyme combinations that break down lignin and (ligno)cellulose.

The invention is directed to a modified fungus comprising one or more genes encoding enzymes having one or more cellulase or hemicellulase activities; wherein said fungus comprises one or more modified genes encoding enzymes responsible for the production of one or more products selected from cellobionolactone, cellobionic acid, gluconolactone, and gluconic acid; wherein the level of expression of said modified genes or the level of activity of modified enzymes encoded by said modified genes is reduced or eliminated compared to the endogenous level of expression or activity in a parent fungus lacking one or more of said modified genes.

One aspect of the invention is directed to a modified fungus wherein said fungus is a filamentous fungus from a genus or genus and species selected from the group consisting of Chrysosporium, Thielavia, Talaromyces, Thermomyces, Thermoascus, Neurospora, Aureobasidium, Filibasidium, Piromyces, Corynascus, Cryplococcus, Acremonium, Tolypocladium, Scytalidium, Schizophyllum, Sporotrichum, Penicillium, Gibberella, Myceliophthora, Mucor, Aspergillus, Fusarium, Humicola, and Trichoderma, and Talaromyces emersonii, plus anamorphs and teleomorphs, and derivatives thereof. Another aspect is directed to a modified fungus wherein said filamentous fungus is Myceliophthora thermophila, including the strain designated Myceliophthora thermophila C1, and derivatives designated Garg 27K (Accession No. VKM F-3500 D); UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190). A preferred strain is the filamentous fungus is UV18-25 (Accession No. VKM F-3631 D).

Another aspect of the invention is directed to a modified fungus, comprising one or more modified genes encoding a cellulose cellobiose dehydrogenase, including those with a modified cdh gene, such as the cdh1 and cdh2 genes. Related aspects include a modified fungus wherein the cdh1 gene was removed or disrupted by removing or replacing all or part of the cdh1 gene with a gene encoding a selection marker. Other aspects include a modified fungus wherein the cdh1 gene was disrupted by replacing a part of the cdh1 gene, which may be with a gene encoding a selectable marker, exemplified by the AmdS selectable marker. Related aspects include a modified fungus comprising a modified cdh2 gene, and a modified fungus comprising a modified cdh1 and a modified cdh2 gene. Preferred strains of modified fungus include Garg 27K (Accession No. VKM F-3500 D); UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190), most preferably is UV18-25 (Accession No. VKM F-3631 D).

Another aspect of the invention relates to a modified fungus wherein the level of expression of said modified genes or the level of activity of modified enzymes is reduced or eliminated by modifying the coding sequence of one or more genes encoding said enzymes.

Another aspect of the invention relates to a modified fungus wherein the level of expression of said modified genes or the level of activity of modified enzymes is reduced or eliminated by modifying the noncoding sequence of one or more genes encoding said enzymes.

Another aspect of the invention relates to a modified fungus wherein the level of expression of said modified genes or the level of activity of modified enzymes is reduced or eliminated by introduction of one or more point insertions or deletions into the non-coding sequence of one or more genes encoding said enzymes.

Another aspect of the invention relates to a modified fungus wherein the level of expression of said modified genes or the level of activity of modified enzymes is reduced or eliminated by introduction of one or more point mutations, insertions, or deletions into the coding sequence of one or more genes encoding said enzymes.

Another aspect of the invention relates to a modified fungus wherein the level of expression of said modified genes or the level of activity of modified enzymes is reduced or eliminated from about 50% to about 100%, at least 75%, or at least 90% compared to the endogenous level of expression or activity in a parent fungus lacking one or more of said modified genes.

Another aspect of the invention relates to a modified fungus wherein the level of activity of an enzyme causing the formation of cellobionolactone or cellobionic acid is reduced from about 50% to about 100%, at least 75%, or at least 90%.

Another aspect relates to a modified fungus wherein level of activity of an enzyme causing the formation of gluconolactone or gluconic acid is reduced from about 50% to about 100%, at least 75%, or at least 90%.

Another aspect relates to a modified fungus wherein one or more genes encoding enzymes responsible for the production of one or more products selected from cellobionolactone, cellobionic acid, gluconolactone, and gluconic acid encode an enzyme selected from the group consisting of cellobiose dehydrogenase (CDH), glucooligosaccharide dehydrogenase, glucose dehydrogenase, glucooligosaccharide oxidase, cellobiose oxidase, glucose oxidase, and copper-dependent polysaccharide monooxygenase. A related aspect includes a modified fungus wherein one or more genes encoding a beta-glucosidase is present at higher levels than the unmodified parent fungus, a modified fungus wherein one or more genes encoding a xylanase is present at higher levels than the unmodified parent fungus, or a modified fungus wherein one or more genes encoding a copper-dependent polysaccharide monooxygenase is present at higher levels than the unmodified parent fungus.

One aspect relates to a modified fungus wherein one or more genes encoding enzymes responsible for the production of one or more products selected from cellobionolactone, cellobionic acid, gluconolactone, and gluconic acid encode a cellobiose dehydrogenase (CDH). Related aspects include a modified fungus wherein the amino acid sequence of the cellobiose dehydrogenase (CDH) is selected from a group of polypeptides having at least 90%, 95%, or 99% homology with any of the polypeptides of SEQ ID NOS: 10-12. Related aspects include a modified fungus wherein the cellobiose dehydrogenase (CDH) is CDH1 (SEQ ID NO: 10), CDH2 (SEQ ID NO: 11), or CDH3 (SEQ ID NO: 12). Related aspects include a modified fungus of wherein CDH activity is reduced from about 50% to about 100%, or at least 75%, 90%, or 95%, when measured by a ferricyanide reduction assay.

Related aspects include a modified fungus wherein the level of expression of at least one modified gene encoding a cellobiose dehydrogenase or the level of activity of at least one cellobiose dehydrogenase is reduced or eliminated, and an aspects wherein the level of expression of at least two modified genes encoding cellobiose dehydrogenases or the level of activity of at least two cellobiose dehydrogenases are reduced or eliminated.

Another aspect relates to a modified fungus wherein one or more of the modified genes encode an enzyme selected from the group consisting of glucooligosaccharide dehydrogenase, glucooligosaccharide oxidase, and copper-dependent polysaccharide monooxygenase. Related aspects include a modified fungus wherein one or more of said modified genes encode a glucooligosaccharide oxidase, including a modified fungus wherein the amino acid sequence of the glucooligosaccharide oxidase is selected from a group of polypeptides having at least 90%, 95%, or 99% homology with any of the polypeptides of SEQ ID NOS: 13-14.

Another aspect relates to a modified fungus wherein one or more of the modified genes encode a copper-dependent polysaccharide monooxygenase, including a modified fungus wherein the amino acid sequence of copper-dependent polysaccharide monooxygenase is selected from a group of polypeptides having at least 90%, 95%, or 99% homology with any of the polypeptides of SEQ ID NOS: 15-41.

Another aspect relates to a modified fungus wherein one or more of the modified genes encode an oxidase, including a modified fungus wherein the amino acid sequence of the oxidase is selected from a group of polypeptides having at least 90%, 95, or 99% homology with any of the polypeptides of SEQ ID NOS: 42-52.

Another aspect relates to a modified fungus as described above, further comprising a modified gene encoding a protease wherein the level of expression said modified gene or level of activity of said modified protease is present at lower levels than the unmodified parent fungus.

The present invention is also directed to a composition for the degradation and saccharification of (ligno)cellulosic materials comprising a mixture of enzymes obtained from a modified fungus, wherein said composition has one or more enzymes having cellulase or hemicellulase activities, and lacks or has reduced levels or activities of one or more enzymes responsible for the production of one or more products selected from the group consisting of cellobionolactone, cellobionic acid, gluconolactone, and gluconic acid; wherein production of glucose with said composition in the presence of (ligno)cellulosic materials is enhanced above the endogenous level of glucose produced with a composition which has normal levels or activities of one or more enzymes responsible for the production of one or more products selected from cellobionolactone, cellobionic acid, gluconolactone, and gluconic acid.

A related aspect includes a composition wherein the cellulase is selected from the group consisting of cellobiohydralase, beta-glucosidase, and endoglacanase.

A related aspect includes a composition wherein the hemicellulase is selected from at least one beta-xylosidase, a xylanase, an arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xyloglucanase, and a beta glucosidase.

A related aspect includes a composition wherein the reduced levels or activities of enzymes responsible for the production of one or more products selected from cellobionolactone, cellobionic acid, gluconolactone, gluconic acid is due to the downregulation, deletion, or mutation of at least one enzyme selected from cellobiose dehydrogenase, glucooligosaccharide dehydrogenase, glucose dehydrogenase, glucooligosaccharide oxidase, cellobiose oxidase, glucose oxidase, and copper-dependent polysaccharide monooxygenase.

A related aspect includes a composition wherein at least one of the enzymes of SEQ ID NOS: 10-52 is absent.

A related aspect includes a composition wherein the level or activity of one or more enzymes responsible for the production of one or more products selected from cellobionolactone, cellobionic acid, gluconolactone, gluconic acid is eliminated or reduced in the presence of an inhibiting amount of at least one inhibitor of said enzymes.

A related aspect includes a composition wherein the level or activity of one or more enzymes responsible for the production of one or more products selected from cellobionolactone, cellobionic acid, gluconolactone, gluconic acid is eliminated or reduced by total or partial inactivation of at least one of said enzymes. Another aspect relates to a composition of wherein at wherein the level or activity of one or more enzymes responsible for the production of one or more products selected from cellobionolactone, cellobionic acid, gluconolactone, gluconic acid is eliminated or reduced by total or partial inactivation of at least two of said enzymes.

A related aspect includes a composition wherein the level or activity of one or more enzymes responsible for the production of one or more products selected from cellobionolactone, cellobionic acid, gluconolactone, gluconic acid is eliminated or reduced by removal of at least one of said enzymes.

A related aspect includes a composition wherein at least one of said enzymes is obtained from a modified fungus modified by random mutagenesis.

A related aspect includes a composition wherein at least one of said enzymes is obtained from a modified fungus modified by directed mutagenesis.

The present invention is also directed to a method of increasing saccharification of cellulosic materials comprising: treating the cellulosic material with an enzyme composition comprising enzymes having one or more cellulase or hemicellulase activities; wherein the enzyme composition is obtained from a modified fungus comprising one or more modified genes encoding enzymes responsible for the production of one or more products selected from cellobionolactone, cellobionic acid, gluconolactone, and gluconic acid; wherein the level of expression of said modified genes is eliminated or reduced or the level of activity of modified enzymes encoded by modified genes is reduced or eliminated, compared to the endogenous level of expression or activity in a parent fungus lacking one or more of said modified genes.

A related aspect includes a method of wherein the modified genes encoding enzymes responsible for the production of one or more products selected from cellobionolactone, cellobionic acid, gluconolactone, and gluconic acid are selected from the group consisting of cellobiose dehydrogenases (CDH), glucooligosaccharide dehydrogenases, glucose dehydrogenases, glucooligosaccharide oxidases, cellobiose oxidases, glucose oxidases and copper-dependent polysaccharide monooxygenases.

A related aspect includes a method wherein the level or activity of one or more enzymes responsible for the production of one or more products selected from cellobionolactone, cellobionic acid, gluconolactone, gluconic acid is eliminated or reduced by total or partial inactivation of at least one of said enzymes, and a method wherein at wherein the level or activity of one or more enzymes responsible for the production of one or more products selected from cellobionolactone, cellobionic acid, gluconolactone, gluconic acid is eliminated or reduced by total or partial inactivation of at least two of said enzymes.

A related aspect includes a method wherein the modified fungus is a M. thermophila C1 fungus and derivatives thereof, such as a M. thermophila C1 fungus selected from Garg 27K, (Accession No. VKM F-3500 D) UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190), preferably a M. thermophila C1 fungus derived from UV18-25 (Accession No. VKM F-3631 D). Other aspects include a method wherein the modified fungus comprises at least one cdh gene which is altered, including a cdh1 gene which is disrupted, exemplified by a cdh1 gene was deleted by replacing it with a selection marker, such as a gene encoding the selection marker AmdS. Related aspects include method wherein the modified fungus contains alterations in two cdh genes, such as a cdh1 gene, and a cdh2 gene, which are disrupted by insertion and/or deletion of genetic material, such as a gene encoding a selectable marker.

In some aspects, the present invention comprises a multi-enzyme composition comprising at least one protein degrading a lignocellulosic material or a fragment thereof that has biological activity, wherein the composition has reduced activity for or lacks enzymes which lead to the formation of cellobionolactone/cellobionic acid and/or gluconolactone/gluconic acid.

In some aspects, the multi-enzyme composition comprises at least one cellobiohydrolase, at least one xylanase, at least one endoglucanase, at least one β-glucosidase, at least one β-xylosidase, and at least one accessory enzyme in the absence of enzymes which lead to the formation of cellobionolactone/cellobionic acid and/or gluconolactone/gluconic acid.

In some aspects the reduced enzyme activity leading to the formation of cellobionolactone/cellobionic acid and/or gluconolactone/gluconic acid is about 50% less activity, in other aspects the reduced enzyme activity is 75% or about 90% less activity.

In some aspects the reduced cellobionolactone/cellobionic acid and/or gluconolactone/gluconic acid activity is about 50% less activity, in other aspects the reduced enzyme activity is 75% or about 90% less activity.

In some aspects, between about 50% and about 70% of the enzymes in the multi-enzyme composition are cellobiohydrolases. In some aspects, between about 10% and about 30% of the enzymes in the composition are xylanases. In some aspects, between about 5% and about 15% of the enzymes in the composition are endoglucanases. In some aspects, between about 1% and about 5% of the enzymes in the composition are β-glucosidases. In some aspects, between about 1% and about 3% of the enzymes in the composition are β-xylosidases in the absence of enzymes which lead to the formation of cellobionolactone/cellobionic acid and/or gluconolactone/gluconic acid.

In some aspects, the multi-enzyme composition comprises about 60% cellobiohydrolases, about 20% xylanases, about 10% endoglucanases, about 3% β-glucosidases, about 2% β-xylosidases, and about 5% accessory enzymes in the absence of enzymes which lead to the formation of cellobionolactone/cellobionic acid and/or gluconolactone/gluconic acid.

In some aspects, the xylanases are selected from the group consisting of: endoxylanases, exoxylanases, and β-xylosidases in the absence of enzymes which lead to the formation of cellobionolactone/cellobionic acid and/or gluconolactone/gluconic acid.

In some aspects, the accessory enzymes include an enzyme selected from the group consisting of: cellulase, glucosidase, copper-dependent polysaccharide monooxygenase (or GH61 or polypeptide having cellulolytic enhancing activity), xylanase, xylosidase, ligninase, glucuronidase, arabinofuranosidase, arabinase, arabinogalactanase, ferulic acid esterase, lipase, pectinase, glucomannase, amylase, laminarinase, xyloglucanase, galactanase, galactosidase, glucoamylase, pectate lyase, chitosanase, exo-β-D-glucosaminidase, cellobiose dehydrogenase, and acetylxylan esterase in the absence of enzymes which lead to the formation of cellobionolactone/cellobionic acid and/or gluconolactone/gluconic acid.

In some aspects, the multi-enzyme composition comprises at least one hemicellulase. In some aspects, the hemicellulase is selected from the group consisting of a xylanase, an arabinofuranosidase, an acetyl xylan esterase, a glucuronidase, and endo-galactanase, a mannanase, an endo arabinase, an exo arabinase, an exo-galactanase, a ferulic acid esterase, a galactomannanase, a xylogluconase, and mixtures thereof. In some aspects, the xylanase is selected from the group consisting of endoxylanases, exoxylanase, and β-xylosidase in the absence of enzymes which lead to the formation of cellobionolactone/cellobionic acid and/or gluconolactone/gluconic acid.

In some aspects, the multi-enzyme composition comprises at least one cellulase.

In some aspects, the composition is a crude fermentation product. In some aspects, the composition is a crude fermentation product that has been subjected to a purification step.

In some aspects, the multi-enzyme composition further comprises one or more accessory enzymes. In some aspects, the accessory enzymes include at least one enzyme selected from the group consisting of: cellulase, glucosidase, copper-dependent polysaccharide monooxygenase (or GH61 or polypeptide having cellulolytic enhancing activity), xylanase, xylosidase, ligninase, glucuronidase, arabinofuranosidase, arabinase, arabinogalactanase, ferulic acid esterase, lipase, pectinase, glucomannase, amylase, laminarinase, xyloglucanase, galactanase, galactosidase, glucoamylase, pectate lyase, chitosanase, exo-β-D-glucosaminidase, cellobiose dehydrogenase, and acetylxylan esterase. In some aspects, the accessory enzyme is selected from the group consisting of a glucoamylase, a pectinase, and a ligninase. In some aspects, the accessory enzyme is added as a crude or a semi-purified enzyme mixture. In some aspects, the accessory enzyme is produced by culturing at least one organism on a substrate to produce the enzyme.

In some aspects, the multi-enzyme composition comprises at least one protein for degrading an arabinoxylan-containing material or a fragment thereof that has biological activity.

In some aspects, the composition comprises at least one endoxylanase, at least one β-xylosidase, and at least one arabinofuranosidase. In some aspects, the arabinofuranosidase comprises an arabinofuranosidase with specificity towards single substituted xylose residues, an arabinofuranosidase with specificity towards double substituted xylose residues, or a combination thereof.

In one aspect, the present invention comprises an enzyme mixture which is lacking enzymatic activities causing the formation of cellobionolactone/cellobionic acid and/or gluconolactone/gluconic acid.

In another aspect, the present invention comprises an enzyme mixture which is impaired in enzymatic activities causing the formation of cellobionolactone/cellobionic acid and/or gluconolactone/gluconic acid.

In another aspect the lacking or impaired enzymatic activities are cellobiose dehydrogenases (“CDH”) glucooligosaccharide dehydrogenases, glucose dehydrogenases, glucooligosaccharide oxidases, cellobiose oxidases, glucose oxidases and copper-dependent polysaccharide monooxygenases (or GH61 or polypeptides having cellulolytic enhancing activity).

In another aspect the elimination of enzymatic activities causing the formation of cellobionolactone/cellobionic acid and/or gluconolactone/gluconic acid reduced the acidification of the reaction mixture which improves the activity of the remaining enzymes.

In one aspect the elimination of enzymatic activities is created by disrupting the corresponding gene encoding these activities.

In one aspect the gene encoding CDH1 or encoding CDH2 in Myceliophthora thermophila C1 is knocked out.

In one aspect the genes encoding CDH1 and CDH2 in Myceliophthora thermophila C1 are both knocked out (Double knock out).

In one aspect the enzyme lacking or impaired is at least one CDH selected from a group of polypeptides having at least 60%, more preferably 70%, more preferably 80%, more preferably 90%, more preferably 95%, more preferably 99%, with the CDH1 of SEQ ID No: 10, the CDH2 of SEQ ID No. 11 or the CDH3 of SEQ ID No. 12.

In another aspect a second CDH enzyme, selected from a group of polypeptides having at least 60%, more preferably 70%, more preferably 80%, more preferably 90%, more preferably 95%, more preferably 99%, with the CDH1 of SEQ ID No: 10, the CDH2 of SEQ ID No. 11 or the CDH3 of SEQ ID No. 12, is lacking or impaired.

In one aspect the enzyme lacking or impaired is at least one glucooligosaccharide oxidases (“GOO”) selected from a group of polypeptides having at least 60%, more preferably 70%, more preferably 80%, more preferably 90%, more preferably 95%, more preferably 99%, with the GOOX1 of SEQ ID NO: 13 or the GOOX2 of SEQ ID No. 14.

In another aspect a second GOO enzyme, selected from a group of polypeptides having at least 60%, more preferably 70%, more preferably 80%, more preferably 90%, more preferably 95%, more preferably 99%, with the GOOX1 of SEQ ID No: 13 or the GOOX2 of SEQ ID No. 14, is lacking or impaired.

In one aspect the enzyme lacking or impaired is at least one GH61 family enzyme (“GH61”) selected from a group of polypeptides having at least 60%, more preferably 70%, more preferably 80%, more preferably 90%, more preferably 95%, more preferably 99%, with the GH61 enzymes of SEQ ID NOS: 15-41.

In one aspect the enzyme lacking or impaired is at least one oxidase enzyme selected from a group of polypeptides having at least 60%, more preferably 70%, more preferably 80%, more preferably 90%, more preferably 95%, more preferably 99%, with the oxidase enzymes of SEQ ID NOS: 42-51.

In an aspect the enzyme mixtures produced by knock-out strains produce less gluconic acid in biomass saccharification.

In an aspect the enzyme mixtures produced by knock-out strains show less acidification in biomass saccharification.

In some aspects, the present invention comprises an isolated host cell transfected with a nucleic acid molecule of the present invention. In some aspects, the host cell is a fungus. In some aspects, the host cell is a filamentous fungus. In some aspects, the filamentous fungus is from a genus selected from the group consisting of: Chrysosporium, Thielavia, Talaromyces, Neurospora, Aureobasidium, Filibasidium, Piromyces, Corynascus, Cryptococcus, Acremonium, Tolypocladium, Scytalidium, Schizophyllum, Sporotrichum, Penicillium, Gibberella, Myceliophthora, Mucor, Aspergillus, Fusarium, Humicola, Trichoderma, Talaromyces emersonii and anamorphs and teleomorphs thereof. In some aspects, the host cell is a bacterium.

In some aspects, the genetically modified organism is a plant, alga, fungus or bacterium. In some aspects, the fungus is yeast, mushroom or filamentous fungus. In some aspects, the filamentous fungus is from a genus selected from the group consisting of: Chrysosporium, Thielavia, Neurospora, Aureobasidium, Filibasidium, Piromyces, Corynascus, Cryptococcus, Acremonium, Tolypocladium, Scytalidium, Schizophyllum, Sporotrichum, Penicillium, Talaromyces, Gibberella, Myceliophthora, Mucor, Aspergillus, Fusarium, Humicola, and Trichoderma. In some aspects, the filamentous fungus is selected from the group consisting of: Trichoderma reesei, Trichoderma harzanium, Myceliophthora thermophila, Aspergillus niger, Aspergillus oryzae, Aspergillus japonicus, Aspergillus niger Penicillium canescens, Penicillium solitum, Penicillium funiculosum, Talaromyces emersonii, Talaromyces flavus, and Myceliophthora thermophila.

In some aspects, the genetically modified organism is a plant, alga, fungus or bacterium. In some aspects, the fungus is yeast, mushroom or filamentous fungus. In some aspects, the filamentous fungus is from a genus selected from the group consisting of: Chrysosporium, Thielavia, Neurospora, Aureobasidium, Filibasidium, Piromyces, Corynascus, Cryptococcus, Acremonium, Tolypocladium, Scytalidium, Schizophyllum, Sporotrichum, Penicillium, Talaromyces, Gibberella, Myceliophthora, Mucor, Aspergillus, Fusarium, Humicola, and Trichoderma. In some aspects, the filamentous fungus is selected from the group consisting of: Trichoderma reesei, Trichoderma harzanium, Myceliophthora thermophila, Aspergillus niger, Aspergillus oryzae, Aspergillus japonicus, Aspergillus niger Penicillium canescens, Penicillium solitum, Penicillium funiculosum, Talaromyces emersonii, Talaromyces flavus, and Myceliophthora thermophila and other mesophilic, and thermophilic fungal strains.

In an aspect in biomass saccharification the enzyme mixtures produced by knock-out strains released more glucose than the decrease in gluconic acid.

As used herein the terms “lignin” or “lignen” refers to complex polymers, the chief noncarbohydrate constituent, that binds to cellulose fibers and hardens and strengthens the cell walls of plants. Lignin is an integral part of the secondary cell walls of many plants and some algae. Lignin acts to hold together cellulose and hemicelluose, which are important ingredients in making ethanol.

As used herein the terms “biomass” or “lignocellulosic material” includes materials containing cellulose and/or hemicellulose. Generally, these materials also contain pectin, lignin, protein, carbohydrates (such as starch and sugar) and ash. Lignocellulose is generally found, for example, in the stems, leaves, hulls, husks, and cobs of plants or leaves, branches, and wood of trees.

The process of converting less or more complex carbohydrates (such as starch, cellulose or hemicellulose) into fermentable sugars is also referred to herein as “saccharification.”

Fermentable sugars, as used herein, refers to simple sugars, such as glucose, xylose, arabinose, galactose, mannose, rhamnose, sucrose and fructose.

Biomass can include virgin biomass and/or non-virgin biomass such as agricultural biomass, commercial organics, construction and demolition debris, municipal solid waste, waste paper and yard waste. Common forms of biomass include trees, shrubs and grasses, wheat, wheat straw, sugar cane bagasse, sugar beet, soybean, corn, corn husks, corn kernel including fiber from kernels, products and by-products from milling of grains such as corn, tobacco, wheat and barley (including wet milling and dry milling) as well as municipal solid waste, waste paper and yard waste. The biomass can also be, but is not limited to, herbaceous material, agricultural residues, forestry residues, municipal solid wastes, waste paper, and pulp and paper mill residues. “Agricultural biomass” includes branches, bushes, canes, corn and corn husks, energy crops, algae, fruits, flowers, grains, grasses, herbaceous crops, leaves, bark, needles, logs, roots, saplings, short rotation woody crops, shrubs, switch grasses, trees, vegetables, fruit peels, vines, sugar beet pulp, wheat midlings, oat hulls, peat moss, mushroom compost and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination or mixture thereof.

Energy crops are fast-growing crops that are grown for the specific purpose of producing energy, including without limitation, biofuels, from all or part of the plant. Energy crops can include crops that are grown (or are designed to grow) for their increased cellulose, xylose and sugar contents. Examples of such plants include, without limitation, switchgrass, willow and poplar. Energy crops may also include algae, for example, designer algae that are genetically engineered for enhanced production of hydrogen, alcohols, and oils, which can be further processed into diesel and jet fuels, as well as other bio-based products.

Biomass high in starch, sugar, or protein such as corn, grains, fruits and vegetables are usually consumed as food. Conversely, biomass high in cellulose, hemicellulose and lignin are not readily digestible and are primarily utilized for wood and paper products, animal feed, fuel, or are typically disposed. Generally, the substrate is of high lignocellulose content, including distillers' dried grains corn stover, corn cobs, rice straw, wheat straw, hay, sugarcane bagasse, sugar cane pulp, citrus peels and other agricultural biomass, switchgrass, forestry wastes, poplar wood chips, pine wood chips, sawdust, yard waste, and the like, including any combination thereof.

Due in part to the many components that comprise biomass and lignocellulosic materials, enzymes or a mixture of enzymes capable of degrading xylan, lignin, protein, and carbohydrates are needed to achieve saccharification. The present invention includes enzymes or compositions thereof with, for example, oxidoreductases, cellobiohydrolase, endoglucanase, xylanase, β-glucosidase, and hemicellulase activities.

Fermentable sugars can be converted to useful value-added fermentation products, non-limiting examples of which include amino acids, vitamins, pharmaceuticals, animal feed supplements, specialty chemicals, chemical feedstocks, plastics, solvents, fuels, or other organic polymers, lactic acid, and ethanol, including fuel ethanol. Specific value-added products that may be produced by the methods of the invention include, but not limited to, biofuels (including ethanol and butanol); lactic acid; plastics; specialty chemicals; organic acids, including citric acid, succinic acid and maleic acid; solvents; animal feed supplements; pharmaceuticals; vitamins; amino acids, such as lysine, methionine, tryptophan, threonine, and aspartic acid; industrial enzymes, such as proteases, cellulases, amylases, glucanases, xylanases, arabinanases, lactases, lipases, esterases, lyases, oxidoreductases, transferases; and chemical feedstocks.

In one aspect, the present invention includes proteins isolated from, or derived from the knowledge of enzymes from, a fungus such as Myceliopthora (previously known as C. lucknowense) or a mutant or other derivative thereof, and more particularly, from the fungal strain denoted herein as C1 (Accession No. VKM F-3500 D). Preferably, the proteins of the invention possess enzymatic activity. As described in U.S. Pat. No. 6,015,707 or U.S. Pat. No. 6,573,086 a strain called C1 (Accession No. VKM F-3500 D), was isolated from samples of forest alkaline soil from Sola Lake, Far East of the Russian Federation. This strain was deposited at the All-Russian Collection of Microorganisms of Russian Academy of Sciences (VKM), Bakhurhina St. 8, Moscow, Russia, 113184, under the terms of the Budapest Treaty on the International Regulation of the Deposit of Microorganisms for the Purposes of Patent Procedure on Aug. 29, 1996, as Chrysosporium lucknowense Garg 27K, VKM F-3500 D. Various mutant strains of C1 have been produced and these strains have also been deposited at the All-Russian Collection of Microorganisms of Russian Academy of Sciences (VKM), Bakhurhina St. 8, Moscow, Russia, 113184, under the terms of the Budapest Treaty on the International Regulation of the Deposit of Microorganisms for the Purposes of Patent Procedure on Sep. 2, 1998 or at the Centraal Bureau voor Schimmelcultures (CBS), Uppsalalaan 8, 3584 CT Utrecht, The Netherlands for the purposes of Patent Procedure on Dec. 5, 2007. For example, Strain C1 was mutagenised by subjecting it to ultraviolet light to generate strain UV13-6 (Accession No. VKM F-3632 D). This strain was subsequently further mutated with N-methyl-N′-nitro-N-nitrosoguanidine to generate strain NG7C-19 (Accession No. VKM F-3633 D). This latter strain in turn was subjected to mutation by ultraviolet light, resulting in strain UV18-25 (Accession No. VKM F-3631 D). This strain in turn was again subjected to mutation by ultraviolet light, resulting in strain W1L (Accession No. CBS122189), which was subsequently subjected to mutation by ultraviolet light, resulting in strain W1L#100L (Accession No. CBS122190). Strain C1 was initially classified as a Chrysosporium lucknowense based on morphological and growth characteristics of the microorganism, as discussed in detail in U.S. Pat. No. 6,015,707, U.S. Pat. No. 6,573,086 and patent PCT/NL2010/000045. The C1 strain was subsequently reclassified as Myceliophthora. thermophila based on genetic tests. C. luknowense has also appeared in the literature as Sporotrichum thermophile.

While the examples below are shown in strains of C1, the concepts demonstrated herein can be applied to other microorganisms such as, but not limited to, hyphal fungi which express enzymes promoting the formation of cellobionolactone/cellobionic acid and/or gluconolactone/gluconic acid.

As used herein, “oxidoreductase” refers to an enzyme that catalyzes the transfer of electrons from one molecule (the reductant, also called the hydride or electron donor) to another (the oxidant, also called the idem or electron acceptor). A few of the oxidoreductase enzymes are listed below. This list is not exhaustive. Other oxidoreductase and their activity are well known to those skilled in the art.

“Oxidase” refers to any enzyme that catalyzes an oxidation-reduction reaction involving molecular oxygen (O₂) as the electron acceptor. Oxidases include but are not limited to glucose oxidase, cellobiose oxidase and oligosaccharide oxidase.

“Monooxygenases” are oxidoreductases that induce the incorporation of one atom of oxygen from O₂ into the substance being oxidized.

“Hydroxylases” are oxidoreductases that induce the introduction of a hydroxyl group in the substance being oxidized.

“Dehydrogenases” refer to enzymes that catalyze the removal of hydrogen from organic compounds. Dehydrogenases include, but are not limited to glucooligosaccharide dehydrogenases, glucose dehydrogenases, or cellobiose dehydrogenases.

“Cellobiose dehydrogenase” refers to a protein that oxidizes cellobiose to cellobionolactone and/or glucose to gluconolactone.

Other examples of oxidases are those catalyzing the oxidation of sugars such as glucose oxidase, galactose oxidase and hexose oxidase.

As used herein, “carbohydrase” refers to any protein that catalyzes the hydrolysis of carbohydrates. “Glycoside hydrolase”, “glycosyl hydrolase” or “glycosidase” refers to a protein that catalyzes the hydrolysis of the glycosidic bonds between carbohydrates or between a carbohydrate and a non-carbohydrate residue. Endoglucanases, cellobiohydrolases, β-glucosidases, α-glucosidases, xylanases, β-xylosidases, alpha-xylosidases, galactanases, α-galactosidases, β-galactosidases, α-amylases, glucoamylases, endo-arabinases, arabinofuranosidases, mannanases, β-mannosidases, pectinases, acetyl xylan esterases, acetyl mannan esterases, ferulic acid esterases, coumaric acid esterases, pectin methyl esterases, and chitosanases are examples of glycosidases.

“Cellulose” is a linear beta-(1-4) glucan consisting of anhydrocellobiose units. Cellulases include endoglucanases, cellobiohydrolases, and β-glucosidases. “Cellulase” refers to a protein that catalyzes the hydrolysis of 1,4-β-D-glycosidic linkages in cellulose; cellulose derivatives (such as carboxymethylcellulose and hydroxyethylcellulose); plant lignocellulosic materials, beta-D-glucans or xyloglucans.

“Endoglucanase” refers to a protein that catalyzes the hydrolysis of cellulose to oligosaccharide chains at random locations by means of an endoglucanase activity.

“Cellobiohydrolase” refers to a protein that catalyzes the hydrolysis of cellulose to cellobiose via an exoglucanase activity, sequentially releasing molecules of cellobiose from the reducing or non-reducing ends of cellulose or cello-oligosaccharides.

“β-glucosidase” refers to an enzyme that catalyzes the conversion of cellobiose and oligosaccharides to glucose.

“Hemicellulase” refers to a protein that catalyzes the hydrolysis of hemicellulose, such as that found in lignocellulosic materials. Hemicelluloses are complex polymers, and their composition often varies widely from organism to organism, and from one tissue type to another. Hemicelluloses include a variety of compounds, such as xylans, arabinoxylans, xyloglucans, mannans, glucomannans, pectins, polygalacturonan, rhamnogalacturonan, xylogalacturonan and galacto(gluco)mannans. Hemicellulose can also contain glucan, which is a general term for beta-linked glucose residues. In general, a main component of hemicellulose is beta-1,4-linked xylose, a five carbon sugar. However, this xylose is often branched as beta-1,3 linkages or beta-1,2 linkages, and can be substituted with linkages to arabinose, galactose, mannose, glucuronic acid, or by esterification to acetic acid. The composition, nature of substitution, and degree of branching of hemicellulose is very different in dicotyledonous plants (dicots, i.e., plant whose seeds have two cotyledons or seed leaves such as lima beans, peanuts, almonds, peas, kidney beans) as compared to monocotyledonous plants (monocots; i.e., plants having a single cotyledon or seed leaf such as corn, wheat, rice, grasses, barley). In dicots, hemicellulose is comprised mainly of xyloglucans that are 1,4-beta-linked glucose chains with 1,6-alpha-linked xylosyl side chains. In monocots, including most grain crops, the principal components of hemicellulose are heteroxylans. These are primarily comprised of 1,4-beta-linked xylose backbone polymers with 1,2- or 1,3-alpha linkages to arabinose, linkage of galactose and mannose to arabinose or xylose in side chains, as well as xylose modified by ester-linked acetic acids. Also present are branched beta glucans comprised of 1,3- and 1,4-beta-linked glucosyl chains. In monocots, cellulose, heteroxylans and beta glucans are present in roughly equal amounts, each comprising about 15-25% of the dry matter of cell walls. Hemicellulolytic enzymes, i.e. Hemicellulases, include both endo-acting and exo-acting enzymes, such as xylanases, β-xylosidases. alpha-xylosidases, galactanases, α-galactosidases, β-galactosidases, endo-arabinases, arabinofuranosidases, mannanases, β-mannosidases. Hemicellulases also include the accessory enzymes, such as acetylesterases, ferulic acid esterases, and coumaric acid esterases. Among these, xylanases and acetyl xylan esterases cleave the xylan and acetyl side chains of xylan and the remaining xylo-oligomers are unsubstituted and can thus be hydrolyzed with β-xylosidase only. In addition, several less known side activities have been found in enzyme preparations which hydrolyze hemicellulose. Accordingly, xylanases, acetylesterases and β-xylosidases are examples of hemicellulases.

“Xylanase” specifically refers to an enzyme that hydrolyzes the β-1,4 bond in the xylan backbone, producing short xylooligosaccharides.

“β-Mannanase” or “endo-1,4-β-mannosidase” refers to a protein that hydrolyzes mannan-based hemicelluloses (mannan, glucomannan, galacto(gluco)mannan) and produces short β-1,4-mannooligosaccharides.

“Mannan endo-1,6-α-mannosidase” refers to a protein that hydrolyzes 1,6-α-mannosidic linkages in unbranched 1,6-mannans.

“β-Mannosidase” (β-1,4-mannoside mannohydrolase; EC 3.2.1.25) refers to a protein that catalyzes the removal of β-D-mannose residues from the nonreducing ends of oligosaccharides.

“Galactanase”, “endo-β-1,6-galactanse” or “arabinogalactan endo-1,4-β-galactosidase” refers to a protein that catalyzes the hydrolysis of endo-1,4-β-D-galactosidic linkages in arabinogalactans.

“Glucoamylase” refers to a protein that catalyzes the hydrolysis of terminal 1,4-linked α-D-glucose residues successively from non-reducing ends of the glycosyl chains in starch with the release of β-D-glucose.

“β-hexosaminidase” or “β-N-acetylglucosaminidase” refers to a protein that catalyzes the hydrolysis of terminal N-acetyl-D-hexosamine residues in N-acetyl-β-D-hexosamines.

“α-L-arabinofuranosidase”, “α-N-arabinofuranosidase”, “α-arabinofuranosidase”, “arabinosidase” or “arabinofuranosidase” refers to a protein that hydrolyzes arabinofuranosyl-containing hemicelluloses. Some of these enzymes remove arabinofuranoside residues from O-2 or O-3 single substituted xylose residues, as well as from O-2 and/or O-3 double substituted xylose residues.

“Endo-arabinase” refers to a protein that catalyzes the hydrolysis of 1,5-α-arabinofuranosidic linkages in 1,5-arabinans.

“Exo-arabinase” refers to a protein that catalyzes the hydrolysis of 1,5-α-linkages in 1,5-arabinans or 1,5-α-L arabino-oligosaccharides, releasing mainly arabinobiose, although a small amount of arabinotriose can also be liberated.

“β-xylosidase” refers to a protein that hydrolyzes short 1,4-β-D-xylooligomers into xylose.

“Chitosanase” refers to a protein that catalyzes the endohydrolysis of β-1,4-linkages between D-glucosamine residues in acetylated chitosan (i.e., deacetylated chitin).

“Exo-polygalacturonase” refers to a protein that catalyzes the hydrolysis of terminal alpha 1,4-linked galacturonic acid residues from non-reducing ends thus converting polygalacturonides to galacturonic acid.

“Acetyl xylan esterase” refers to a protein that catalyzes the removal of the acetyl groups from xylose residues. “Acetyl mannan esterase” refers to a protein that catalyzes the removal of the acetyl groups from mannose residues. “ferulic esterase” or “ferulic acid esterase” refers to a protein that hydrolyzes the ester bond between the arabinose substituent group and ferulic acid. “Coumaric acid esterase” refers to a protein that hydrolyzes the ester bond between the arabinose substituent group and coumaric acid. Acetyl xylan esterases, ferulic acid esterases and pectin methyl esterases are examples of carbohydrate esterases.

“Pectate lyase” and “pectin lyases” refer to proteins that catalyze the cleavage of 1,4-α-D-galacturonan by beta-elimination acting on polymeric and/or oligosaccharide substrates (pectates and pectins, respectively).

“Endo-1,3-β-glucanase” or “laminarinase” refers to a protein that catalyzes the cleavage of 1,3-linkages in β-D-glucans such as laminarin or lichenin. Laminarin is a linear polysaccharide made up of β-1,3-glucan with β-1,6-linkages.

“Lichenase” refers to a protein that catalyzes the hydrolysis of lichenan, a linear, 1,3-1,4-β-D glucan.

Rhamnogalacturonan is composed of alternating α-1,4-rhamnose and α-1,2-linked galacturonic acid, with side chains linked 1,4 to rhamnose. The side chains include Type I galactan, which is β-1,4-linked galactose with α-1,3-linked arabinose substituents; Type II galactan, which is β-1,3-1,6-linked galactoses (very branched) with arabinose substituents; and arabinan, which is α-1,5-linked arabinose with α-1,3-linked arabinose branches. The galacturonic acid substituents may be acetylated and/or methylated.

“Rhamnogalacturonan acetylesterase” refers to a protein that catalyzes the removal of the acetyl ester-linked to the highly branched rhamnogalacturonan (hairy) regions of pectin.

“Rhamnogalacturonan lyase” refers to a protein that catalyzes the degradation of the rhamnogalacturonan backbone of pectin via a β-elimination mechanism (see, e.g., Pages et al., J. Bacteriol. 185:4727-4733 (2003)).

“Alpha-rhamnosidase” refers to a protein that catalyzes the hydrolysis of terminal non-reducing α-L-rhamnose residues in α-L-rhamnosides.

Glycosidases (glycoside hydrolases; GH), a large family of enzymes that includes cellulases and hemicellulases, catalyze the hydrolysis of glycosidic linkages, predominantly in carbohydrates. Glycosidases such as the proteins of the present invention may be assigned to families on the basis of sequence similarities, and there are now over 100 different such families defined (see the CAZy (Carbohydrate Active EnZymes database) website, maintained by the Architecture of Fonction de Macromolecules Biologiques of the Centre National de la Recherche Scientifique, which describes the families of structurally-related catalytic and carbohydrate-binding modules (or functional domains) of enzymes that degrade, modify, or create glycosidic bonds; Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12). Because there is a direct relationship between the amino acid sequence of a protein and its folding similarities, such a classification reflects the structural features of these enzymes and their substrate specificity. Such a classification system can help to reveal the evolutionary relationships between these enzymes and provide a convenient tool to determine information such as an enzyme's activity and function. Thus, enzymes assigned to a particular family based on sequence homology with other members of the family are expected to have similar enzymatic activities and related substrate specificities. CAZy family classifications also exist for glycosyltransferases (GT), polysaccharide lyases (PL), and carbohydrate esterases (CE). Likewise, sequence homology may be used to identify particular domains within proteins, such as carbohydrate binding modules (CBMs; also known as carbohydrate binding domains (CBDs), sometimes called cellulose binding domains). The CAZy homologies of proteins of the present invention are disclosed below. An enzyme assigned to a particular CAZy family may exhibit one or more of the enzymatic activities or substrate specificities associated with the CAZy family. In other aspects, the enzymes of the present invention may exhibit one or more of the enzyme activities discussed above.

Certain proteins used in the multi-enzyme compositions of the present invention may be classified as “Family 61 glycosidases” based on homology of the polypeptides to CAZy Family GH61. Family 61 glycosidases may exhibit oxidative activity towards biopolymers including, but not limited to, cellulose, hemicellulose, chitin, chitosan, amylose, amylopectin, pectin and lignin. The oxidative activity towards the biopolymers may results in an enhancing effect on the degradation of the corresponding biopolymer. “Family 61 glycosidases” polypeptides are provided as SEQ ID NO:15-41 “Cellulolytic enhancing activity” refers to a biological activity that enhances the hydrolysis of a cellulosic material by proteins having cellulolytic activity. This enhancing activity is expected to be related by the cellulose oxidizing activity of the GH61 enzyme.

“Hemicellulolytic enhancing activity” refers to a biological activity that enhances the hydrolysis of a hemicellulosic material by proteins having hemicellulolytic activity. This enhancing activity is expected to be related by the hemicellulose oxidizing activity of the GH61 enzyme. As used herein, “hemicellulostic materials” include, but are not limited to, xylan, glucuronoxylan, arabinoxylan, glucomannan, and xyloglucan.

“Chitinolytic enhancing activity” refers to a biological activity that enhances the hydrolysis of a chitinoic material by proteins having chitinase activity. This enhancing activity is expected to be related by the chitin oxidizing activity of the GH61 enzyme.

“Amylolytic enhancing activity” refers to a biological activity that enhances the hydrolysis of a amylosic material by proteins having amylase activity. This enhancing activity is expected to be related by the amylose oxidizing activity of the GH61 enzyme.

“Amylopectinolytic enhancing activity” refers to a biological activity that enhances the hydrolysis of a amylopectinoic materials by proteins having amylopectinase activity. This enhancing activity is expected to be related by the amylopectin oxidizing activity of the GH61 enzyme.

“Pectinolytic enhancing activity” refers to a biological activity that enhances the hydrolysis of a pectinoic materials by proteins having pectinase activity. This enhancing activity is expected to be related by the pectin oxidizing activity of the GH61 enzyme.

“Ligninolytic enhancing activity” refers to a biological activity that enhances the hydrolysis of a lignolytic materials by proteins having ligninase activity. This enhancing activity is expected to be related by the lignin oxidizing activity of the GH61 enzyme.

“Cellobiose dehydrogenases” and “cellobiose oxidases” are oxidoreductases that oxidize cellobiose to cellobiono-1,5-lactone and or glucose to gluconolactone and can utilize electron acceptors including, but not limited to, molecular oxygen, CDH-like reductases, GH61 enzymes, cytochrome c and FeIII.

Proteins used in the multi-enzyme compositions of the present invention may also include homologues, variants, and fragments of the proteins disclosed herein. The protein fragments include, but are not limited to, fragments comprising a catalytic domain (CD) and/or a carbohydrate binding module (CBM) (also known as a cellulose-binding domain; both can be referred to herein as CBM). The identity and location of domains within proteins of the present invention are disclosed in detail below. The present invention encompasses all combinations of the disclosed domains. For example, a protein fragment may comprise a CD of a protein but not a CBM of the protein or a CBM of a protein but not a CD. Similarly, domains from different proteins may be combined. Protein fragments comprising a CD, CBM or combinations thereof for each protein disclosed herein can be readily produced using standard techniques known in the art. In some aspects, a protein fragment comprises a domain of a protein that has at least one biological activity of the full-length protein. Homologues or variants of proteins of the invention that have at least one biological activity of the full-length protein are described in detail below. As used herein, the phrase “biological activity” of a protein refers to any function(s) exhibited or performed by the protein that is ascribed to the naturally occurring form of the protein as measured or observed in vitro or in vivo. In certain aspects, a protein fragment comprises a domain of a protein that has the catalytic activity of the full-length enzyme.

As used herein, reference to an isolated protein or polypeptide in the present invention, including any of the enzymes disclosed herein, includes full-length proteins and their glycosylated or otherwise modified forms, fusion proteins, or any fragment or homologue or variant of such a protein. More specifically, an isolated protein, such as an enzyme according to the present invention, is a protein (including a polypeptide or peptide) that has been removed from its natural milieu (i.e., that has been subject to human manipulation) and can include purified proteins, partially purified proteins, recombinantly produced proteins, synthetically produced proteins, proteins complexed with lipids, soluble proteins, and isolated proteins associated with other proteins, for example. As such, “isolated” does not reflect the extent to which the protein has been purified. Preferably, an isolated protein of the present invention is produced recombinantly. In addition, and by way of example, a “M. thermophila protein” or “M. thermophila enzyme” refers to a protein (generally including a homologue or variant of a naturally occurring protein) from Myceliophthora thermophila or to a protein that has been otherwise produced from the knowledge of the structure (e.g., sequence) and perhaps the function of a naturally occurring protein from Myceliophthora thermophila. In other words, a M. thermophila protein includes any protein that has substantially similar structure and function of a naturally occurring M. thermophila protein or that is a biologically active (i.e., has biological activity) homologue or variant of a naturally occurring protein from M. thermophila as described in detail herein. As such, a M. thermophila protein can include purified, partially purified, recombinant, mutated/modified and synthetic proteins.

According to the present invention, the terms “modification,” “mutation,” and “variant” can be used interchangeably, particularly with regard to the modifications/mutations to the amino acid sequence of a M. thermophila protein (or nucleic acid sequences) described herein. An isolated protein according to the present invention can be isolated from its natural source, produced recombinantly or produced synthetically.

According to the present invention, the terms “modification” and “mutation” can be used interchangeably, particularly with regard to the modifications/mutations to the primary amino acid sequences of a protein or peptide (or nucleic acid sequences) described herein. The term “modification” can also be used to describe post-translational modifications to a protein or peptide including, but not limited to, methylation, farnesylation, carboxymethylation, geranyl geranylation, glycosylation, phosphorylation, acetylation, myristoylation, prenylation, palmitation, and/or amidation. Modification can also include the cleavage of a signal peptide, or methionine, or other portions of the peptide that require cleavage to generate the mature peptide.

As used herein, the terms “homologue” or “variants” are used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide), insertions and/or substitutions; changes in stereochemistry of one or a few atoms; and/or minor derivatizations, including but not limited to for example: methylation, glycosylation and phosphorylation. A homologue or variant can have either enhanced, decreased, or substantially similar properties as compared to the naturally occurring protein or peptide. A homologue or variant can include an agonist of a protein or an antagonist of a protein.

Homologues or variants can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Homologous can also be the result of a gene duplication and rearrangement, resulting in a different location. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues or variants can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications of a protein, such as in a homologue or variant, may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

Modified genes include natural genes modified by substitution, insertion, and/or deletion of single or multiple nucleotide sequences, which can occur within the coding sequence including exons of regions encoding a polypeptide, or in flanking regions, such as regulatory regions typically upstream (e.g., promoters, enhancers, and related sequences), downstream (e.g., transcriptional termination, and poly(A) signals), or internal regions (e.g., introns) that affect the transcription, translation, and/or activation of a polypeptide or regulatory molecule of interest. Activation of a polypeptide, for example, may require removal of one or more N-terminal, C-terminal, or internal polypeptide regions, and/or post-translational modification of specific amino acid residues, such as by glycosylation, amidation, etc., that may alter the targeting, degradation, catalytic activity, of an enzyme.

According to the present invention, an isolated protein, including a biologically active homologue, variant, or fragment thereof, has at least one characteristic of biological activity of a wild-type, or naturally occurring, protein. As discussed above, in general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). The biological activity of a protein of the present invention can include an enzyme activity (catalytic activity and/or substrate binding activity), such as oxidases, oxygenases, monoxygenases, Baeyer-Villiger monooxygenases, dioxygenases, peroxidases, dehydrogenases, reductases that catalyze an oxidation-reduction reaction or any other activity disclosed herein. Specific biological activities of the proteins disclosed herein are described in detail above and in the Examples. Methods of detecting and measuring the biological activity of a protein of the invention include, but are not limited to, the assays described in the Examples section below. Such assays include, but are not limited to, measurement of enzyme activity (e.g., catalytic activity), measurement of substrate binding, and the like. It is noted that an isolated protein of the present invention (including homologues or variants) is not required to have a biological activity such as catalytic activity. A protein can be a truncated, mutated or inactive protein, or lack at least one activity of the wild-type enzyme, for example. Inactive proteins may be useful in some screening assays, for example, or for other purposes such as antibody production.

Methods to measure protein expression levels of a protein according to the invention include, but are not limited to: western blotting, immunocytochemistry, flow cytometry or other immunologic-based assays; assays based on a property of the protein including but not limited to, ligand binding or interaction with other protein partners.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402); (2) a BLAST 2 alignment (using the parameters described below); (3) PSI-BLAST with the standard default parameters (Position-Specific Iterated BLAST; and/or (4) CAZy homology determined using standard default parameters from the Carbohydrate Active EnZymes database (Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12) and/or applying a similar strategy using databases such as the Foly database (website: foly.esil.univ-mrs.fr) and the PeroxiBase (website: peroxibase.isb-sib.ch).

It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues or variants. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.
For blastn, using 0 BLOSUM62 matrix:
Reward for match=1
Penalty for mismatch=−2
Open gap (5) and extension gap (2) penalties
gap x_dropoff (50) expect (10) word size (11) filter (on)
For blastp, using 0 BLOSUM62 matrix:
Open gap (11) and extension gap (1) penalties
gap x_dropoff (50) expect (10) word size (3) filter (on).

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another aspect, a protein of the present invention, including a homologue or variant, includes a protein having an amino acid sequence that is sufficiently similar to a natural amino acid sequence that a nucleic acid sequence encoding the homologue or variant is capable of hybridizing under moderate, high or very high stringency conditions (described below) to (i.e., with) a nucleic acid molecule encoding the natural protein (i.e., to the complement of the nucleic acid strand encoding the natural amino acid sequence). Preferably, a homologue or variant of a protein of the present invention is encoded by a nucleic acid molecule comprising a nucleic acid sequence that hybridizes under low, moderate, or high stringency conditions to the complement of a nucleic acid sequence that encodes a protein comprising, consisting essentially of, or consisting of, an amino acid sequence represented by any of SEQ ID NO: Such hybridization conditions are described in detail below.

A nucleic acid sequence complement of nucleic acid sequence encoding a protein of the present invention refers to the nucleic acid sequence of the nucleic acid strand that is complementary to the strand which encodes the protein. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence such as the amino acid sequences of SEQ ID NO:. Methods to deduce a complementary sequence are known to those skilled in the art. It should be noted that since nucleic acid sequencing technologies are not entirely error-free, the sequences presented herein, at best, represent apparent sequences of the proteins of the present invention.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular aspects, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular aspects, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

The minimum size of a protein and/or homologue or variant of the present invention is a size sufficient to have biological activity or, when the protein is not required to have such activity, sufficient to be useful for another purpose associated with a protein of the present invention, such as for the production of antibodies that bind to a naturally occurring protein. In one aspect, the protein of the present invention is at least 20 amino acids in length, or at least about 25 amino acids in length, or at least about 30 amino acids in length, or at least about 40 amino acids in length, or at least about 50 amino acids in length, or at least about 60 amino acids in length, or at least about 70 amino acids in length, or at least about 80 amino acids in length, or at least about 90 amino acids in length, or at least about 100 amino acids in length, or at least about 125 amino acids in length, or at least about 150 amino acids in length, or at least about 175 amino acids in length, or at least about 200 amino acids in length, or at least about 250 amino acids in length, and so on up to a full length of each protein, and including any size in between in increments of one whole integer (one amino acid). There is no limit, other than a practical limit, on the maximum size of such a protein in that the protein can include a portion of a protein or a full-length protein, plus additional sequence (e.g., a fusion protein sequence), if desired.

The present invention also includes a fusion protein that includes a domain of a protein of the present invention (including a homologue or variant) attached to one or more fusion segments, which are typically heterologous in sequence to the protein sequence (i.e., different than protein sequence). Suitable fusion segments for use with the present invention include, but are not limited to, segments that can: enhance a protein's stability; provide other desirable biological activity; and/or assist with the purification of the protein (e.g., by affinity chromatography). A suitable fusion segment can be a domain of any size that has the desired function (e.g., imparts increased stability, solubility, action or biological activity; and/or simplifies purification of a protein). Fusion segments can be joined to amino and/or carboxyl termini of the domain of a protein of the present invention and can be susceptible to cleavage in order to enable straight-forward recovery of the protein. Fusion proteins are preferably produced by culturing a recombinant cell transfected with a fusion nucleic acid molecule that encodes a protein including the fusion segment attached to either the carboxyl and/or amino terminal end of a domain of a protein of the present invention. Accordingly, proteins of the present invention also include expression products of gene fusions (for example, used to overexpress soluble, active forms of the recombinant protein), of mutagenized genes (such as genes having codon modifications to enhance gene transcription and translation), and of truncated genes (such as genes having membrane binding modules removed to generate soluble forms of a membrane protein, or genes having signal sequences removed which are poorly tolerated in a particular recombinant host).

In one aspect of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived.

The present invention also provides enzyme combinations that break down or modify lignin material, reducing or preventing unwanted adsorption of other components of multi-enzyme compositions applied Such enzyme combinations or mixtures can include a multi-enzyme composition that contains at least one protein of the host organism or one or more enzymes or other proteins from other microorganisms, plants, or similar organisms. Synergistic enzyme combinations and related methods are contemplated. The invention includes methods to identify the optimum ratios and compositions of enzymes with which to degrade each lignin and lignocellulosic material. These methods entail tests to identify the optimum enzyme composition and ratios for efficient conversion of any biomass substrate to its constituent sugars. The Examples below include assays that may be used to identify optimum ratios and compositions of enzymes with which to degrade lignocellulosic materials.

Any combination of the proteins disclosed herein is suitable for use in the multi-enzyme compositions of the present invention. It is to be understood that any of the enzymes described specifically herein can be combined with any one or more of the enzymes described herein or with any other available and suitable enzymes, to produce a multi-enzyme composition. The invention is not restricted or limited to the specific exemplary combinations listed below.

One or more components of a multi-enzyme composition (other than proteins of the present invention) can be obtained from or derived from a microbial, plant, or other source or combination thereof, and will contain enzymes capable of performing oxidation-reduction reactions. Examples of enzymes included in the multi-enzyme compositions of the invention include oxidases, oxygenases, monoxygenases, Baeyer-Villiger monooxygenases, dioxygenases, peroxidases, dehydrogenases, reductases that catalyze an oxidation-reduction reaction.

The multi-enzyme compositions of the invention can also include cellulases, hemicellulases (such as xylanases, including endoxylanases, exoxylanases, and β-xylosidases; mannanases, including endomannanases, exomannanases, and β-mannosidases), ligninases, amylases, glucuronidases, proteases, esterases (including ferulic acid esterase), lipases, glucosidases (such as β-glucosidase), and xyloglucanases.

While the multi-enzyme composition may contain many types of enzymes, mixtures comprising enzymes that increase or enhance sugar release from biomass are contemplated, which may include hemicellulases. In one aspect, the hemicellulase is selected from a xylanase, an arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xyloglucanase, or mixtures of any of these. In particular, the enzymes can include glucoamylase, β-xylosidase and/or β-glucosidase. Also preferred are mixtures comprising enzymes that are capable of degrading cell walls and releasing cellular contents.

The enzymes of the multi-enzyme composition can be provided by a variety of sources. In one aspect, the enzymes can be produced by growing organisms such as bacteria, algae, fungi, and plants which produce the enzymes naturally or by virtue of being genetically modified to express the enzyme or enzymes. In another aspect, at least one enzyme of the multi-enzyme composition is a commercially available enzyme.

In some aspects, the multi-enzyme compositions comprise an accessory enzyme. An accessory enzyme can have the same or similar function or a different function as an enzyme or enzymes in the core set of enzymes. These enzymes have been described elsewhere herein, and can generally include cellulases, xylanases, ligninases, amylases, lipidases, or glucuronidases, for example. For example, some accessory enzymes can include enzymes that when contacted with biomass in a reaction, allow for an increase in the activity of enzymes (e.g., hemicellulases) in the multi-enzyme composition. An accessory enzyme or enzyme mix may be composed of enzymes from (1) commercial suppliers; (2) cloned genes expressing enzymes; (3) complex broth (such as that resulting from growth of a microbial strain in media, wherein the strains secrete proteins and enzymes into the media); (4) cell lysates of strains grown as in (3); and, (5) plant material expressing enzymes capable of degrading lignocellulose. In some aspects, the accessory enzyme is a glucoamylase, a pectinase, or a ligninase.

As used herein, a ligninase is an enzyme that can hydrolyze or break down the structure of lignin polymers, including lignin peroxidases, manganese peroxidases, laccases, and other enzymes described in the art known to depolymerize or otherwise break lignin polymers. Also included are enzymes capable of hydrolyzing bonds formed between hemicellulosic sugars (notably arabinose) and lignin.

The multi-enzyme compositions, in some aspects, comprise a biomass comprising microorganisms or a crude fermentation product of microorganisms. A crude fermentation product refers to the fermentation broth which has been separated from the microorganism biomass (by filtration, for example). In general, the microorganisms are grown in fermenters, optionally centrifuged or filtered to remove biomass, and optionally concentrated, formulated, and dried to produce an enzyme(s) or a multi-enzyme composition that is a crude fermentation product. In other aspects, enzyme(s) or multi-enzyme compositions produced by the microorganism (including a genetically modified microorganism as described below) are subjected to one or more purification steps, such as ammonium sulfate precipitation, chromatography, and/or ultrafiltration, which result in a partially purified or purified enzyme(s). If the microorganism has been genetically modified to express the enzyme(s), the enzyme(s) will include recombinant enzymes. If the genetically modified microorganism also naturally expresses the enzyme(s) or other enzymes useful for lignocellulosic saccharification or any other useful application mentioned herein, the enzyme(s) may include both naturally occurring and recombinant enzymes.

Another aspect of the present invention relates to a composition comprising at least about 500 ng, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an isolated protein comprising any of the proteins or homologues, variants, or fragments thereof discussed herein. Such a composition of the present invention may include any carrier with which the protein is associated by virtue of the protein preparation method, a protein purification method, or a preparation of the protein for use in any method according to the present invention. For example, such a carrier can include any suitable buffer, extract, or medium that is suitable for combining with the protein of the present invention so that the protein can be used in any method described herein according to the present invention.

In one aspect of the invention, one or more enzymes of the invention is bound to a solid support, i.e., an immobilized enzyme. As used herein, an immobilized enzyme includes immobilized isolated enzymes, immobilized microbial cells which contain one or more enzymes of the invention, other stabilized intact cells that produce one or more enzymes of the invention, and stabilized cell/membrane homogenates. Stabilized intact cells and stabilized cell/membrane homogenates include cells and homogenates from naturally occurring microorganisms expressing the enzymes of the invention and preferably, from genetically modified microorganisms as disclosed elsewhere herein. Thus, although methods for immobilizing enzymes are discussed below, it will be appreciated that such methods are equally applicable to immobilizing microbial cells and in such an aspect, the cells can be lysed, if desired.

A variety of methods for immobilizing an enzyme are disclosed in Industrial Enzymology 2nd Ed., Godfrey, T. and West, S. Eds., Stockton Press, New York, N.Y., 1996, pp. 267-272; Immobilized Enzymes, Chibata, I. Ed., Halsted Press, New York, N.Y., 1978; Enzymes and Immobilized Cells in Biotechnology, Laskin, A. Ed., Benjamin/Cummings Publishing Co., Inc., Menlo Park, Calif., 1985; and Applied Biochemistry and Bioengineering, Vol. 4, Chibata, I. and Wingard, Jr., L. Eds, Academic Press, New York, N.Y., 1983.

Further aspects of the present invention include nucleic acid molecules that encode a protein of the present invention, as well as homologues, variants, or fragments of such nucleic acid molecules. A nucleic acid molecule of the present invention includes a nucleic acid molecule comprising, consisting essentially of, or consisting of, a nucleic acid sequence encoding any of the isolated proteins disclosed herein, including a fragment or a homologue or variant of such proteins, described above. Nucleic acid molecules can include a nucleic acid sequence that encodes a fragment of a protein that does not have biological activity, and can also include portions of a gene or polynucleotide encoding the protein that are not part of the coding region for the protein (e.g., introns or regulatory regions of a gene encoding the protein). Nucleic acid molecules can include a nucleic acid sequence that is useful as a probe or primer (oligonucleotide sequences).

In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule, and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. An isolated nucleic acid molecule of the present invention can be isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a protein of the present invention or to form stable hybrids under stringent conditions with natural gene isolates. An isolated nucleic acid molecule can include degeneracies. As used herein, nucleotide degeneracy refers to the phenomenon that one amino acid can be encoded by different nucleotide codons. Thus, the nucleic acid sequence of a nucleic acid molecule that encodes a protein of the present invention can vary due to degeneracies. It is noted that a nucleic acid molecule of the present invention is not required to encode a protein having protein activity. A nucleic acid molecule can encode a truncated, mutated or inactive protein, for example. In addition, nucleic acid molecules of the invention are useful as probes and primers for the identification, isolation and/or purification of other nucleic acid molecules. If the nucleic acid molecule is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

According to the present invention, reference to a gene includes all nucleic acid sequences related to a natural (i.e. wild-type) gene, such as regulatory regions that control production of the protein encoded by that gene (such as, but not limited to, transcription, translation or post-translation control regions) as well as the coding region itself. In another aspect, a gene can be a naturally occurring allelic variant that includes a similar but not identical sequence to the nucleic acid sequence encoding a given protein. Allelic variants have been previously described above. Genes can include or exclude one or more introns or any portions thereof or any other sequences or which are not included in the cDNA for that protein. The phrases “nucleic acid molecule” and “gene” can be used interchangeably when the nucleic acid molecule comprises a gene as described above.

Preferably, an isolated nucleic acid molecule of the present invention is produced using recombinant DNA technology (e.g., polymerase chain reaction (PCR) amplification, cloning, etc.) or chemical synthesis. Isolated nucleic acid molecules include any nucleic acid molecules and homologues or variants thereof that are part of a gene described herein and/or that encode a protein described herein, including, but not limited to, natural allelic variants and modified nucleic acid molecules (homologues or variants) in which nucleotides have been inserted, deleted, substituted, and/or inverted in such a manner that such modifications provide the desired effect on protein biological activity or on the activity of the nucleic acid molecule. Allelic variants and protein homologues or variants (e.g., proteins encoded by nucleic acid homologues or variants) have been discussed in detail above.

A nucleic acid molecule homologue or variant (i.e., encoding a homologue or variant of a protein of the present invention) can be produced using a number of methods known to those skilled in the art (see, for example, Sambrook et al.). For example, nucleic acid molecules can be modified using a variety of techniques including, but not limited to, by classic mutagenesis and recombinant DNA techniques (e.g., site-directed mutagenesis, chemical treatment, restriction enzyme cleavage, ligation of nucleic acid fragments and/or PCR amplification), or synthesis of oligonucleotide mixtures and ligation of mixture groups to “build” a mixture of nucleic acid molecules and combinations thereof. Another method for modifying a recombinant nucleic acid molecule encoding a protein is gene shuffling (i.e., molecular breeding) (See, for example, U.S. Pat. No. 5,605,793 to Stemmer; Minshull and Stemmer; 1999, Curr. Opin. Chem. Biol. 3:284-290; Stemmer, 1994, P.N.A.S. USA 91:10747-10751). This technique can be used to efficiently introduce multiple simultaneous changes in the protein. Nucleic acid molecule homologues or variants can be selected by hybridization with a gene or polynucleotide, or by screening for the function of a protein encoded by a nucleic acid molecule (i.e., biological activity).

The minimum size of a nucleic acid molecule of the present invention is a size sufficient to encode a protein (including a fragment, homologue, or variant of a full-length protein) having biological activity, sufficient to encode a protein comprising at least one epitope which binds to an antibody, or sufficient to form a probe or oligonucleotide primer that is capable of forming a stable hybrid with the complementary sequence of a nucleic acid molecule encoding a natural protein (e.g., under moderate, high, or high stringency conditions). As such, the size of the nucleic acid molecule encoding such a protein can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein encoding sequence, a nucleic acid sequence encoding a full-length protein (including a gene), including any length fragment between about 20 nucleotides and the number of nucleotides that make up the full length cDNA encoding a protein, in whole integers (e.g., 20, 21, 22, 23, 24, 25 . . . nucleotides), or multiple genes, or portions thereof.

The phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

In one aspect, the polynucleotide probes or primers of the invention are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or, ³²P) enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate such as: artificial membranes, organic supports, biopolymer supports and inorganic supports.

One aspect of the present invention relates to a recombinant nucleic acid molecule which comprises the isolated nucleic acid molecule described above which is operatively linked to at least one expression control sequence. More particularly, according to the present invention, a recombinant nucleic acid molecule typically comprises a recombinant vector and any one or more of the isolated nucleic acid molecules as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remains separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one aspect, a recombinant vector used in a recombinant nucleic acid molecule of the present invention is an expression vector. As used herein, the phrase “expression vector” is used to refer to a vector that is suitable for production of an encoded product (e.g., a protein of interest, such as an enzyme of the present invention). In this aspect, a nucleic acid sequence encoding the product to be produced (e.g., the protein or homologue or variant thereof) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

Typically, a recombinant nucleic acid molecule includes at least one nucleic acid molecule of the present invention operatively linked to one or more expression control sequences (e.g., transcription control sequences or translation control sequences). As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced. Transcription control sequences may also include any combination of one or more of any of the foregoing.

Recombinant nucleic acid molecules of the present invention can also contain additional regulatory sequences, such as translation regulatory sequences, origins of replication, and other regulatory sequences that are compatible with the recombinant cell. In one aspect, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another aspect, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

According to the present invention, the term “transfection” is generally used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants and describes an inherited change due to the acquisition of exogenous nucleic acids by the microorganism that is essentially synonymous with the term “transfection.” Transfection techniques include, but are not limited to, transformation, particle bombardment, electroporation, microinjection, lipofection, adsorption, infection and protoplast fusion.

One or more recombinant molecules of the present invention can be used to produce an encoded product (e.g., a protein) of the present invention. In one aspect, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., filamentous fungi or yeast or mushrooms), algal, plant, insect, or animal cell that can be transfected. Host cells can be either untransfected cells or cells that are already transfected with at least one other recombinant nucleic acid molecule.

Suitable cells (e.g., a host cell or production organism) may include any microorganism (e.g., a bacterium, a protist, an alga, a fungus, or other microbe), and is preferably a bacterium, a yeast or a filamentous fungus. Suitable bacterial genera include, but are not limited to, Escherichia, Bacillus, Lactobacillus, Pseudomonas and Streptomyces. Suitable bacterial species include, but are not limited to, Escherichia coli, Bacillus subtilis, Bacillus licheniformis, Bacillus stearothermophilus, Lactobacillus brevis, Pseudomonas aeruginosa and Streptomyces lividans. Suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Hansenula polymorpha, Pichia pastoris, P. canadensis, Kluyveromyces marxianus and Phaffia rhodozyma.

Suitable fungal genera include, but are not limited to, Chrysosporium, Thielavia, Neurospora, Aureobasidium, Filibasidium, Piromyces, Corynascus, Cryptococcus, Acremonium, Tolypocladium, Scytalidium, Schizophyllum, Sporotrichum, Penicillium, Gibberella, Myceliophthora, Mucor, Aspergillus, Fusarium, Humicola, and Trichoderma, and anamorphs and teleomorphs thereof. Suitable fungal species include, but are not limited to, Aspergillus niger, Aspergillus oryzae, Aspergillus nidulans, Aspergillus japonicus, Absidia coerulea, Rhizopus oryzae, Myceliophthora thermophila, Neurospora crassa, Neurospora intermedia, Trichoderma reesei, Trichoderma longibrachiatum, Penicillium canescens, Penicillium solitum, Penicillium funiculosum, Myceliophthora thermophila, Acremonium alabamense, Thielavia terrestris, Sporotrichum thermophile, Sporotrichum cellulophilum, Chaetomium globosum, Corynascus heterothallicus, and Talaromyces flavus. In another aspect, a while (low cellulose) strain is sued. In one aspect, the host cell is a fungal cell of Strain C1 (VKM F-3500 D) or a mutant strain derived therefrom (e.g., UV13-6 (Accession No. VKM F-3632 D); NG7C-19 (Accession No. VKM F-3633 D); UV18-25 (VKM F-3631D), W1L (CBS122189), or W1L#100L (CBS122190)). The C1 strain was initially classified as Myceliophthora thermophila based on morphological characteristics and was subsequently reclassified as M. thermophila based on genetic tests. Host cells can be either untransfected cells or cells that are already transfected with at least one other recombinant nucleic acid molecule. Additional aspects of the present invention include any of the genetically modified cells described herein.

In another aspect, suitable host cells include insect cells (most particularly Drosophila melanogaster cells, Spodoptera frugiperda Sf9 and Sf21 cells and Trichoplusia High-Five cells), nematode cells (particularly C. elegans cells), avian cells, amphibian cells (particularly Xenopus laevis cells), reptilian cells, and mammalian cells (most particularly human, simian, canine, rodent, bovine, or sheep cells, e.g. NIH3T3, CHO (Chinese hamster ovary cell), COS, VERO, BHK, HEK, and other rodent or human cells).

In one aspect, one or more protein(s) expressed by an isolated nucleic acid molecule of the present invention are produced by culturing a cell that expresses the protein (i.e., a recombinant cell or recombinant host cell) under conditions effective to produce the protein. In some instances, the protein may be recovered, and in others, the cell may be harvested in whole, either of which can be used in a composition.

Microorganisms used in the present invention (including recombinant host cells or genetically modified microorganisms) are cultured in an appropriate fermentation medium. An appropriate, or effective, fermentation medium refers to any medium in which a cell of the present invention, including a genetically modified microorganism (described below), when cultured, is capable of expressing enzymes useful in the present invention and/or of catalyzing the production of sugars from lignocellulosic biomass. The microorganisms can be cultured by any fermentation process which includes, but is not limited to, batch, fed-batch, cell recycle, and continuous fermentation. In general the fungal strains are grown in fermenters, optionally centrifuged or filtered to remove biomass, and optionally concentrated, formulated, and dried to produce an enzyme(s) or a multi-enzyme composition that is a crude fermentation product. Particularly suitable conditions for culturing filamentous fungi are described, for example, in U.S. Pat. No. 6,015,707 and U.S. Pat. No. 6,573,086, supra.

Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential precipitation or solubilization.

Proteins of the present invention are preferably retrieved, obtained, and/or used in “substantially pure” form. As used herein, “substantially pure” refers to a purity that allows for the effective use of the protein in any method according to the present invention. For a protein to be useful in any of the methods described herein or in any method utilizing enzymes of the types described herein according to the present invention, it is substantially free of contaminants, other proteins and/or chemicals that might interfere or that would interfere with its use in a method disclosed by the present invention (e.g., that might interfere with enzyme activity), or that at least would be undesirable for inclusion with a protein of the present invention (including homologues and variants) when it is used in a method disclosed by the present invention (described in detail below). Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the protein of interest is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites), modification of nucleic acid molecules to correspond to the codon usage of the host cell, and deletion of sequences that destabilize transcripts.

Another aspect of the present invention relates to a genetically modified microorganism that has been transfected with one or more nucleic acid molecules of the present invention. As used herein, a genetically modified microorganism can include a genetically modified bacterium, alga, yeast, filamentous fungus, or other microbe. Such a genetically modified microorganism has a genome which is modified (i.e., mutated or changed) from its normal (i.e., wild-type or naturally occurring) form such that the desired result is achieved (i.e., increased or modified activity and/or production of at least one enzyme or a multi-enzyme composition for the conversion of lignocellulosic material to fermentable sugars). Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor Labs Press or Molecular Cloning: A Laboratory Manual, third edition (Sambrook and Russell, 2001), (jointly referred to herein as “Sambrook”). A genetically modified microorganism can include a microorganism in which nucleic acid molecules have been inserted, deleted or modified (i.e., mutated; e.g., by insertion, deletion, substitution, and/or inversion of nucleotides), in such a manner that such modifications provide the desired effect within the microorganism.

In one aspect, a genetically modified microorganism can endogenously contain and express an enzyme or a multi-enzyme composition and the genetic modification can be a genetic modification of one or more of such endogenous enzymes, whereby the modification has some effect on the amount and/or quality of enzyme mixtures produced by the organism of the microorganism (e.g., increased expression of the protein by introduction of promoters or other expression control sequences, or modification of the coding region by homologous recombination to increase the activity of the encoded protein).

In another aspect, a genetically modified microorganism can endogenously contain and express an enzyme for the catalysis of oxidation-reduction reactions, and the genetic modification can be an introduction of at least one exogenous nucleic acid sequence (e.g., a recombinant nucleic acid molecule), wherein the exogenous nucleic acid sequence encodes at least one additional enzyme useful for the catalysis of oxidation-reduction reactions and/or a protein that improves the efficiency of the target enzyme. In this aspect of the invention, the microorganism can also have at least one modification to a gene or genes comprising its endogenous enzyme(s) for the catalysis of oxidation-reduction reactions or an enzyme to aid in the conversion of lignocellulosic material.

In yet another aspect, the genetically modified microorganism does not necessarily endogenously (naturally) contain an enzyme for the catalysis of oxidation-reduction reactions, but is genetically modified to introduce at least one recombinant nucleic acid molecule encoding at least one enzyme or a multiplicity of enzymes for the catalysis of oxidation-reduction reactions. Such a microorganism can be used in a method of the invention, or as a production microorganism for crude fermentation products, partially purified recombinant enzymes, and/or purified recombinant enzymes, any of which can then be used in a method of the present invention.

Once the proteins (enzymes) are expressed in a host cell, a cell extract that contains the activity to test can be generated. For example, a lysate from the host cell is produced, and the supernatant containing the activity is harvested and/or the activity can be isolated from the lysate. In the case of cells that secrete enzymes into the culture medium, the culture medium containing them can be harvested, and/or the activity can be purified from the culture medium. The extracts/activities prepared in this way can be tested using assays known in the art.

The present invention is not limited to fungi and also contemplates genetically modified organisms such as algae, bacterial, and plants transformed with one or more nucleic acid molecules of the invention. The plants may be used for production of the enzymes, and/or as the lignocellulosic material used as a substrate in the methods of the invention. Methods to generate recombinant plants are known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized method for introducing an expression vector into plants is based on the natural transformation system of Agrobacterium. See, for example, Horsch et al., Science 227:1229 (1985). Descriptions of Agrobacterium vector systems and methods for Agrobacterium-mediated gene transfer are provided by numerous references, including Gruber et al., supra, Miki et al., supra, Moloney et al., Plant Cell Reports 8:238 (1989), and U.S. Pat. Nos. 4,940,838 and 5,464,763.

Another generally applicable method of plant transformation is microprojectile-mediated transformation, see e.g., Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion have been used to introduce expression vectors into plants. Deshayes et al., EMBO J., 4:2731 (1985), Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VII^{th} International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

Some aspects of the present invention include genetically modified organisms comprising at least one nucleic acid molecule encoding at least one enzyme of the present invention, in which the activity of the enzyme is downregulated. The downregulation may be achieved, for example, by introduction of inhibitors (chemical or biological) of the enzyme activity, by manipulating the efficiency with which those nucleic acid molecules are transcribed, the efficiency with which the resultant transcripts are translated, and the efficiency of post-translational modifications, or by “knocking out” the endogenous copy of the gene. A “knock out” of a gene refers to a molecular biological technique by which the gene in the organism is made inoperative, so that the expression of the gene is substantially reduced or eliminated. Alternatively, in some aspects the activity of the enzyme may be upregulated. The present invention also contemplates downregulating activity of one or more enzymes while simultaneously upregulating activity of one or more enzymes to achieve the desired outcome.

Proteins of the present invention, at least one protein of the present invention, compositions comprising such protein(s) of the present invention, and multi-enzyme compositions (examples of which are described above) may be used in any method where it is desirable to hydrolyze glycosidic linkages in lignocellulosic material, or any other method wherein enzymes of the same or similar function are useful.

In one aspect, the present invention includes the use of at least one protein of the present invention, compositions comprising at least one protein of the present invention, or multi-enzyme compositions in methods for hydrolyzing lignocellulose and the generation of fermentable sugars therefrom. In one aspect, the method comprises contacting the lignocellulosic material with an effective amount of one or more proteins of the present invention, composition comprising at least one protein of the present invention, or a multi-enzyme composition, whereby at least one fermentable sugar is produced (liberated). The lignocellulosic material may be partially or completely degraded to fermentable sugars. Economical levels of degradation at commercially viable costs are contemplated.

Typically, the amount of enzyme or enzyme composition contacted with the lignocellulose will depend upon the amount of glucan present in the lignocellulose. In some aspects, the amount of enzyme or enzyme composition contacted with the lignocellulose may be from about 0.1 to about 200 mg enzyme or enzyme composition per gram of glucan; in other aspects, from about 3 to about 20 mg enzyme or enzyme composition per gram of glucan. The invention encompasses the use of any suitable or sufficient amount of enzyme or enzyme composition between about 0.1 mg and about 200 mg enzyme per gram glucan, in increments of 0.05 mg (i.e., 0.1 mg, 0.15 mg, 0.2 mg . . . 199.9 mg, 199.95 mg, 200 mg).

In a further aspect, the invention provides a method for degrading DDG, preferably, but not limited to, DDG derived from corn, to sugars. The method comprises contacting the DDG with a protein of the present invention, a composition comprising at least one protein of the present invention, or a multi-enzyme composition. In certain aspects, at least 10% of fermentable sugars are liberated. In other aspect, the at least 15% of the sugars are liberated, or at least 20% of the sugars are liberated, or at least 23% of the sugars are liberated, or at least 24% of the sugars are liberated, or at least 25% of the sugars are liberated, or at least 26% of the sugars are liberated, or at least 27% of the sugars are liberated, or at least 28% of the sugars are liberated.

In another aspect, the invention provides a method for producing fermentable sugars comprising cultivating a genetically modified microorganism of the present invention in a nutrient medium comprising a lignocellulosic material, whereby fermentable sugars are produced.

Also provided are methods that comprise further contacting the lignocellulosic material with at least one accessory enzyme. Accessory enzymes have been described elsewhere herein. The accessory enzyme or enzymes may be added at the same time, prior to, or following the addition of a protein of the present invention, a composition comprising at least one protein of the present invention, or a multi-enzyme composition, or can be expressed (endogenously or overexpressed) in a genetically modified microorganism used in a method of the invention. When added simultaneously, the protein of the present invention, a composition comprising at least one protein of the present invention, or a multi-enzyme composition will be compatible with the accessory enzymes selected. When the enzymes are added following the treatment with the protein of the present invention, a composition comprising at least one protein of the present invention, or a multi-enzyme composition, the conditions (such as temperature and pH) may be altered to those optimal for the accessory enzyme before, during, or after addition of the accessory enzyme. Multiple rounds of enzyme addition are also encompassed. The accessory enzyme may also be present in the lignocellulosic material itself as a result of genetically modifying the plant. The nutrient medium used in a fermentation can also comprise one or more accessory enzymes.

In some aspects, the method comprises a pretreatment process. In general, a pretreatment process will result in components of the lignocellulose being more accessible for downstream applications or so that it is more digestible by enzymes following treatment in the absence of hydrolysis. The pretreatment can be a chemical, physical or biological pretreatment. The lignocellulose may have been previously treated to release some or all of the sugars, as in the case of DDG. Physical treatments, such as grinding, boiling, freezing, milling, vacuum infiltration, and the like may also be used with the methods of the invention. In one aspect, the heat treatment comprises heating the lignocellulosic material to 121° C. for 15 minutes. A physical treatment such as milling can allow a higher concentration of lignocellulose to be used in the methods of the invention. A higher concentration refers to about 20%, up to about 25%, up to about 30%, up to about 35%, up to about 40%, up to about 45%, or up to about 50% lignocellulose. The lignocellulose may also be contacted with a metal ion, ultraviolet light, ozone, and the like. Additional pretreatment processes are known to those skilled in the art, and can include, for example, organosolv treatment, steam explosion treatment, lime impregnation with steam explosion treatment, hydrogen peroxide treatment, hydrogen peroxide/ozone (peroxone) treatment, acid treatment, dilute acid treatment, and base treatment, including ammonia fiber explosion (AFEX) technology. Details on pretreatment technologies and processes can be found in Wyman et al., Bioresource Tech. 96:1959 (2005); Wyman et al., Bioresource Tech. 96:2026 (2005); Hsu, “Pretreatment of biomass” In Handbook on Bioethanol: Production and Utilization, Wyman, Taylor and Francis Eds., p. 179-212 (1996); and Mosier et al., Bioresource Tech. 96:673 (2005).

In some aspects, the methods may be performed one or more times in whole or in part. That is, one may perform one or more pretreatments, followed by one or more reactions with a protein of the present invention, composition or product of the present invention and/or accessory enzyme. The enzymes may be added in a single dose, or may be added in a series of small doses. Further, the entire process may be repeated one or more times as necessary. Therefore, one or more additional treatments with heat and enzymes are contemplated.

The methods described above result in the production of fermentable sugars. During, or subsequent to the methods described, the fermentable sugars may be recovered and/or purified by any method known in the art. The sugars can be subjected to further processing; e.g., they can also be sterilized, for example, by filtration.

In an additional aspect, the invention provides a method for producing an organic substance, comprising saccharifying a lignocellulosic material with an effective amount of a protein of the present invention or a composition comprising at least one protein of the present invention, fermenting the saccharified lignocellulosic material obtained with one or more microorganisms, and recovering the organic substance from the fermentation. Sugars released from biomass can be converted to useful fermentation products including but not limited to amino acids, vitamins, pharmaceuticals, animal feed supplements, specialty chemicals, chemical feedstocks, plastics, solvents, fuels, or other organic polymers, lactic acid, and ethanol, including fuel ethanol. Specific products that may be produced by the methods of the invention include, but not limited to, biofuels (including ethanol); lactic acid; plastics; specialty chemicals; organic acids, including citric acid, succinic acid, itaconic and maleic acid; solvents; animal feed supplements; pharmaceuticals; vitamins; amino acids, such as lysine, methionine, tryptophan, threonine, and aspartic acid; industrial enzymes, such as proteases, cellulases, amylases, glucanases, lactases, lipases, lyases, oxidoreductases, and transferases; and chemical feedstocks. The methods of the invention are also useful to generate feedstocks for fermentation by fermenting microorganisms. In one aspect, the method further comprises the addition of at least one fermenting organism.

As used herein, “fermenting organism” refers to an organism capable of fermentation, such as bacteria and fungi, including yeast. Such feedstocks have additional nutritive value above the nutritive value provided by the liberated sugars.

In some aspects the invention comprises, but is not limited to methods for oxidoreductases in the biofuel industry, such as lignin degradation.

In some aspects the invention comprises, but is not limited to additional methods for oxidoreductases, such as biosensors; diagnostic (analytical) kits; effective additives for refolding immunoglobulin-folded proteins in vitro; bleaching cotton; polymerizing phenols and aromatic amines; asymmetric syntheses of amino acids, steroids, pharmaceuticals and other fine chemicals; biocatalysis; pollution control, and oxygenation of hydrocarbons; treatment of industrial waste waters (detoxification); soil detoxification; manufacturing of adhesives, computer chips, car parts, and linings of drums and cans; whitening the skin/hair/teeth; and stimulating the immune system.

Exemplary methods according to the invention are presented below. Examples of the methods described above may also be found in the following references: Trichoderma & Gliocladium, Volume 2, Enzymes, biological control and commercial applications, Editors: Gary E. Harman, Christian P. Kubicek, Taylor & Francis Ltd. 1998, 393 (in particular, chapters 14, 15 and 16); Helmut Uhlig, Industrial enzymes and their applications, Translated and updated by Elfriede M. Linsmaier-Bednar, John Wiley & Sons, Inc 1998, p. 454 (in particular, chapters 5.1, 5.2, 5.3, 5.4, 5.5, 5.6, 5.7, 5.9, 5.10, 5.11, and 5.13). For saccharification applications: Hahn-Hägerdal, B., Galbe, M., Gorwa-Grauslund, M. F. Lidén, Zacchi, G. Bio-ethanol—the fuel of tomorrow from the residues of today, Trends in Biotechnology, 2006, 24 (12), 549-556; Mielenz, J. R. Ethanol production from biomass: technology and commercialization status, Current Opinion in Microbiology, 2001, 4, 324-329; Himmel, M. E., Ruth, M. F., Wyman, C. E., Cellulase for commodity products from cellulosic biomass, Current Opinion in Biotechnology, 1999, 10, 358-364; Sheehan, J., Himmel, M. Enzymes, energy, and the environment: a strategic perspective on the U.S. Department of Energy's Research and Development Activities for Bioethanol, Biotechnology Progress. 1999, 15, 817-827. For textile processing applications: Galante, Y. M., Formantici, C., Enzyme applications in detergency and in manufacturing industries, Current Organic Chemistry, 2003, 7, 1399-1422. For pulp and paper applications: Bajpai, P., Bajpai, P. K Deinking with enzymes: a review. TAPPI Journal, 1998, 81(12), 111-117; Viikari, L., Pere, J., Suurnäkki, A., Oksanen, T., Buchert, J. Use of cellulases in pulp and paper applications. In: “Carbohydrates from Trichoderma reesei and other microorganisms. Structure, Biochemistry, Genetics and Applications.” Editors: Mark Claessens, Wim Nerinckx, and Kathleen Piens, The Royal Society of Chemistry 1998, 245-254. For food and beverage applications: Roller, S., Dea, I. C. M. Biotechnology in the production and modification of biopolymers for foods, Critical Reviews in Biotechnology, 1992, 12(3), 261-277.

Additional references include, U.S. Pat. No. 5,529,926; U.S. Pat. No. 6,746,679; U.S. Pat. No. 7,732,178; U.S. Pat. No. 6,660,128; U.S. Pat. No. 6,093,436; U.S. Pat. No. 5,691,193; U.S. patent No. As used herein, reference to an isolated protein or polypeptide in the present invention, including any of the enzymes disclosed herein, includes full-length proteins and their glycosylated or otherwise modified forms, fusion proteins, or any fragment or homologue or variant of such a protein. More specifically, an isolated protein, such as an enzyme according to the present invention, is a protein (including a polypeptide or peptide) that has been removed from its natural milieu (i.e., that has been subject to human manipulation) and can include purified proteins, partially purified proteins, recombinantly produced proteins, synthetically produced proteins, proteins complexed with lipids, soluble proteins, and isolated proteins associated with other proteins, for example. As such, “isolated” does not reflect the extent to which the protein has been purified. Preferably, an isolated protein of the present invention is produced recombinantly. In addition, and by way of example, a “M. thermophila protein” or “M. thermophila enzyme” refers to a protein (generally including a homologue or variant of a naturally occurring protein) from Myceliophthora thermophila or to a protein that has been otherwise produced from the knowledge of the structure (e.g., sequence) and perhaps the function of a naturally occurring protein from Myceliophthora thermophila. In other words, a M. thermophila protein includes any protein that has substantially similar structure and function of a naturally occurring M. thermophila protein or that is a biologically active (i.e., has biological activity) homologue or variant of a naturally occurring protein from M. thermophila as described in detail herein. As such, a M. thermophila protein can include purified, partially purified, recombinant, mutated/modified and synthetic proteins.

According to the present invention, the terms “modification,” “mutation,” and “variant” can be used interchangeably, particularly with regard to the modifications/mutations to the amino acid sequence of a M. thermophila protein (or nucleic acid sequences) described herein. An isolated protein according to the present invention can be isolated from its natural source, produced recombinantly or produced synthetically.

According to the present invention, the terms “modification” and “mutation” can be used interchangeably, particularly with regard to the modifications/mutations to the primary amino acid sequences of a protein or peptide (or nucleic acid sequences) described herein. The term “modification” can also be used to describe post-translational modifications to a protein or peptide including, but not limited to, methylation, farnesylation, carboxymethylation, geranyl geranylation, glycosylation, phosphorylation, acetylation, myristoylation, prenylation, palmitation, and/or amidation. Modification can also include the cleavage of a signal peptide, or methionine, or other portions of the peptide that require cleavage to generate the mature peptide.

As used herein, the terms “homologue” or “variants” are used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide), insertions and/or substitutions; changes in stereochemistry of one or a few atoms; and/or minor derivatizations, including but not limited to for example: methylation, glycosylation and phosphorylation. A homologue or variant can have either enhanced, decreased, or substantially similar properties as compared to the naturally occurring protein or peptide. A homologue or variant can include an agonist of a protein or an antagonist of a protein.

Homologues or variants can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Homologous can also be the result of a gene duplication and rearrangement, resulting in a different location. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues or variants can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications of a protein, such as in a homologue or variant, may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

According to the present invention, an isolated protein, including a biologically active homologue, variant, or fragment thereof, has at least one characteristic of biological activity of a wild-type, or naturally occurring, protein. As discussed above, in general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). The biological activity of a protein of the present invention can include an enzyme activity (catalytic activity and/or substrate binding activity), such as oxidases, oxygenases, monoxygenases, Baeyer-Villiger monooxygenases, dioxygenases, peroxidases, dehydrogenases, reductases that catalyze an oxidation-reduction reaction or any other activity disclosed herein. Specific biological activities of the proteins disclosed herein are described in detail above and in the Examples. Methods of detecting and measuring the biological activity of a protein of the invention include, but are not limited to, the assays described in the Examples section below. Such assays include, but are not limited to, measurement of enzyme activity (e.g., catalytic activity), measurement of substrate binding, and the like. It is noted that an isolated protein of the present invention (including homologues or variants) is not required to have a biological activity such as catalytic activity. A protein can be a truncated, mutated or inactive protein, or lack at least one activity of the wild-type enzyme, for example. Inactive proteins may be useful in some screening assays, for example, or for other purposes such as antibody production.

Methods to measure protein expression levels of a protein according to the invention include, but are not limited to: western blotting, immunocytochemistry, flow cytometry or other immunologic-based assays; assays based on a property of the protein including but not limited to, ligand binding or interaction with other protein partners.

Many of the enzymes and proteins of the present invention may be desirable targets for modification and use in the processes described herein. These proteins have been described in terms of function and amino acid sequence (and nucleic acid sequence encoding the same) of representative wild-type proteins. In one aspect of the invention, homologues or variants of a given protein (which can include related proteins from other organisms or modified forms of the given protein) are encompassed for use in the invention. Homologues or variants of a protein encompassed by the present invention can comprise, consist essentially of, or consist of, in one aspect, an amino acid sequence that is at least about 35% identical, and more preferably at least about 40% identical, and more preferably at least about 45% identical, and more preferably at least about 50% identical, and more preferably at least about 55% identical, and more preferably at least about 60% identical, and more preferably at least about 65% identical, and more preferably at least about 70% identical, and more preferably at least about 75% identical, and more preferably at least about 80% identical, and more preferably at least about 85% identical, and more preferably at least about 90% identical, and more preferably at least about 95% identical, and more preferably at least about 96% identical, and more preferably at least about 97% identical, and more preferably at least about 98% identical, and more preferably at least about 99% identical, or any percent identity between 35% and 99%, in whole integers (i.e., 36%, 37%, etc.), to an amino acid sequence disclosed herein that represents the amino acid sequence of an enzyme or protein according to the invention (including a biologically active domain of a full-length protein). Preferably, the amino acid sequence of the homologue or variant has a biological activity of the wild-type or reference protein or of a biologically active domain thereof (e.g., a catalytic domain). When denoting mutation positions, the amino acid position of the wild-type is typically used. The wild-type can also be referred to as the “parent.” Additionally, any generation before the variant at issue can be a parent.

In one aspect, a protein of the present invention comprises, consists essentially of, or consists of an amino acid sequence that, alone or in combination with other characteristics of such proteins disclosed herein, is less than 100% identical to a known amino acid sequence (i.e., a homologue or variant). For example, a protein of the present invention can be less than 100% identical, in combination with being at least about 35% identical, to a given disclosed sequence. In another aspect of the invention, a homologue or variant according to the present invention has an amino acid sequence that is less than about 99% identical to any of such amino acid sequences, and in another aspect, is less than about 98% identical to any of such amino acid sequences, and in another aspect, is less than about 97% identical to any of such amino acid sequences, and in another aspect, is less than about 96% identical to any of such amino acid sequences, and in another aspect, is less than about 95% identical to any of such amino acid sequences, and in another aspect, is less than about 94% identical to any of such amino acid sequences, and in another aspect, is less than about 93% identical to any of such amino acid sequences, and in another aspect, is less than about 92% identical to any of such amino acid sequences, and in another aspect, is less than about 91% identical to any of such amino acid sequences, and in another aspect, is less than about 90% identical to any of such amino acid sequences, and so on, in increments of whole integers.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402); (2) a BLAST 2 alignment (using the parameters described below); (3) PSI-BLAST with the standard default parameters (Position-Specific Iterated BLAST; and/or (4) CAZy homology determined using standard default parameters from the Carbohydrate Active EnZymes database (Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12) and/or applying a similar strategy using databases such as the Foly database (website: foly.esil.univ-mrs.fr) and the PeroxiBase (website: peroxibase.isb-sib.ch).

It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues or variants. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another aspect, a protein of the present invention, including a homologue or variant, includes a protein having an amino acid sequence that is sufficiently similar to a natural amino acid sequence that a nucleic acid sequence encoding the homologue or variant is capable of hybridizing under moderate, high or very high stringency conditions (described below) to (i.e., with) a nucleic acid molecule encoding the natural protein (i.e., to the complement of the nucleic acid strand encoding the natural amino acid sequence). Preferably, a homologue or variant of a protein of the present invention is encoded by a nucleic acid molecule comprising a nucleic acid sequence that hybridizes under low, moderate, or high stringency conditions to the complement of a nucleic acid sequence that encodes a protein comprising, consisting essentially of, or consisting of, an amino acid sequence represented by any of SEQ ID NO: Such hybridization conditions are described in detail below.

A nucleic acid sequence complement of nucleic acid sequence encoding a protein of the present invention refers to the nucleic acid sequence of the nucleic acid strand that is complementary to the strand which encodes the protein. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence such as the amino acid sequences of SEQ ID NO:. Methods to deduce a complementary sequence are known to those skilled in the art. It should be noted that since nucleic acid sequencing technologies are not entirely error-free, the sequences presented herein, at best, represent apparent sequences of the proteins of the present invention.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular aspects, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular aspects, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

The minimum size of a protein and/or homologue or variant of the present invention is a size sufficient to have biological activity or, when the protein is not required to have such activity, sufficient to be useful for another purpose associated with a protein of the present invention, such as for the production of antibodies that bind to a naturally occurring protein. In one aspect, the protein of the present invention is at least 20 amino acids in length, or at least about 25 amino acids in length, or at least about 30 amino acids in length, or at least about 40 amino acids in length, or at least about 50 amino acids in length, or at least about 60 amino acids in length, or at least about 70 amino acids in length, or at least about 80 amino acids in length, or at least about 90 amino acids in length, or at least about 100 amino acids in length, or at least about 125 amino acids in length, or at least about 150 amino acids in length, or at least about 175 amino acids in length, or at least about 200 amino acids in length, or at least about 250 amino acids in length, and so on up to a full length of each protein, and including any size in between in increments of one whole integer (one amino acid). There is no limit, other than a practical limit, on the maximum size of such a protein in that the protein can include a portion of a protein or a full-length protein, plus additional sequence (e.g., a fusion protein sequence), if desired.

The present invention also includes a fusion protein that includes a domain of a protein of the present invention (including a homologue or variant) attached to one or more fusion segments, which are typically heterologous in sequence to the protein sequence (i.e., different than protein sequence). Suitable fusion segments for use with the present invention include, but are not limited to, segments that can: enhance a protein's stability; provide other desirable biological activity; and/or assist with the purification of the protein (e.g., by affinity chromatography). A suitable fusion segment can be a domain of any size that has the desired function (e.g., imparts increased stability, solubility, action or biological activity; and/or simplifies purification of a protein). Fusion segments can be joined to amino and/or carboxyl termini of the domain of a protein of the present invention and can be susceptible to cleavage in order to enable straight-forward recovery of the protein. Fusion proteins are preferably produced by culturing a recombinant cell transfected with a fusion nucleic acid molecule that encodes a protein including the fusion segment attached to either the carboxyl and/or amino terminal end of a domain of a protein of the present invention. Accordingly, proteins of the present invention also include expression products of gene fusions (for example, used to overexpress soluble, active forms of the recombinant protein), of mutagenized genes (such as genes having codon modifications to enhance gene transcription and translation), and of truncated genes (such as genes having membrane binding modules removed to generate soluble forms of a membrane protein, or genes having signal sequences removed which are poorly tolerated in a particular recombinant host).

In one aspect of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived.

The present invention also provides enzyme combinations that break down or modify lignin material, reducing or preventing unwanted adsorption of other components of multi-enzyme compositions applied Such enzyme combinations or mixtures can include a multi-enzyme composition that contains at least one protein of the present invention in combination with one or more additional proteins of the present invention or one or more enzymes or other proteins from other microorganisms, plants, or similar organisms. Synergistic enzyme combinations and related methods are contemplated. The invention includes methods to identify the optimum ratios and compositions of enzymes with which to degrade each lignin and lignocellulosic material. These methods entail tests to identify the optimum enzyme composition and ratios for efficient conversion of any biomass substrate to its constituent sugars. The Examples below include assays that may be used to identify optimum ratios and compositions of enzymes with which to degrade lignocellulosic materials.

Any combination of the proteins disclosed herein is suitable for use in the multi-enzyme compositions of the present invention. It is to be understood that any of the enzymes described specifically herein can be combined with any one or more of the enzymes described herein or with any other available and suitable enzymes, to produce a multi-enzyme composition. The invention is not restricted or limited to the specific exemplary combinations listed below.

One or more components of a multi-enzyme composition (other than proteins of the present invention) can be obtained from or derived from a microbial, plant, or other source or combination thereof, and will contain enzymes capable of performing oxidation-reduction reactions. Examples of enzymes included in the multi-enzyme compositions of the invention include oxidases, oxygenases, monoxygenases, Baeyer-Villiger monooxygenases, dioxygenases, peroxidases, dehydrogenases, reductases that catalyze an oxidation-reduction reaction.

The multi-enzyme compositions of the invention can also include cellulases, hemicellulases (such as xylanases, including endoxylanases, exoxylanases, and β-xylosidases; mannanases, including endomannanases, exomannanases, and β-mannosidases), ligninases, amylases, glucuronidases, proteases, esterases (including ferulic acid esterase), lipases, glucosidases (such as β-glucosidase), and xyloglucanases.

While the multi-enzyme composition may contain many types of enzymes, mixtures comprising enzymes that increase or enhance sugar release from biomass are contemplated, which may include hemicellulases. In one aspect, the hemicellulase is selected from a xylanase, an arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xyloglucanase, or mixtures of any of these. In particular, the enzymes can include glucoamylase, β-xylosidase and/or β-glucosidase. Also preferred are mixtures comprising enzymes that are capable of degrading cell walls and releasing cellular contents.

The enzymes of the multi-enzyme composition can be provided by a variety of sources. In one aspect, the enzymes can be produced by growing organisms such as bacteria, algae, fungi, and plants which produce the enzymes naturally or by virtue of being genetically modified to express the enzyme or enzymes. In another aspect, at least one enzyme of the multi-enzyme composition is a commercially available enzyme.

In some aspects, the multi-enzyme compositions comprise an accessory enzyme. An accessory enzyme can have the same or similar function or a different function as an enzyme or enzymes in the core set of enzymes. These enzymes have been described elsewhere herein, and can generally include cellulases, xylanases, ligninases, amylases, lipidases, or glucuronidases, for example. For example, some accessory enzymes can include enzymes that when contacted with biomass in a reaction, allow for an increase in the activity of enzymes (e.g., hemicellulases) in the multi-enzyme composition. An accessory enzyme or enzyme mix may be composed of enzymes from (1) commercial suppliers; (2) cloned genes expressing enzymes; (3) complex broth (such as that resulting from growth of a microbial strain in media, wherein the strains secrete proteins and enzymes into the media); (4) cell lysates of strains grown as in (3); and, (5) plant material expressing enzymes capable of degrading lignocellulose. In some aspects, the accessory enzyme is a glucoamylase, a pectinase, or a ligninase.

As used herein, a ligninase is an enzyme that can hydrolyze or break down the structure of lignin polymers, including lignin peroxidases, manganese peroxidases, laccases, and other enzymes described in the art known to depolymerize or otherwise break lignin polymers. Also included are enzymes capable of hydrolyzing bonds formed between hemicellulosic sugars (notably arabinose) and lignin.

The multi-enzyme compositions, in some aspects, comprise a biomass comprising microorganisms or a crude fermentation product of microorganisms. A crude fermentation product refers to the fermentation broth which has been separated from the microorganism biomass (by filtration, for example). In general, the microorganisms are grown in fermenters, optionally centrifuged or filtered to remove biomass, and optionally concentrated, formulated, and dried to produce an enzyme(s) or a multi-enzyme composition that is a crude fermentation product. In other aspects, enzyme(s) or multi-enzyme compositions produced by the microorganism (including a genetically modified microorganism as described below) are subjected to one or more purification steps, such as ammonium sulfate precipitation, chromatography, and/or ultrafiltration, which result in a partially purified or purified enzyme(s). If the microorganism has been genetically modified to express the enzyme(s), the enzyme(s) will include recombinant enzymes. If the genetically modified microorganism also naturally expresses the enzyme(s) or other enzymes useful for lignocellulosic saccharification or any other useful application mentioned herein, the enzyme(s) may include both naturally occurring and recombinant enzymes.

Another aspect of the present invention relates to a composition comprising at least about 500 ng, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an isolated protein comprising any of the proteins or homologues, variants, or fragments thereof discussed herein. Such a composition of the present invention may include any carrier with which the protein is associated by virtue of the protein preparation method, a protein purification method, or a preparation of the protein for use in any method according to the present invention. For example, such a carrier can include any suitable buffer, extract, or medium that is suitable for combining with the protein of the present invention so that the protein can be used in any method described herein according to the present invention.

In one aspect of the invention, one or more enzymes of the invention is bound to a solid support, i.e., an immobilized enzyme. As used herein, an immobilized enzyme includes immobilized isolated enzymes, immobilized microbial cells which contain one or more enzymes of the invention, other stabilized intact cells that produce one or more enzymes of the invention, and stabilized cell/membrane homogenates. Stabilized intact cells and stabilized cell/membrane homogenates include cells and homogenates from naturally occurring microorganisms expressing the enzymes of the invention and preferably, from genetically modified microorganisms as disclosed elsewhere herein. Thus, although methods for immobilizing enzymes are discussed below, it will be appreciated that such methods are equally applicable to immobilizing microbial cells and in such an aspect, the cells can be lysed, if desired.

A variety of methods for immobilizing an enzyme are disclosed in Industrial Enzymology 2nd Ed., Godfrey, T. and West, S. Eds., Stockton Press, New York, N.Y., 1996, pp. 267-272; Immobilized Enzymes, Chibata, I. Ed., Halsted Press, New York, N.Y., 1978; Enzymes and Immobilized Cells in Biotechnology, Laskin, A. Ed., Benjamin/Cummings Publishing Co., Inc., Menlo Park, Calif., 1985; and Applied Biochemistry and Bioengineering, Vol. 4, Chibata, I. and Wingard, Jr., L. Eds, Academic Press, New York, N.Y., 1983.

Further aspects of the present invention include nucleic acid molecules that encode a protein of the present invention, as well as homologues, variants, or fragments of such nucleic acid molecules. A nucleic acid molecule of the present invention includes a nucleic acid molecule comprising, consisting essentially of, or consisting of, a nucleic acid sequence encoding any of the isolated proteins disclosed herein, including a fragment or a homologue or variant of such proteins, described above. Nucleic acid molecules can include a nucleic acid sequence that encodes a fragment of a protein that does not have biological activity, and can also include portions of a gene or polynucleotide encoding the protein that are not part of the coding region for the protein (e.g., introns or regulatory regions of a gene encoding the protein). Nucleic acid molecules can include a nucleic acid sequence that is useful as a probe or primer (oligonucleotide sequences).

In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule, and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. An isolated nucleic acid molecule of the present invention can be isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a protein of the present invention or to form stable hybrids under stringent conditions with natural gene isolates. An isolated nucleic acid molecule can include degeneracies. As used herein, nucleotide degeneracy refers to the phenomenon that one amino acid can be encoded by different nucleotide codons. Thus, the nucleic acid sequence of a nucleic acid molecule that encodes a protein of the present invention can vary due to degeneracies. It is noted that a nucleic acid molecule of the present invention is not required to encode a protein having protein activity. A nucleic acid molecule can encode a truncated, mutated or inactive protein, for example. In addition, nucleic acid molecules of the invention are useful as probes and primers for the identification, isolation and/or purification of other nucleic acid molecules. If the nucleic acid molecule is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

According to the present invention, reference to a gene includes all nucleic acid sequences related to a natural (i.e. wild-type) gene, such as regulatory regions that control production of the protein encoded by that gene (such as, but not limited to, transcription, translation or post-translation control regions) as well as the coding region itself. In another aspect, a gene can be a naturally occurring allelic variant that includes a similar but not identical sequence to the nucleic acid sequence encoding a given protein. Allelic variants have been previously described above. Genes can include or exclude one or more introns or any portions thereof or any other sequences or which are not included in the cDNA for that protein. The phrases “nucleic acid molecule” and “gene” can be used interchangeably when the nucleic acid molecule comprises a gene as described above.

Preferably, an isolated nucleic acid molecule of the present invention is produced using recombinant DNA technology (e.g., polymerase chain reaction (PCR) amplification, cloning, etc.) or chemical synthesis. Isolated nucleic acid molecules include any nucleic acid molecules and homologues or variants thereof that are part of a gene described herein and/or that encode a protein described herein, including, but not limited to, natural allelic variants and modified nucleic acid molecules (homologues or variants) in which nucleotides have been inserted, deleted, substituted, and/or inverted in such a manner that such modifications provide the desired effect on protein biological activity or on the activity of the nucleic acid molecule. Allelic variants and protein homologues or variants (e.g., proteins encoded by nucleic acid homologues or variants) have been discussed in detail above.

A nucleic acid molecule homologue or variant (i.e., encoding a homologue or variant of a protein of the present invention) can be produced using a number of methods known to those skilled in the art (see, for example, Sambrook et al.). For example, nucleic acid molecules can be modified using a variety of techniques including, but not limited to, by classic mutagenesis and recombinant DNA techniques (e.g., site-directed mutagenesis, chemical treatment, restriction enzyme cleavage, ligation of nucleic acid fragments and/or PCR amplification), or synthesis of oligonucleotide mixtures and ligation of mixture groups to “build” a mixture of nucleic acid molecules and combinations thereof. Another method for modifying a recombinant nucleic acid molecule encoding a protein is gene shuffling (i.e., molecular breeding) (See, for example, U.S. Pat. No. 5,605,793 to Stemmer; Minshull and Stemmer; 1999, Curr. Opin. Chem. Biol. 3:284-290; Stemmer, 1994, P. N. A. S. USA 91:10747-10751). This technique can be used to efficiently introduce multiple simultaneous changes in the protein. Nucleic acid molecule homologues or variants can be selected by hybridization with a gene or polynucleotide, or by screening for the function of a protein encoded by a nucleic acid molecule (i.e., biological activity).

The minimum size of a nucleic acid molecule of the present invention is a size sufficient to encode a protein (including a fragment, homologue, or variant of a full-length protein) having biological activity, sufficient to encode a protein comprising at least one epitope which binds to an antibody, or sufficient to form a probe or oligonucleotide primer that is capable of forming a stable hybrid with the complementary sequence of a nucleic acid molecule encoding a natural protein (e.g., under moderate, high, or high stringency conditions). As such, the size of the nucleic acid molecule encoding such a protein can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein encoding sequence, a nucleic acid sequence encoding a full-length protein (including a gene), including any length fragment between about 20 nucleotides and the number of nucleotides that make up the full length cDNA encoding a protein, in whole integers (e.g., 20, 21, 22, 23, 24, 25 . . . nucleotides), or multiple genes, or portions thereof.

The phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

In one aspect, the polynucleotide probes or primers of the invention are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or, ³²P) enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate such as: artificial membranes, organic supports, biopolymer supports and inorganic supports.

One aspect of the present invention relates to a recombinant nucleic acid molecule which comprises the isolated nucleic acid molecule described above which is operatively linked to at least one expression control sequence. More particularly, according to the present invention, a recombinant nucleic acid molecule typically comprises a recombinant vector and any one or more of the isolated nucleic acid molecules as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remains separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one aspect, a recombinant vector used in a recombinant nucleic acid molecule of the present invention is an expression vector. As used herein, the phrase “expression vector” is used to refer to a vector that is suitable for production of an encoded product (e.g., a protein of interest, such as an enzyme of the present invention). In this aspect, a nucleic acid sequence encoding the product to be produced (e.g., the protein or homologue or variant thereof) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

Typically, a recombinant nucleic acid molecule includes at least one nucleic acid molecule of the present invention operatively linked to one or more expression control sequences (e.g., transcription control sequences or translation control sequences). As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced. Transcription control sequences may also include any combination of one or more of any of the foregoing.

Recombinant nucleic acid molecules of the present invention can also contain additional regulatory sequences, such as translation regulatory sequences, origins of replication, and other regulatory sequences that are compatible with the recombinant cell. In one aspect, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another aspect, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

According to the present invention, the term “transfection” is generally used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants and describes an inherited change due to the acquisition of exogenous nucleic acids by the microorganism that is essentially synonymous with the term “transfection.” Transfection techniques include, but are not limited to, transformation, particle bombardment, electroporation, microinjection, lipofection, adsorption, infection and protoplast fusion.

One or more recombinant molecules of the present invention can be used to produce an encoded product (e.g., a protein) of the present invention. In one aspect, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., filamentous fungi or yeast or mushrooms), algal, plant, insect, or animal cell that can be transfected. Host cells can be either untransfected cells or cells that are already transfected with at least one other recombinant nucleic acid molecule.

Suitable cells (e.g., a host cell or production organism) may include any microorganism (e.g., a bacterium, a protist, an alga, a fungus, or other microbe), and is preferably a bacterium, a yeast or a filamentous fungus. Suitable bacterial genera include, but are not limited to, Escherichia, Bacillus, Lactobacillus, Pseudomonas and Streptomyces. Suitable bacterial species include, but are not limited to, Escherichia coli, Bacillus subtilis, Bacillus licheniformis, Bacillus Stearothermophilus, Lactobacillus brevis, Pseudomonas aeruginosa and Streptomyces lividans. Suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Hansenula polymorpha, Pichia pastoris, P. canadensis, Kluyveromyces marxianus and Phaffia rhodozyma.

Suitable fungal genera include, but are not limited to, Chrysosporium, Thielavia, Neurospora, Aureobasidium, Filibasidium, Piromyces, Corynascus, Cryptococcus, Acremonium, Tolypocladium, Scytalidium, Schizophyllum, Sporotrichum, Penicillium, Gibberella, Myceliophthora, Mucor, Aspergillus, Fusarium, Humicola, and Trichoderma, and anamorphs and teleomorphs thereof. Suitable fungal species include, but are not limited to, Aspergillus niger, Aspergillus oryzae, Aspergillus nidulans, Aspergillus japonicus, Absidia coerulea, Rhizopus oryzae, Myceliophthora thermophila, Neurospora crassa, Neurospora intermedia, Trichoderma reesei, Trichoderma longibrachiatum, Penicillium canescens, Penicillium solitum, Penicillium funiculosum, Myceliophthora thermophila, Acremonium alabamense, Thielavia terrestris, Sporotrichum thermophile, Sporotrichum cellulophilum, Chaetomium globosum, Corynascus heterothallicus, and Talaromyces flavus. In another aspect, a while (low cellulose) strain is sued. In one aspect, the host cell is a fungal cell of Strain C1 (VKM F-3500 D) or a mutant strain derived therefrom (e.g., UV13-6 (Accession No. VKM F-3632 D); NG7C-19 (Accession No. VKM F-3633 D); UV18-25 (VKM F-3631D), W1L (CBS122189), or W1L#100L (CBS122190)). The C1 strain was initially classified as Myceliophthora thermophila based on morphological characteristics and was subsequently reclassified as M. thermophila based on genetic tests. Host cells can be either untransfected cells or cells that are already transfected with at least one other recombinant nucleic acid molecule. Additional aspects of the present invention include any of the genetically modified cells described herein.

In another aspect, suitable host cells include insect cells (most particularly Drosophila melanogaster cells, Spodoptera frugiperda Sf9 and Sf21 cells and Trichoplusia High-Five cells), nematode cells (particularly C. elegans cells), avian cells, amphibian cells (particularly Xenopus laevis cells), reptilian cells, and mammalian cells (most particularly human, simian, canine, rodent, bovine, or sheep cells, e.g. NIH3T3, CHO (Chinese hamster ovary cell), COS, VERO, BHK, HEK, and other rodent or human cells).

In one aspect, one or more protein(s) expressed by an isolated nucleic acid molecule of the present invention are produced by culturing a cell that expresses the protein (i.e., a recombinant cell or recombinant host cell) under conditions effective to produce the protein. In some instances, the protein may be recovered, and in others, the cell may be harvested in whole, either of which can be used in a composition.

Microorganisms used in the present invention (including recombinant host cells or genetically modified microorganisms) are cultured in an appropriate fermentation medium. An appropriate, or effective, fermentation medium refers to any medium in which a cell of the present invention, including a genetically modified microorganism (described below), when cultured, is capable of expressing enzymes useful in the present invention and/or of catalyzing the production of sugars from lignocellulosic biomass. The microorganisms can be cultured by any fermentation process which includes, but is not limited to, batch, fed-batch, cell recycle, and continuous fermentation. In general the fungal strains are grown in fermenters, optionally centrifuged or filtered to remove biomass, and optionally concentrated, formulated, and dried to produce an enzyme(s) or a multi-enzyme composition that is a crude fermentation product. Particularly suitable conditions for culturing filamentous fungi are described, for example, in U.S. Pat. No. 6,015,707 and U.S. Pat. No. 6,573,086, supra.

Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential precipitation or solubilization.

Proteins of the present invention are preferably retrieved, obtained, and/or used in “substantially pure” form. As used herein, “substantially pure” refers to a purity that allows for the effective use of the protein in any method according to the present invention. For a protein to be useful in any of the methods described herein or in any method utilizing enzymes of the types described herein according to the present invention, it is substantially free of contaminants, other proteins and/or chemicals that might interfere or that would interfere with its use in a method disclosed by the present invention (e.g., that might interfere with enzyme activity), or that at least would be undesirable for inclusion with a protein of the present invention (including homologues and variants) when it is used in a method disclosed by the present invention (described in detail below). Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the protein of interest is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites), modification of nucleic acid molecules to correspond to the codon usage of the host cell, and deletion of sequences that destabilize transcripts.

Another aspect of the present invention relates to a genetically modified microorganism that has been transfected with one or more nucleic acid molecules of the present invention. As used herein, a genetically modified microorganism can include a genetically modified bacterium, alga, yeast, filamentous fungus, or other microbe. Such a genetically modified microorganism has a genome which is modified (i.e., mutated or changed) from its normal (i.e., wild-type or naturally occurring) form such that the desired result is achieved (i.e., increased or modified activity and/or production of at least one enzyme or a multi-enzyme composition for the conversion of lignocellulosic material to fermentable sugars). Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor Labs Press or Molecular Cloning: A Laboratory Manual, third edition (Sambrook and Russell, 2001), (jointly referred to herein as “Sambrook”). A genetically modified microorganism can include a microorganism in which nucleic acid molecules have been inserted, deleted or modified (i.e., mutated; e.g., by insertion, deletion, substitution, and/or inversion of nucleotides), in such a manner that such modifications provide the desired effect within the microorganism.

In one aspect, a genetically modified microorganism can endogenously contain and express an enzyme or a multi-enzyme composition and the genetic modification can be a genetic modification of one or more of such endogenous enzymes, whereby the modification has some effect on the amount and/or quality of enzyme mixtures produced by the organism of the microorganism (e.g., increased expression of the protein by introduction of promoters or other expression control sequences, or modification of the coding region by homologous recombination to increase the activity of the encoded protein).

In another aspect, a genetically modified microorganism can endogenously contain and express an enzyme for the catalysis of oxidation-reduction reactions, and the genetic modification can be an introduction of at least one exogenous nucleic acid sequence (e.g., a recombinant nucleic acid molecule), wherein the exogenous nucleic acid sequence encodes at least one additional enzyme useful for the catalysis of oxidation-reduction reactions and/or a protein that improves the efficiency of the target enzyme. In this aspect of the invention, the microorganism can also have at least one modification to a gene or genes comprising its endogenous enzyme(s) for the catalysis of oxidation-reduction reactions or an enzyme to aid in the conversion of lignocellulosic material.

In yet another aspect, the genetically modified microorganism does not necessarily endogenously (naturally) contain an enzyme for the catalysis of oxidation-reduction reactions, but is genetically modified to introduce at least one recombinant nucleic acid molecule encoding at least one enzyme or a multiplicity of enzymes for the catalysis of oxidation-reduction reactions. Such a microorganism can be used in a method of the invention, or as a production microorganism for crude fermentation products, partially purified recombinant enzymes, and/or purified recombinant enzymes, any of which can then be used in a method of the present invention.

Once the proteins (enzymes) are expressed in a host cell, a cell extract that contains the activity to test can be generated. For example, a lysate from the host cell is produced, and the supernatant containing the activity is harvested and/or the activity can be isolated from the lysate. In the case of cells that secrete enzymes into the culture medium, the culture medium containing them can be harvested, and/or the activity can be purified from the culture medium. The extracts/activities prepared in this way can be tested using assays known in the art.

The present invention is not limited to fungi and also contemplates genetically modified organisms such as algae, bacterial, and plants transformed with one or more nucleic acid molecules of the invention. The plants may be used for production of the enzymes, and/or as the lignocellulosic material used as a substrate in the methods of the invention. Methods to generate recombinant plants are known in the art. For instance, numerous methods for plant transformation have been developed, including biological and physical transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

The most widely utilized method for introducing an expression vector into plants is based on the natural transformation system of Agrobacterium. See, for example, Horsch et al., Science 227:1229 (1985). Descriptions of Agrobacterium vector systems and methods for Agrobacterium-mediated gene transfer are provided by numerous references, including Gruber et al., supra, Miki et al., supra, Moloney et al., Plant Cell Reports 8:238 (1989), and U.S. Pat. Nos. 4,940,838 and 5,464,763.

Another generally applicable method of plant transformation is microprojectile-mediated transformation, see e.g., Sanford et al., Part. Sci. Technol. 5:27 (1987), Sanford, J. C., Trends Biotech. 6:299 (1988), Sanford, J. C., Physiol. Plant 79:206 (1990), Klein et al., Biotechnology 10:268 (1992).

Another method for physical delivery of DNA to plants is sonication of target cells. Zhang et al., Bio/Technology 9:996 (1991). Alternatively, liposome or spheroplast fusion have been used to introduce expression vectors into plants. Deshayes et al., EMBO J., 4:2731 (1985), Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p. 53 (1990); D'Halluin et al., Plant Cell 4:1495-1505 (1992) and Spencer et al., Plant Mol. Biol. 24:51-61 (1994).

Some aspects of the present invention include genetically modified organisms comprising at least one nucleic acid molecule encoding at least one enzyme of the present invention, in which the activity of the enzyme is downregulated. The downregulation may be achieved, for example, by introduction of inhibitors (chemical or biological) of the enzyme activity, by manipulating the efficiency with which those nucleic acid molecules are transcribed, the efficiency with which the resultant transcripts are translated, and the efficiency of post-translational modifications, or by “knocking out” the endogenous copy of the gene. A “knock out” of a gene refers to a molecular biological technique by which the gene in the organism is made inoperative, so that the expression of the gene is substantially reduced or eliminated. Alternatively, in some aspects the activity of the enzyme may be upregulated. The present invention also contemplates downregulating activity of one or more enzymes while simultaneously upregulating activity of one or more enzymes to achieve the desired outcome.

Proteins of the present invention, at least one protein of the present invention, compositions comprising such protein(s) of the present invention, and multi-enzyme compositions (examples of which are described above) may be used in any method where it is desirable to hydrolyze glycosidic linkages in lignocellulosic material, or any other method wherein enzymes of the same or similar function are useful.

In one aspect, the present invention includes the use of at least one protein of the present invention, compositions comprising at least one protein of the present invention, or multi-enzyme compositions in methods for hydrolyzing lignocellulose and the generation of fermentable sugars therefrom. In one aspect, the method comprises contacting the lignocellulosic material with an effective amount of one or more proteins of the present invention, composition comprising at least one protein of the present invention, or a multi-enzyme composition, whereby at least one fermentable sugar is produced (liberated). The lignocellulosic material may be partially or completely or completely degraded to fermentable sugars. Economical levels of degradation at commercially viable costs are contemplated. plated.

Typically, the amount of enzyme or enzyme composition contacted with the lignocellulose will depend upon the amount of glucan present in the lignocellulose. In some aspects, the amount of enzyme or enzyme composition contacted with the lignocellulose may be from about 0.1 to about 200 mg enzyme or enzyme composition per gram of glucan; in other aspects, from about 3 to about 20 mg enzyme or enzyme composition per gram of glucan. The invention encompasses the use of any suitable or sufficient amount of enzyme or enzyme composition between about 0.1 mg and about 200 mg enzyme per gram glucan, in increments of 0.05 mg (i.e., 0.1 mg, 0.15 mg, 0.2 mg . . . 199.9 mg, 199.95 mg, 200 mg).

In a further aspect, the invention provides a method for degrading DDG, preferably, but not limited to, DDG derived from corn, to sugars. The method comprises contacting the DDG with a protein of the present invention, a composition comprising at least one protein of the present invention, or a multi-enzyme composition. In certain aspects, at least 10% of fermentable sugars are liberated. In other aspect, the at least 15% of the sugars are liberated, or at least 20% of the sugars are liberated, or at least 23% of the sugars are liberated, or at least 24% of the sugars are liberated, or at least 25% of the sugars are liberated, or at least 26% of the sugars are liberated, or at least 27% of the sugars are liberated, or at least 28% of the sugars are liberated.

In another aspect, the invention provides a method for producing fermentable sugars comprising cultivating a genetically modified microorganism of the present invention in a nutrient medium comprising a lignocellulosic material, whereby fermentable sugars are produced.

Also provided are methods that comprise further contacting the lignocellulosic material with at least one accessory enzyme. Accessory enzymes have been described elsewhere herein. The accessory enzyme or enzymes may be added at the same time, prior to, or following the addition of a protein of the present invention, a composition comprising at least one protein of the present invention, or a multi-enzyme composition, or can be expressed (endogenously or overexpressed) in a genetically modified microorganism used in a method of the invention. When added simultaneously, the protein of the present invention, a composition comprising at least one protein of the present invention, or a multi-enzyme composition will be compatible with the accessory enzymes selected. When the enzymes are added following the treatment with the protein of the present invention, a composition comprising at least one protein of the present invention, or a multi-enzyme composition, the conditions (such as temperature and pH) may be altered to those optimal for the accessory enzyme before, during, or after addition of the accessory enzyme. Multiple rounds of enzyme addition are also encompassed. The accessory enzyme may also be present in the lignocellulosic material itself as a result of genetically modifying the plant. The nutrient medium used in a fermentation can also comprise one or more accessory enzymes.

In some aspects, the method comprises a pretreatment process. In general, a pretreatment process will result in components of the lignocellulose being more accessible for downstream applications or so that it is more digestible by enzymes following treatment in the absence of hydrolysis. The pretreatment can be a chemical, physical or biological pretreatment. The lignocellulose may have been previously treated to release some or all of the sugars, as in the case of DDG. Physical treatments, such as grinding, boiling, freezing, milling, vacuum infiltration, and the like may also be used with the methods of the invention. In one aspect, the heat treatment comprises heating the lignocellulosic material to 121° C. for 15 minutes. A physical treatment such as milling can allow a higher concentration of lignocellulose to be used in the methods of the invention. A higher concentration refers to about 20%, up to about 25%, up to about 30%, up to about 35%, up to about 40%, up to about 45%, or up to about 50% lignocellulose. The lignocellulose may also be contacted with a metal ion, ultraviolet light, ozone, and the like. Additional pretreatment processes are known to those skilled in the art, and can include, for example, organosolv treatment, steam explosion treatment, lime impregnation with steam explosion treatment, hydrogen peroxide treatment, hydrogen peroxide/ozone (peroxone) treatment, acid treatment, dilute acid treatment, and base treatment, including ammonia fiber explosion (AFEX) technology. Details on pretreatment technologies and processes can be found in Wyman et al., Bioresource Tech. 96:1959 (2005); Wyman et al., Bioresource Tech. 96:2026 (2005); Hsu, “Pretreatment of biomass” In Handbook on Bioethanol: Production and Utilization, Wyman, Taylor and Francis Eds., p. 179-212 (1996); and Mosier et al., Bioresource Tech. 96:673 (2005).

In some aspects, the methods may be performed one or more times in whole or in part. That is, one may perform one or more pretreatments, followed by one or more reactions with a protein of the present invention, composition or product of the present invention and/or accessory enzyme. The enzymes may be added in a single dose, or may be added in a series of small doses. Further, the entire process may be repeated one or more times as necessary. Therefore, one or more additional treatments with heat and enzymes are contemplated.

The methods described above result in the production of fermentable sugars. During, or subsequent to the methods described, the fermentable sugars may be recovered and/or purified by any method known in the art. The sugars can be subjected to further processing; e.g., they can also be sterilized, for example, by filtration.

In an additional aspect, the invention provides a method for producing an organic substance, comprising saccharifying a lignocellulosic material with an effective amount of a protein of the present invention or a composition comprising at least one protein of the present invention, fermenting the saccharified lignocellulosic material obtained with one or more fermentating microorganisms, and recovering the organic substance from the fermentation. Sugars released from biomass can be converted to useful fermentation products including but not limited to amino acids, vitamins, pharmaceuticals, animal feed supplements, specialty chemicals, chemical feedstocks, plastics, solvents, fuels, or other organic polymers, lactic acid, and ethanol, including fuel ethanol. Specific products that may be produced by the methods of the invention include, but not limited to, biofuels (including ethanol); lactic acid; plastics; specialty chemicals; organic acids, including citric acid, succinic acid, itaconic and maleic acid; solvents; animal feed supplements; pharmaceuticals; vitamins; amino acids, such as lysine, methionine, tryptophan, threonine, and aspartic acid; industrial enzymes, such as proteases, cellulases, amylases, glucanases, lactases, lipases, lyases, oxidoreductases, and transferases; and chemical feedstocks. The methods of the invention are also useful to generate feedstocks for fermentation by fermenting microorganisms. In one aspect, the method further comprises the addition of at least one fermenting organism.

As used herein, “fermenting organism” refers to an organism capable of fermentation, such as bacteria and fungi, including yeast. Such feedstocks have additional nutritive value above the nutritive value provided by the liberated sugars.

In some aspects the invention comprises, but is not limited to methods for oxidoreductases in the biofuel industry, such as lignin degradation.

In some aspects the invention comprises, but is not limited to additional methods for oxidoreductases, such as biosensors; diagnostic (analytical) kits; effective additives for refolding immunoglobulin-folded proteins in vitro; bleaching cotton; polymerizing phenols and aromatic amines; asymmetric syntheses of amino acids, steroids, pharmaceuticals and other fine chemicals; biocatalysis; pollution control, and oxygenation of hydrocarbons; treatment of industrial waste waters (detoxification); soil detoxification; manufacturing of adhesives, computer chips, car parts, and linings of drums and cans; whitening the skin/hair/teeth; and stimulating the immune system.

Exemplary methods according to the invention are presented below. Examples of the methods described above may also be found in the following references: Trichoderma & Gliocladium, Volume 2, Enzymes, biological control and commercial applications, Editors: Gary E. Harman, Christian P. Kubicek, Taylor & Francis Ltd. 1998, 393 (in particular, chapters 14, 15 and 16); Helmut Uhlig, Industrial enzymes and their applications, Translated and updated by Elfriede M. Linsmaier-Bednar, John Wiley & Sons, Inc 1998, p. 454 (in particular, chapters 5.1, 5.2, 5.3, 5.4, 5.5, 5.6, 5.7, 5.9, 5.10, 5.11, and 5.13). For saccharification applications: Hahn-Hägerdal, B., Galbe, M., Gorwa-Grauslund, M. F. Lidén, Zacchi, G. Bio-ethanol—the fuel of tomorrow from the residues of today, Trends in Biotechnology, 2006, 24 (12), 549-556; Mielenz, J. R. Ethanol production from biomass: technology and commercialization status, Current Opinion in Microbiology, 2001, 4, 324-329; Himmel, M. E., Ruth, M. F., Wyman, C. E., Cellulase for commodity products from cellulosic biomass, Current Opinion in Biotechnology, 1999, 10, 358-364; Sheehan, J., Himmel, M. Enzymes, energy, and the environment: a strategic perspective on the U.S. Department of Energy's Research and Development Activities for Bioethanol, Biotechnology Progress. 1999, 15, 817-827. For textile processing applications: Galante, Y. M., Formantici, C, Enzyme applications in detergency and in manufacturing industries, Current Organic Chemistry, 2003, 7, 1399-1422. For pulp and paper applications: Bajpai, P., Bajpai, P. K Deinking with enzymes: a review. TAPPI Journal, 1998, 81(12), 111-117; Viikari, L., Pere, J., Suurnäkki, A., Oksanen, T., Buchert, J. Use of cellulases in pulp and paper applications. In: “Carbohydrates from Trichoderma reesei and other microorganisms. Structure, Biochemistry, Genetics and Applications.” Editors: Mark Claessens, Wim Nerinckx, and Kathleen Piens, The Royal Society of Chemistry 1998, 245-254. For food and beverage applications: Roller, S., Dea, I. C. M. Biotechnology in the production and modification of biopolymers for foods, Critical Reviews in Biotechnology, 1992, 12(3), 261-277.

Additional references include, U.S. Pat. No. 5,529,926; U.S. Pat. No. 6,746,679; U.S. Pat. No. 7,732,178; U.S. Pat. No. 6,660,128; U.S. Pat. No. 6,093,436; U.S. Pat. No. 5,691,193; U.S. Pat. No. 5,785,811; U.S. Pat. No. 7,329,424.

Additional assays and methods for examining the activity of the enzymes are found in U.S. patent application Ser. Nos. 60/806,876, 60/970,876, 11/487,547, 11/775,777, 11/833,133, and 12/205,694 and incorporated herein by reference.

Additional assays and methods for examining the activity of the enzymes are found in U.S. patent application Ser. Nos. 60/806,876, 60/970,876, 11/487,547, 11/775,777, 11/833,133, and 12/205,694 and incorporated herein by reference.

Using the examples set out below, fungal strains can be generated which lack functional genes encoding enzymes causing the formation of cellobionolactone, cellobionic acid, gluconolactone, or gluconic acid by any of a variety of genetic methods, such as gene deletion, gene disruption, or mutation. The following examples are provided for the purpose of illustration and are not intended to limit the scope of the present invention.

### Example 1

### Inhibition of Cellulase Activity by Gluconolactone/Gluconic Acid

Purified Bgl1, Eg5, Eg6, CBH1, CBH2, and CBH4 from M. thermophila C1 were used to determine the level of inhibition by gluconolactone. For Bgl1, cellobiase activity was assayed by the following procedure: 0.4 ml of 2.5 mM cellobiose solution (in 0.1 M Na-acetate with pH 4.5, 5.0 or 6.0) was incubated during 5 min at 50° C. (with or without gluconolactone), then 0.1 ml of the enzyme (bgl1 from C1) solution was added (the dilution of the enzyme chosen at such a concentration in order to achieve 10% of cellobiose hydrolysis in 15 min, which is 0.072 g/L of glucose released). After 5, 10 and 15 min of incubation at 40° C., 0.1 ml of the reaction mixture was sampled and glucose concentration was determined immediately by the glucose oxidase-peroxidase assay (Megazymes). Gluconolactone (Sigma-Aldrich) was added in the reaction mixture at concentrations between 0-10 g/L (0, 0.04, 0.08, 0.1, 0.2, 0.3, 0.6, 0.8, 1, 2, 4, 5, 8 and 10 g/L).

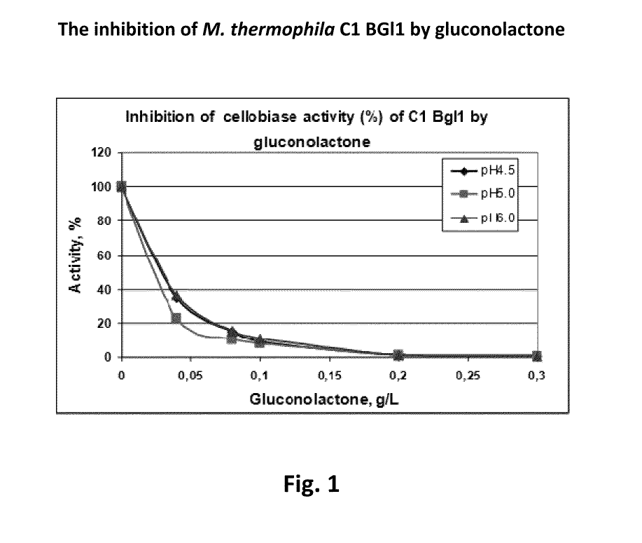

FIG. 1 shows the inhibition of M. thermophila C1 BGl1 by gluconolactone. The results clearly show that gluconolactone has a negative effect on the cellobiase activity. The enzyme was completely inhibited by gluconolactone at the concentrations of gluconolactone above 0.1 g/L at pH 4.5, 5.0 and 6.0. For C1 cellulases Eg2, Eg5, Eg6, CBH1, CBH2, CBH4, the effect of 10 g/L gluconolactone on the saccharification of microcrystalline cellulose (“MCC”) at 50° C. was determined after 72 hrs. Table 1 shows the level of inhibition for the purified cellulases Eg2, Eg5, Eg6, CBH1, CBH2, and CBH4.

TABLE 1
Inhibition by 10 g/L of gluconolactone

| C1 Enzyme | Remaining activity |
|---|---|
| EG2 | 28% |
| EG5 | 65% |
| EG6 | 7 |
| CBH1 | 14% |
| CBH2 | 13% |
| CBH4 | 33% |

### Example 2

### Construction of a Myceliophthora thermophila Strain Containing a CDH1 Gene Disruption

A derivative of the M. thermophila C1 strain UV18-25 (Accession No. VKM F-3631 D) was selected as the target strain for the cdh1 gene disruption. In order to create a cdh1 gene disruption strain, part of the cdh1 gene was deleted by replacing it with an AmdS selection marker. In short, the upstream region of the cdh1 gene was amplified using primers

(SEQ ID NO: 1)
5′-CACAAGCACTGCGAGTACCAC-3′
and
(SEQ ID NO: 2)
5′-GTCGAGCTTCATTTTTTCGAAGCGCAGCAACTTCAAG-3′;

and an internal region of the cdh1 gene was amplified using primers

(SEQ ID NO: 3)
5′-CTTGAAGTTGCTGCGCTTCGAACTACCTAGTTTGTGTGTG-3′
and
(SEQ ID NO: 4)
5′-CACCGTTCTCCGCTTCTCAC-3′.

These two PCR products were then fused in a fusion PCR experiment using primers

(SEQ ID NO: 1)
5′-CACAAGCACTGCGAGTACCAC-3′
and
(SEQ ID NO: 4)
5′-CACCGTTCTCCGCTTCTCAC -3′.

The resulting PCR product was subsequently cloned into the pGEMTeasy vector (Promega) and into this vector a DNA fragment was cloned containing the AmdS marker (SEQ ID NO: 5) using BstBt1. The resulting product was then digested with Not1 to obtain a DNA fragment containing the amdS marker flanked by the cdh1 regions. The fragment was used to transform the C1 strain. Purification streaks of hundreds of transformants were made on acetamide plates and incubated for 4 days at 35° C. Pure colonies were transferred to microtiterplate (MTP) wells containing Caylase medium, grown and transferred to MTP wells containing production medium and grown. Supernatants from MTPs were assayed for cellobiose dehydrogenase activity, based on the reduction of ferricyanide to ferrocyanide by CDH. Gene-disruption candidates were analyzed to identify transformants that have a disruption of the cdh1 gene by PCR. Southern blotting was used to verify the correct disruption of cdh1.

### Example 3

### Construction of a M. thermophila Strain Containing a Cdh1 Gene Disruption and a Cdh2 Gene Disruption

The AmdS selection marker was removed from the strain derived from M. thermophila C1 strain UV18-25 (Accession No. VKM F-3631 D) containing the cdh1 gene disruption (described in example 2) by methodologies well known in the art, that encompassed counterselection on fluoro-acetamide plates, and Southern analysis of positive candidates to verify to the correct removal of the AmdS marker. The resulting strain was used as the target strain for a cdh2 gene disruption.

In short, the upstream region of the cdh2 gene was amplified using primers

(SEQ ID NO: 6)
5′-CAACACGAGACCCGAGATGG-3′
and
(SEQ ID NO: 7)
5′-CATTGGTTGGTACGTGAGGGTTCGAACCATAAGAGCGGAGGTC
AGG-3′;

and the downstream region of the cdh2 gene was amplified using primers

(SEQ ID NO: 8)
5′-CCTGACCTCCGCTCTTATGGTTCGAATTAGAGGTCTTGTTGGG
CCT-7′
and
(SEQ ID NO: 9)
5′-GAGCGGCTTTGGCAATTGAG-3′.

The upstream fragment was cloned into the pGEMTeasy vector (Promega) and subsequently excised using BstB1 and PstI. The downstream fragment was also cloned into the pGEMTeasy vector. This vector containing the downstream fragment was subsequently digested using BstB1 and pstI and this was ligated to the upstream fragment was then ligated. Into the resulting vector the DNA fragment was cloned containing the amdS marker (SEQ ID NO: 5) using BstBt1. The resulting product was then digested with NotI to obtain a DNA fragment containing the amdS marker flanked by the cdh2 regions. The fragment was used to transform the M. thermophila C1 strain containing the cdh1 disruption. Purification streaks of hundreds of transformants were made on acetamide plates and incubated for 4 days at 35° C. Pure colonies were transferred to microtiterplate (MTP) wells containing Caylase medium, grown and transferred to MTP wells containing complete medium and grown for 48 hours. gDNA was isolated from all transformants and PCR was used to identify transformants with a correct cdh2 disruption. Southern blotting was used to verify to correct disruption of both cdh1 and cdh2.

### Example 4

### Production of Enzyme Mixtures by Modified Fungal Strains

Methods of producing enzyme mixtures from modified fungal strains are disclosed in U.S. Pat. No. 7,923,236, specifically incorporated by reference, herein. A brief summary of these methods are described below. Modified fungal cells of the present invention are cultured in an appropriate fermentation medium. An appropriate, or effective, fermentation medium refers to any medium in which a cell of the present invention, including a modified organism (described below), when cultured, is capable of expressing enzymes useful in the present invention and/or of catalyzing the production of sugars from lignocellulosic biomass. Such a medium is typically an aqueous medium comprising assimilable carbon, nitrogen and phosphate sources, which can also include appropriate salts, minerals, metals and other nutrients. Microorganisms and other cells of the present invention can be cultured in conventional fermentation bioreactors. The microorganisms can be cultured by any fermentation process which includes, but is not limited to, batch, fed-batch, cell recycle, and continuous fermentation. The fermentation of microorganisms such as fungi may be carried out in any appropriate bioreactor, using methods known to those skilled in the art. For example, the fermentation may be carried out for a period of 1 to 14 days, or more preferably between about 3 and 10 days. The temperature of the medium is typically maintained between about 25° C. and 50° C., and more preferably between 28° C. and 40° C. The pH of the fermentation medium is regulated to a pH suitable for growth and protein production of the particular organism. The bioreactor can be aerated in order to supply the oxygen necessary for fermentation and to avoid the excessive accumulation of carbon dioxide produced by fermentation. In addition, the aeration helps to control the temperature and the moisture of the culture medium. In general, the fungal strains are grown in bioreactors, optionally centrifuged or filtered to remove biomass, and optionally concentrated, formulated, and dried to produce an enzyme(s) or a multi-enzyme composition that is a crude fermentation product. Particularly suitable conditions for culturing filamentous fungi are described, for example, in U.S. Pat. Nos. 6,015,707 and 6,573,086, supra.

### Example 5

### Analysis of Enzyme Compositions Produced by Myceliophthora thermophila C1 Strains

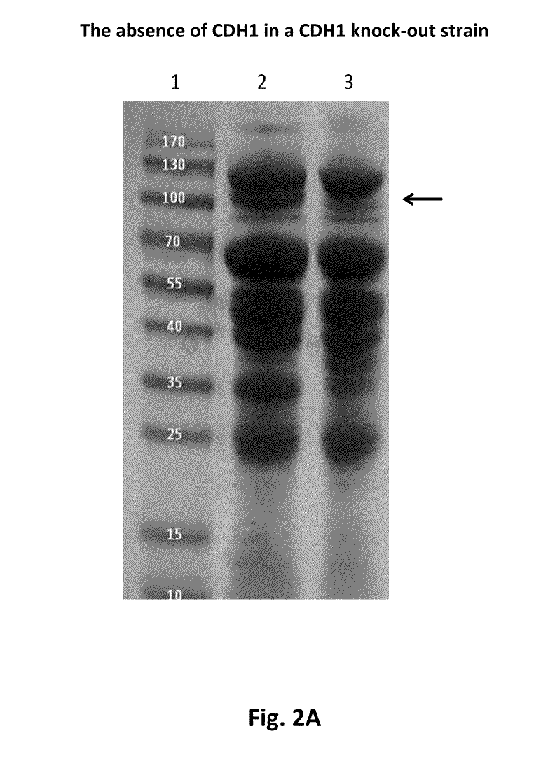

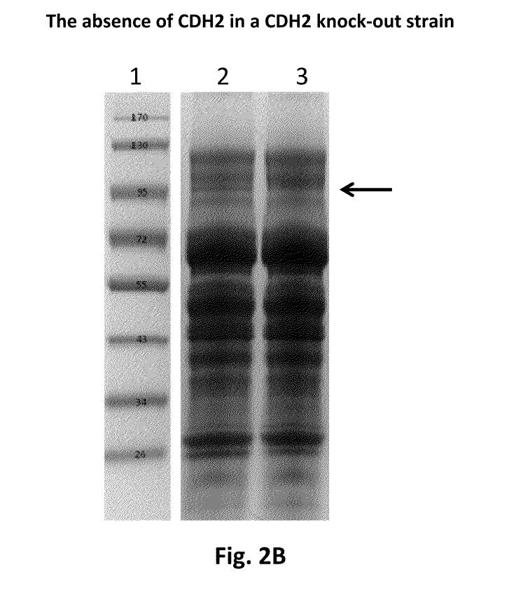

The M. thermophila C1 strain, containing the cdh1-gene disruption (described in example 2), the M. thermophila C1 strain containing both the cdh1 gene disruption and the cdh2 gene disruption (described in example 3) and the M. thermophila C1 ancestor strain were fermented for cellulase production. SDS-PAGE was used to analyze the produced enzyme mixtures. FIGS. 2A and B show the SDS-PAGE of the enzyme mixtures produced. Compared to the enzyme mixture produced by the ancestor strain, the mixture produced by the strain containing the cdh1-gene disruption clearly showed the absence of CDH1 (FIG. 2A, lane 1: protein standard; 2: ancestor and 3 cdh1-gene disruption). The mixture produced by the strain also containing the cdh2 gene disruption clearly showed, in addition, the absence of CDH2 (FIG. 2B, lane 1: protein standard; 2: cdh1-gene disruption and 3: cdh1/cdh2-gene disruption).

### Example 6

### Cellobiose Dehydrogenase Activity Assay

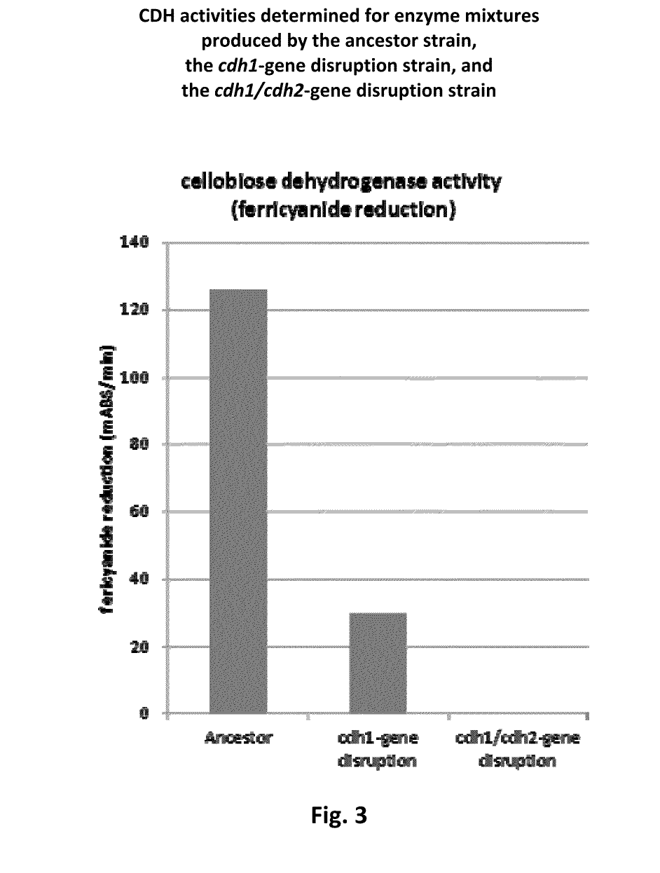

Cellobiose dehydrogenase activity was determined by measuring the reduction of 0.375 mM of ferricyanide at 420 nm using 2.5 mM of cellobiose as the substrate. The assay was carried out in a total volume of 1 mL at 35° C. in 25 mM NaAc pH 4.5. FIG. 3 shows the CDH activity as determined for enzyme mixtures produced by the ancestor strain, by the cdh1-gene disruption strain and by the cdh1/cdh2-gene disruption strain (described in example 4). The data show that the CDH activity is greatly reduced in the enzyme mixture produced by both the cdh1-gene disruption strain as well as in the mixture produced by the cdh1/cdh2-gene disruption strain.

### Example 7

### Saccharification of Pretreated Corn Stover

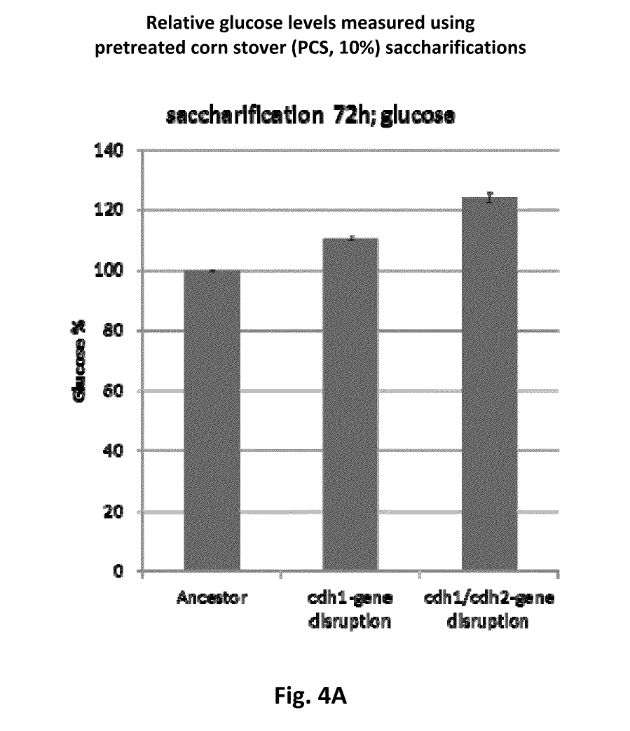

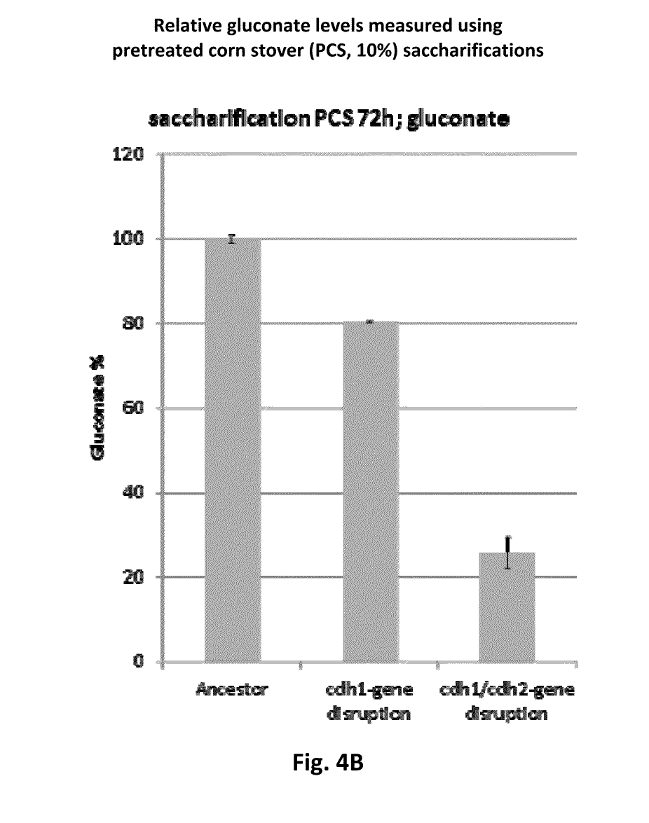

Saccharification of pretreated corn stover (PCS, 10%) by enzyme mixtures produced by the ancestor strain, by the cdh1-gene disruption strain and by the cdh1/cdh2-gene disruption strain (described in Example 4) were carried out for 72 hours at 55° C. Saccharification reactions were carried out in a total volume of 20 mL in 50 mL polypropylene tubes. The pH was adjusted to pH 5.0 using a 2 M NaOH solution in a final concentration of 100 mM. Substrate/NaOH mixtures were left for 2 hours at 55° C. and 300 rpm for the pH to stabilize before addition of the enzyme. After 24 h and 72 h, 0.2 mL samples were taken and filtrated in a micro plate (pvdf), supernatant was stored at −20° C. until further analysis. Glucose concentrations were measured using GOPOD assay (Megazymes) and the gluconic acid concentration was determined using the D-gluconic acid/D-glucono-δ-lactone assay (Megazymes). All experiments have been performed in duplicate. FIGS. 4A and 4B show the results for the PCS saccharifications. The data clearly show an increased glucose release and a decreased gluconic acid release for the enzyme mixture produced by the cdh1-gene disruption strain as well as for the mixture produced by the cdh1/cdh2-gene disruption strain.

The increase in glucose release is larger than the decrease in gluconic acid release. For the enzyme mixture produced by the cdh1-gene disruption strain the increase in glucose was 5.7 times the decrease in gluconic acid. For the cdh1/cdh2-gene disruption strain increase in glucose was 3.4 times the decrease in gluconic acid. The enzyme mixtures produced by the disruption strains also showed a reduced level of acidification during the saccharification (Table 2).

TABLE 2
Level of acidification during PCS saccharification.

| Relative | Enzyme mixture producing | Final pH after 72 hours of |
|---|---|---|
| Enzyme dosage | strain | saccharification |
| 1x | Ancestor | 4.70 |
| 1x | cdh1-gene disruption | 4.75 |
| 1x | cdh1/cdh2-gene disruption | 4.90 |
| 2x | Ancestor | 4.61 |
| 2x | cdh1-gene disruption | 4.63 |
| 2x | cdh1/cdh2-gene disruption | 4.87 |
| 4x | Ancestor | 4.53 |
| 4x | cdh1-gene disruption | 4.57 |
| 4x | cdh1/cdh2-gene disruption | 4.85 |

While the preferred aspects of the invention have been illustrated and described in detail, it will be appreciated by those skilled in the art that that various changes can be made therein without departing from the spirit and scope of the invention. Accordingly, the particular arrangements disclosed are meant to be illustrative only and not limiting as to the scope of the invention, which is to be given the full breadth of the appended claims and any equivalent thereof, as set forth in the following exemplary claims.

What is claimed is:

1. A modified fungus comprising one or more genes encoding enzymes having one or more cellulase or hemicellulase activities;
wherein said fungus comprises one or more modified genes encoding enzymes responsible for the production of one or more products selected from cellobionolactone, cellobionic acid, gluconolactone, and gluconic acid;
wherein the level of expression of said modified genes is reduced or eliminated or the level of activity of modified enzymes encoded by said modified genes is reduced or eliminated compared to the endogenous level of expression or activity in a parent fungus lacking one or more of said modifications.

2. The modified fungus of claim 1, wherein said fungus is a filamentous fungus from a genus or genus and species selected from the group consisting of Chrysosporium, Thielavia, Talaromyces, Thermomyces, Thermoascus, Neurospora, Aureobasidium, Filibasidium, Piromyces, Corynascus, Cryplococcus, Acremonium, Tolypocladium, Scytalidium, Schizophyllum, Sporotrichum, Penicillium, Gibberella, Myceliophthora, Mucor, Aspergillus, Fusarium, Humicola, and Trichoderma, and Talaromyces emersonii, plus anamorphs and teleomorphs, and derivatives thereof.

3. The modified fungus of claim 2, wherein said filamentous fungus is Myceliophthora thermophila.

4. The modified fungus of claim 3, wherein said filamentous fungus is Myceliophthora thermophila C1.

5. The modified fungus of claim 4, wherein said filamentous fungus is Garg 27K (Accession No. VKM-F-3500 D); UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190).

6. The modified fungus of claim 5, wherein the filamentous fungus is UV18-25 (Accession No. VKM F-3631 D).

7. The modified fungus of claim 4, comprising one or more modified genes encoding a cellobiose dehydrogenase.

8. The modified fungus of claim 7, wherein said modified gene is a modified cdh gene.

9. The modified fungus of claim 8, wherein the modified cdh gene is a cdh1 or a cdh2 gene.

10. The modified fungus of claim 9, wherein the cdh1 gene was removed or disrupted by removing or replacing all or part of the cdh1 gene.

11. The modified fungus of claim 10, wherein the cdh1 gene was disrupted by replacing a part of the cdh1 gene.

12. The modified fungus of claim 11, wherein the cdh1 gene was disrupted by replacing a part of the cdh1 gene with a gene encoding a selectable marker.

13. The modified fungus of claim 9, comprising a modified cdh2 gene.

14. The modified fungus of claim 9, comprising a modified cdh1 and a modified cdh2 gene.

15. The modified fungus of claim 14, wherein said filamentous fungus is Garg 27K (Accession No. VKM F-3500 D); UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190).

16. The modified fungus of claim 15, wherein the filamentous fungus is UV18-25 (Accession No. VKM F-3631 D).

17. The modified fungus of claim 1, wherein the level of expression of said modified genes, or the level of activity of modified enzymes encoded by said modified genes, is reduced or eliminated by modifying the coding sequence of one or more genes encoding said enzymes.

18. The modified fungus of claim 1, wherein the level of expression of said modified genes or the level of activity of modified enzymes is reduced or eliminated by modifying the noncoding sequence of one or more genes encoding said enzymes.

19. The modified fungus of claim 1, wherein the level of expression of said modified genes or the level of activity of modified enzymes is reduced or eliminated by introduction of one or more point insertions or deletions into the non-coding sequence of one or more genes encoding said enzymes.

20. The modified fungus of claim 1, wherein the level of expression of said modified genes or the level of activity of modified enzymes is reduced or eliminated by introduction of one or more point mutations, insertions, or deletions into the coding sequence of one or more genes encoding said enzymes.

21. The modified fungus of claim 1, wherein the level of expression of said modified genes or the level of activity of modified enzymes is reduced from about 50% to about 100% compared to the endogenous level of expression or activity in a parent fungus lacking one or more of said modified genes.

22. The modified fungus of claim 21, wherein the level of expression of said modified genes or the level of activity of modified enzymes is reduced from at least 75% compared to the endogenous level of expression or activity in a parent fungus lacking one or more of said modified genes.

23. The modified fungus of claim 22, wherein the level of expression of said modified genes or the level of activity of modified enzymes is reduced from at least 90% compared to the endogenous level of expression or activity in a parent fungus lacking one or more of said modified genes.

24. The modified fungus of claim 21, wherein the level of activity of an enzyme causing the formation of cellobionolactone or cellobionic acid is reduced from about 50% to about 100%.

25. The modified fungus of claim 24, wherein the level of activity of an enzyme causing the formation of cellobionolactone or cellobionic acid is reduced at least 75%.

26. The modified fungus of claim 25, wherein level of activity of an enzyme causing the formation of cellobionolactone or cellobionic acid is reduced at least 90%.

27. The modified fungus of claim 21, wherein level of activity of an enzyme causing the formation of gluconolactone or gluconic acid is reduced from about 50% to about 100%.

28. The modified fungus of claim 27, wherein the level of activity of an enzyme causing the formation of gluconolactone or gluconic acid is reduced at least 75%.

29. The modified fungus of claim 28, wherein level of activity of an enzyme causing the formation of gluconolactone or gluconic acid is reduced at least 90%.

30. The modified fungus of claim 1, wherein one or more genes encoding enzymes responsible for the production of one or more products selected from cellobionolactone, cellobionic acid, gluconolactone, and gluconic acid encode an enzyme selected from the group consisting of cellobiose dehydrogenase (CDH), glucooligosaccharide dehydrogenase, glucose dehydrogenase, glucooligosaccharide oxidase, cellobiose oxidase, glucose oxidase, and copper-dependent polysaccharide monooxygenase.

31. The modified fungus of claim 30, wherein the level of expression or level of activity of a polypeptide encoded by one or more genes encoding a beta-glucosidase is present at higher levels than the unmodified parent fungus.

32. The modified fungus of claim 30, wherein the level of expression or level of activity of a polypeptide encoded by one or more genes encoding a xylanase is present at higher levels than the unmodified parent fungus.

33. The modified fungus of claim 30, wherein the level of expression or level of activity of a polypeptide encoded by one or more genes encoding a copper-dependent polysaccharide monooxygenase is present at higher levels than the unmodified parent fungus.

34. The modified fungus of claim 1, wherein one or more genes encoding enzymes responsible for the production of one or more products selected from cellobionolactone, cellobionic acid, gluconolactone, and gluconic acid encode a cellobiose dehydrogenase (CDH).

35. The modified fungus of claim 34, wherein the amino acid sequence of the cellobiose dehydrogenase (CDH) is selected from a group of polypeptides having at least 90% homology with any of the polypeptides of SEQ ID NOS: 10-12.

36. The modified fungus of claim 34, wherein the amino acid sequence of the cellobiose dehydrogenase (CDH) is selected from a group of polypeptides having at least 95% homology with any of the polypeptides of SEQ ID NOS: 10-12.

37. The modified fungus of claim 34, wherein the amino acid sequence of the cellobiose dehydrogenase (CDH) is selected from a group of polypeptides having at least 99% homology with any of the polypeptides of SEQ ID NO: 10-12.

38. The modified fungus of claim 34, wherein the cellobiose dehydrogenase (CDH) is CDH1 (SEQ ID NO: 10).

39. The modified fungus of claim 34, wherein the cellobiose dehydrogenase (CDH) is CDH2 (SEQ ID NO: 11).

40. The modified fungus of claim 34, wherein the cellobiose dehydrogenase (CDH) is CDH3 (SEQ ID NO: 12).

41. The modified fungus of claim 34, wherein CDH activity is reduced from about 50% to about 100% when measured by a ferricyanide reduction assay.

42. The modified fungus of claim 41, wherein CDH activity is reduced at least 75% when measured by a ferricyanide reduction assay.

43. The modified fungus of claim 41, wherein CDH activity is reduced at least 90% when measured by a ferricyanide reduction assay.

44. The modified fungus of claim 41, wherein CDH activity is reduced at least 95% when measured by a ferricyanide reduction assay.

45. The modified fungus claim 34, wherein the level of expression of at least one modified gene encoding a cellobiose dehydrogenase or the level of activity of at least one cellobiose dehydrogenase is reduced or eliminated.

46. The modified fungus of claim 34, wherein the level of expression of at least two modified genes encoding cellobiose dehydrogenases or the level of activity of at least two cellobiose dehydrogenases are reduced or eliminated.

47. The modified fungus of claim 1, wherein one or more of the modified genes encode an enzyme selected from the group consisting of glucooligosaccharide dehydrogenase, glucooligosaccharide oxidase, and copper-dependent polysaccharide monooxygenase.

48. The modified fungus of claim 47, wherein one or more of said modified genes encode a glucooligosaccharide oxidase.

49. The modified fungus of claim 47, wherein the amino acid sequence of the glucooligosaccharide oxidase is selected from a group of polypeptides having at least 90% homology with any of the polypeptides of SEQ ID NOS: 13-14.

50. The modified fungus of claim 47, wherein the amino acid sequence of the glucooligosaccharide oxidase is selected from a group of polypeptides having at least 95% homology with any of the polypeptides of SEQ ID NOS: 13-14.

51. The modified fungus of claim 47, wherein the amino acid sequence of the glucooligosaccharide oxidase is selected from a group of polypeptides having at least 99% homology with any of the polypeptides of SEQ ID NOS: 13-14.

52. The modified fungus of claim 1, wherein one or more of the modified genes encode a copper-dependent polysaccharide monooxygenase.

53. The modified fungus of claim 52, wherein the amino acid sequence of copper-dependent polysaccharide monooxygenase is selected from a group of polypeptides having at least 90% homology with any of the polypeptides of SEQ ID NOS: 15-41.

54. The modified fungus of claim 52, wherein the amino acid sequence of copper-dependent polysaccharide monooxygenase is selected from a group of polypeptides having at least 95% homology with any of the polypeptides of SEQ ID NOS: 15-41.

55. The modified fungus of claim 52, wherein the amino acid sequence of the copper-dependent polysaccharide monooxygenase is selected from a group of polypeptides having at least 99% homology with any of the polypeptides of SEQ ID NOS: 15-41.

56. The modified fungus of claim 1, wherein one or more of the modified genes encode an oxidase.

57. The modified fungus of claim 56, wherein the amino acid sequence of the oxidase is selected from a group of polypeptides having at least 90% homology with any of the polypeptides of SEQ ID NOS: 42-52.

58. The modified fungus of claim 56, wherein the amino acid sequence of the oxidase is selected from a group of polypeptides having at least 95% homology with any of the polypeptides of SEQ ID NOS: 42-52.

59. The modified fungus of claim 56, wherein the amino acid sequence of the oxidase is selected from a group of polypeptides having at least 99% homology with any of the polypeptides of SEQ ID NOS: 42-52.

60. The modified fungus of claim 1, further comprising a modified gene encoding a protease wherein the level of expression said modified gene or level of activity of said modified protease is present at lower levels than the unmodified parent fungus.

61. A composition for the degradation and saccharification of (ligno)cellulosic materials comprising a mixture of enzymes obtained from a modified fungus,
wherein said composition has one or more enzymes having cellulase or hemicellulase activities, and lacks or has reduced levels or activities of one or more enzymes responsible for the production of one or more products selected from the group consisting of cellobionolactone, cellobionic acid, gluconolactone, and gluconic acid;
wherein production of glucose with said composition in the presence of (ligno)cellulosic materials is enhanced above the endogenous level of glucose produced with a composition which has normal levels or activities of one or more enzymes responsible for the production of one or more products selected from cellobionolactone, cellobionic acid, gluconolactone, and gluconic acid.

62. The composition of claim 61, wherein the cellulase is selected from the group consisting of cellobiohydrolase, beta-glucosidase, and endoglucanase.

63. The composition of claim 61, wherein the hemicellulase is selected from at least one beta-xylosidase, a xylanase, an arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xyloglucanase, and a beta glucosidase.

64. The composition of claim 61, wherein the reduced levels or activities of enzymes responsible for the production of one or more products selected from cellobionolactone, cellobionic acid, gluconolactone, gluconic acid is due to the downregulation, deletion, or mutation of at least one enzyme selected from cellobiose dehydrogenase, glucooligosaccharide dehydrogenase, glucose dehydrogenase, glucooligosaccharide oxidase, cellobiose oxidase, glucose oxidase, and copper-dependent polysaccharide monooxygenase.

65. The composition of claim 61, wherein at least one of the enzymes of SEQ ID NOS: 10-52 is absent.

66. The composition of claim 61, wherein the level or activity of one or more enzymes responsible for the production of one or more products selected from cellobionolactone, cellobionic acid, gluconolactone, gluconic acid
is eliminated or reduced in the presence of an inhibiting amount of at least one inhibitor of said enzymes.

67. The composition of claim 61, wherein the level or activity of one or more enzymes responsible for the production of one or more products selected from cellobionolactone, cellobionic acid, gluconolactone, gluconic acid
is eliminated or reduced by total or partial inactivation of at least one of said enzymes.

68. The composition of claim 67, wherein at wherein the level or activity of one or more enzymes responsible for the production of one or more products selected from cellobionolactone, cellobionic acid, gluconolactone, gluconic acid
is eliminated or reduced by total or partial inactivation of at least two of said enzymes.

69. The composition of claim 61, wherein the level or activity of one or more enzymes responsible for the production of one or more products selected from cellobionolactone, cellobionic acid, gluconolactone, gluconic acid
is eliminated or reduced by removal of at least one of said enzymes.

70. The composition of claim 61, wherein at least one of said enzymes is obtained from a modified fungus modified by random mutagenesis.

71. The composition of claim 61, wherein at least one of said enzymes is obtained from a modified fungus modified by directed mutagenesis.

72. A method of increasing saccharification of cellulosic materials comprising:
treating the cellulosic material with an enzyme composition comprising enzymes having one or more cellulase or hemicellulase activities;
wherein the enzyme composition is obtained from a modified fungus comprising one or more modified genes encoding enzymes responsible for the production of one or more products selected from cellobionolactone, cellobionic acid, gluconolactone, and gluconic acid;
wherein the level of expression of said modified genes is eliminated or reduced or the level of activity of modified enzymes encoded by modified genes is reduced or eliminated, compared to the endogenous level of expression or activity in a parent fungus lacking one or more of said modified genes.

73. The method of claim 72, wherein the modified genes encoding enzymes responsible for the production of one or more products selected from cellobionolactone, cellobionic acid, gluconolactone, and gluconic acid are selected from the group consisting of cellobiose dehydrogenases (CDH), glucooligosaccharide dehydrogenases, glucose dehydrogenases, glucooligosaccharide oxidases, cellobiose oxidases, glucose oxidases and copper-dependent polysaccharide monooxygenases.

74. The method of claim 73, wherein the level or activity of one or more enzymes responsible for the production of one or more products selected from cellobionolactone, cellobionic acid, gluconolactone, gluconic acid
is eliminated or reduced by total or partial inactivation of at least one of said enzymes.

75. The method of claim 73, wherein at wherein the level or activity of one or more enzymes responsible for the production of one or more products selected from cellobionolactone, cellobionic acid, gluconolactone, gluconic acid
is eliminated or reduced by total or partial inactivation of at least two of said enzymes.

76. The method of claim 72, wherein the modified fungus is a M. thermophila C1 fungus and derivatives thereof.

77. The method of claim 76, wherein the modified fungus is a M. thermophila C1 fungus selected from Garg 27K, (Accession No. VKM F-3500 D) UV13-6 (Accession No. VKM F-3632 D); NG7C-19 (Accession No. VKM F-3633 D); UV18-25 (Accession No. VKM F-3631 D); strain W1L (Accession No. CBS122189) or W1L#100L (Accession No. CBS122190).

78. The method of claim 76, wherein the modified fungus is a M. thermophila C1 fungus derived from UV18-25 (Accession No. VKM F-3631 D).

79. The method of claim 76, where in modified fungus comprises a cdh1 gene disruption.

80. The method of claim 79, wherein all or part of the cdh1 gene was deleted by replacing it with a gene encoding a selectable marker.

81. The method of claim 76, wherein the modified fungus contains a cdh1 gene disruption and a cdh2 gene disruption.


SEQUENCE LISTING (52 sequences)

SEQ ID NO: 1 - DNA, 21 bp, Myceliophthora thermophila
cacaagcactgcgagtaccac

SEQ ID NO: 2 - DNA, 37 bp, Myceliophthora thermophila
gtcgagcttcattttttcgaagcgcagcaacttcaag

SEQ ID NO: 3 - DNA, 40 bp, Myceliophthora thermophila
cttgaagttgctgcgcttcgaactacctagtttgtgtgtg

SEQ ID NO: 4 - DNA, 20 bp, Myceliophthora thermophila
caccgttctccgcttctcac

SEQ ID NO: 5 - DNA, 3923 bp, Myceliophthora thermophila
cgaatagtgcttagcgacacccgtgccgccagtcgggccggaggaaccggaggatgtggtggacgaggaggggaccggggtggacgagctgacgggcgggttggggttgccgctgccgccgtcgggcaggccggagacggtggagccgatggggccgaagcggatgttggagaagatgacgttggagttgggggcctcggcctcgacctcagcggggacgcccgaggtggtggggcaggcaccgcgctcggcgcccggcttgccggcgccgtcgatgggccaggtggagtcgagccagagcatgttgacggcgtggtcgtcccagatggacatgacgaggaccatgggccccgcgagggccttgcccatctggaccatgccgcccttgtcctggaagtcggtcacgtcgccgaaggcggccttctggcggtcgcaccagtcctgggtgatggagttgccctcgacgcccgggatggtggactcggagttggggatgaccttgccgttctggacgtagaaccgcttgatctcggagagctcgccggccgagttcttgaggaactgggtgacgaccgtgatcttcttggtcgtggtacctctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagatagtgcttagcgacacccgtgccgccagtcgggccggaggaaccggaggatgtggtggacgaggaggggaccggggtggacgagctgacgggcgggttggggttgccgctgccgccgtcgggcaggccggagacggtggagccgatggggccgaagcggatgttggagaagatgacgttggagttgggggcctcggcctcgacctcagcggggacgcccgaggtggtggggcaggcaccgcgctcggcgcccggcttgccggcgccgtcgatgggccaggtggagtcgagccagagcatgttgacggcgtggtcgtcccagatggacatgacgaggaccatgggccccgcgagggccttgcccatctggaccatgccgcccttgtcctggaagtcggtcacgtcgccgaaggcggccttctggcggtcgcaccagtcctgggtgatggagttgccctcgacgcccgggatggtggactcggagttggggatgaccttgccgttctggacgtagaaccgcttgatctcggagagctcgccggccgagttcttgaggaactgggtgacgaccgtgatcttcttggtcgtggtacctt

SEQ ID NO: 6 - DNA, 20 bp, Myceliophthora thermophila
caacacgagacccgagatgg

SEQ ID NO: 7 - DNA, 46 bp, Myceliophthora thermophila
cattggttggtacgtgagggttcgaaccataagagcggaggtcagg

SEQ ID NO: 8 - DNA, 46 bp, Myceliophthora thermophila
cctgacctccgctcttatggttcgaattagaggtcttgttgggcct

SEQ ID NO: 9 - DNA, 20 bp, Myceliophthora thermophila
gagcggctttggcaattgag

SEQ ID NO: 10 - protein, 828 aa, Myceliophthora thermophila
MRTSSRLIGALAAALLPSALAQNNAPVTFTDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQLNSDAASPSYTEWAAQATKTVTGDCGGPTETSVVGVPVPTGVSFDYIVVGGGAGGIPAADKLSEAGKSVLLIEKGFASTANTGGTLGPEWLEGHDLTRFDVPGLCNQIWVDSKGIACEDTDQMAGCVLGGGTAVNAGLWFKPYSLDWDYLFPSGWKYKDVQPAINRALSRIPGTDAPSTDGKRYYQQGFDVLSKGLAGGGWTSVTANNAPDKKNRTFSHAPFMFAGGERNGPLGTYFQTAKKRSNFKLWLNTSVKRVIRQGGHITGVEVEPFRDGGYQGIVPVTKVTGRVILSAGTFGSAKILLRSGIGPNDQLQVVAASEKDGPTMISNSSWINLPVGYNLDDHLNTDTVISHPDVVFYDFYEAWDNPIQSDKDSYLNSRTGILAQAAPNIGPMFWEEIKGADGIVRQLQWTARVEGSLGAPNGKTMTMSQYLGRGATSRGRMTITPSLTTVVSDVPYLKDPNDKEAVIQGIINLQNALKNVANLTWLFPNSTITPRQYVDSMVVSPSNRRSNHWMGTNKIGTDDGRKGGSAVVDLNTKVYGTDNLFVIDASIFPGVPTTNPTSYIVTASEHASARILALPDLTPVPKYGQCGGREWSGSFVCADGSTCQMQNEWYSQCL

SEQ ID NO: 11 - protein, 787 aa, Myceliophthora thermophila
MKLLSRVGATALAATLSLQQCAAQMTEGTYTDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVLEGATSDSYEEWAKLATTTPETTCDGTGPGDKECVPAPEDTYDYIVVGAGAGGITVADKLSEAGHKVLLIEKGPPSTGLWNGTMKPEWLESTDLTRFDVPGLCNQIWVDSAGIACTDTDQMAGCVLGGGTAVNAGLWWKPHPADWDENFPEGWKSSDLADATERVFKRIPGTSHPSQDGKLYRQEGFEVISKGLANAGWKEISANEAPSEKNHTYAHTEFMFSGGERGGPLATYLASAAERSNFNLWLNTAVRRAVRSGSKVTGVELECLTDGGFSGTVNLNEGGGVIFSAGAFGSAKLLLRSGIGPEDQLEIVASSKDGETFTPKDEWINLPVGHNLIDHLNTDLIITHPDVVFYDFYAAWDEPITEDKEAYLNSRSGILAQAAPNIGPMMWDQVTPSDGITRQFQWTCRVEGDSSKTNSTHAMTLSQYLGRGVVSRGRMGITSGLSTTVAEHPYLHNNGDLEAVIQGIQNVVDALSQVADLEWVLPPPDGTVADYVNSLIVSPANRRANHWMGTAKLGTDDGRSGGTSVVDLDTKVYGTDNLFVVDASVFPGMSTGNPSAMIVIVAEQAAQRILALRS

SEQ ID NO: 12 - protein, 577 aa, Myceliophthora thermophila
MQVASKLVAVTGGALALWLHPVAAQEGCTNISSTETYDYIVVGSGAGGIPVADRLSEAGHKVLLIEKGPPSTGRWGGIMKPEWLIGTNLTRFDVPGLCNQIWADPTGAICTDVDQMAGCMLGGGTAVNAGLWWKPHPADWDVNFPEGWHSEDMAEATERVFERIPGTITPSMDGKRYLSQGFDMLGGSLEAAGWEYLVPNEHPDRKNRTYGHSTFMYSGGERGGPLATYLVSAVQREGFTLWMNTTVTRIIREGGHATGVEVQCSNSEAGQAGIVPLTPKTGRVIVSAGAFGSAKLLFRSGIGPKDQLNIVKNSTDGPSMISEDQWIELPVGYNLNDHVGTDIEIAHPDVVFYDYYGAWDEPIVEDTERYVANRTGPLAQAAPNIGPIFWETIKGSDGVSRHLQWQARVEGKLNTSMTITQYLGTGSRSRGRMTITRRLNTVVSTPPYLRDEYDREAVIQGIANLRESLKGVANLTWITPPSNVTVEDFVDSIPATPARRCSNHWIGTAKIGLDDGREGGTSVVDLNTKVYGTDNIFVVDASIFPGHITGNPSAAIVIAAEYAAAKILALPAPEDAA

SEQ ID NO: 13 - protein, 497 aa, Myceliophthora thermophila
MHLLPLTVSATAVVSAASSPHAKRAAIDECLKNAKVPVTARNSTEWKTDASPFNDRLPYTPAAIAKPATVEHIQAAVLCAAEVGVKANPKSGGHSYASFGLGGEDGHLVVELDRMYNVTLDPETHIATVQPGARLGHIATVLYEEGKRAFSHGTCPGVGVGGHSLHGGFGFSSHSHGLAVDWITSADVVLANGSLVTASETENPDLFWALRGAGSNFGIVASFRFKTFAAPPNVTSYEINLPWTNSSNVVKGWGALQEWLLNGGMPEEMNMRVLGNAFQTQLQGLYHGNASALKTAIQPLLALLDANLSSVQEHDWMEGFRHYAYSGEIDITDPGYDQSETFYSKSLVTSALPPDVLERVAEYWIETANKVRRSWYIIIDMYGGPNSAVTRVPPGAGSYAFRDPERHLFLYELYDRSFGPYPDDGFAFLDGWVHAFTGGLDSSDWGMYINYADPGLDRAEAQEVYYRQNLDRLRRIKQQLDPTELFYYPQAVEPAEV

SEQ ID NO: 14 - protein, 500 aa, Myceliophthora thermophila
MHPLGGSLRLGGLLGLAVASQGSRLHHRFGGGGNQTDRLTECLTEAGVPVDMAGTAEYAIDVSSFNLRLNYTPAAVAAAGTADHVRDAVACAARLGVKATAKCGGHSYASFGLGGEDGHLVIEMSRMNRVVLDDETGIATVEGGARLGHLAVELWDQGKRAISHGTCPGVGVGGHVLHGGYGMSSHTHGLALDWMVGATVVLANASVVECSETENPDLFWALRGAGSSMGVVTEFRFKTFEPPENLTYFVATAQWPTEDRALAGLAAVQEYAKTMPAELNMRLYIANRFVNLEGLYYGDDAALHHTLAPLLDQANATLALAQTGGWLDQLKHFGGSNLDQGHGHEEHETFHSTSLYTGPLDEDRLRAFVGYWFGPAKNNTRSWHVQIDLHGGENSAVSVAPRSTAYAHRDSLLMFLLYDRADRGEFPADGAALMDNFAAAVTAGFDPDDWGMYVNYPNPSLSQDDAQARYWGPNLARLRAIKKDVDPDDLFHYPQGILPA

SEQ ID NO: 15 - protein, 303 aa, Myceliophthora thermophila
MKGLLGAAALSLAVSDVSAHYIFQQLTTGGVKHAVYQYIRKNTNYNSPVTDLTSNDLRCNVGATGAGTDTVTVRAGDSFTFTTDTPVYHQGPTSIYMSKAPGSASDYDGSGGWFKIKDWGADFSSGQATWTLASDYTATIPECIPPGDYLLRIQQLGIHNPWPAGIPQFYISCAQITVTGGGSANPGPTVSIPGAFKETDPGYTVNIYNNFHNYTVPGPAVFTCNGSGGNNGGGSNPVTTTTTTTTRPSTSTAQSQPSSSPTSPSSCTVAKWGQCGGQGYSGCTVCAAGSTCQKTNDYYSQCL

SEQ ID NO: 16 - protein, 241 aa, Myceliophthora thermophila
MKTLAALVVSAALVAAHGYVDHATIGGKDYQFYQPYQDPYMGDNKPDRVSRSIPGNGPVEDVNSIDLQCHAGAEPAKLHAPAAAGSTVTLYWTLWPDSHVGPVITYMARCPDTGCQDWSPGTKPVWFKIKEGGREGTSNVWAATPLMTAPSAYTYTIPSCLKSGYYLVRHEIIALHSAWQYPGAQFYPGCHQLQVTGGGSTVPSTNLVSFPGAYKGSDPGITYDAYKAQPYTIPGPAVFTC

SEQ ID NO: 17 - protein, 225 aa, Myceliophthora thermophila
MRYFLQLAAAAAFAVNSAAGHYIFQQFATGGSKYPPWKYIRRNTNPDWLQNGPVTDLSSTDLRCNVGGQVSNGTETITLNAGDEFSFILDTPVYHAGPTSLYMSKAPGAVADYDGGGAWFKIYDWGPSGTSWTLSGTYTQRIPKCIPDGEYLLRIQQIGLHNPGAAPQFYISCAQVKVVDGGSTNPTPTAQIPGAFHSNDPGLTVNIYNDPLTNYVVPGPRVSHW

SEQ ID NO: 18 - protein, 286 aa, Myceliophthora thermophila
MHPSLLFTLGLASVLVPLSSAHTTFTTLFVNDVNQGDGTCIRMAKKGNVATHPLAGGLDSEDMACGRDGQEPVAFTCPAPAGAKLTLEFRMWADASQSGSIDPSHLGVMAIYLKKVSDMKSDAAAGPGWFKIWDQGYDLAAKKWATEKLIDNNGLLSVNLPTGLPTGYYLARQEIITLQNVTNDRPEPQFYVGCAQLYVEGTSDSPIPSDKTVSIPGHISDPADPGLTFNVYTGDASTYKPPGPEAGAGAASTPTFAAPGAAKTPQPNSERARRREAHWRRLESAE

SEQ ID NO: 19 - protein, 242 aa, Myceliophthora thermophila
MKSSTPALFAAGLLAQHAAAHSIFQQASSGSTDFDTLCTRMPPNNSPVTSVTSGDMTCNVGGTKGVSGFCEVNAGDEFTVEMHAQPGDRSCANEAIGGNHFGPVLIYMSKVDDASTADGSGDWFKVDEFGYDASTKTWGTDKLNENCGKRTFNIPSHIPAGDYLVRAEAIALHTANQPGGAQFYMSCYQVRISGGEGGQLPAGVKIPGAYSANDPGILVDIWGNDFNEYVIPGPPVIDSSYF

SEQ ID NO: 20 - protein, 255 aa, Myceliophthora thermophila
MYRTLGSIALLAGGAAAHGAVTSYNIAGKDYPGYSGFAPTGQDVIQWQWPDYNPVLSASDPKLRCNGGTGAALYAEAAPGDTITATWAQWTHSQGPILVWMYKCPGDFSSCDGSGAGWFKIDEAGFHGDGTTVFLDTETPSGWDIAKLVGGNKSWSSKIPDGLAPGNYLVRHELIALHQANNPQFYPECAQIKVTGSGTAEPAASYKAAIPGYCQQSDPNISFNINDHSLPQEYKIPGPPVFKGTASAKARAFQA

SEQ ID NO: 21 - protein, 237 aa, Myceliophthora thermophila
MKVLAPLILAGAASAHTIFSSLEVGGVNQGIGQGVRVPSYNGPIEDVTSNSIACNGPPNPTTPTNKVITVRAGETVTAVWRYMLSTTGSAPNDIMDSSHKGPTMAYLKKVDNATTDSGVGGGWFKIQEDGLTNGVWGTERVINGQGRHNIKIPECIAPGQYLLRAEMLALHGASNYPGAQFYMECAQLNIVGGTGSKTPSTVSFPGAYKGTDPGVKINIYWPPVTSYQIPGPGVFTC

SEQ ID NO: 22 - protein, 245 aa, Myceliophthora thermophila
MLLLTLATLVTLLARHVSAHARLFRVSVDGKDQGDGLNKYIRSPATNDPVRDLSSAAIVCNTQGSKAAPDFVRAAAGDKLTFLWAHDNPDDPVDYVLDPSHKGAILTYVAAYPSGDPTGPIWSKLAEEGFTGGQWATIKMIDNGGKVDVTLPEALAPGKYLIRQELLALHRADFACDDPAHPNRGAESYPNCVQVEVSGSGDKKPDQNFDFNKGYTCDNKGLHFKIYIGQDSQYVAPGPRPWNGS

SEQ ID NO: 23 - protein, 342 aa, Myceliophthora thermophila
MSKASALLAGLTGAALVAAHGHVSHIVVNGVYYRNYDPTTDWYQPNPPTVIGWTAADQDNGFVEPNSFGTPDIICHKSATPGGGHATVAAGDKINIVWTPEWPESHIGPVIDYLAACNGDCETVDKSSLRWFKIDGAGYDKAAGRWAADALRANGNSWLVQIPSDLKAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 24 - protein, 306 aa, Myceliophthora thermophila
MKPFSLVALATAVSGHAIFQRVSVNGQDQGQLKGVRAPSSNSPIQNVNDANMACNANIVYHDNTIIKVPAGARVGAWWQHVIGGPQGANDPDNPIAASHKGPIQVYLAKVDNAATASPSGLKWFKVAERGLNNGVWAVDELIANNGWHYFDLPSCVAPGQYLMRVELLALHSASSPGGAQFYMGCAQIEVTGSGTNSGSDFVSFPGAYSANDPGILLSIYDSSGKPNNGGRSYPIPGPRPISCSGSGGGGNNGGDGGDDNNGGGNNNGGGSVPLYGQCGGIGYTGPTTCAQGTCKVSNEYYSQCLP

SEQ ID NO: 25 - protein, 246 aa, Myceliophthora thermophila
MKLSLFSVLATALTVEGHAIFQKVSVNGADQGSLTGLRAPNNNNPVQNVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPASTVSFPGAYSASDPGILINIYGATGQPDNNGQPYTAPGPAPISC

SEQ ID NO: 26 - protein, 306 aa, Myceliophthora thermophila
MPPPRLSTLLPLLALIAPTALGHSHLGYIIINGEVYQGFDPRPEQANSPLRVGWSTGAIDDGFVAPANYSSPDIICHIEGASPPAHAPVRAGDRVHVQWNGWPLGHVGPVLSYLAPCGGLEGSESGCAGVDKRQLRWTKVDDSLPAMELRWATDVLIAANNSWQVEIPRGLRDGPYVLRHEIVALHYAAEPGGAQNYPLCVNLWVEGGDGSMELDHFDATQFYRPDDPGILLNVTAGLRSYAVPGPTLAAGATPVPYAQQNISSARADGTPVIVTRSTETVPFTAAPTPAETAEAKGGRYGRNFRG

SEQ ID NO: 27 - protein, 323 aa, Myceliophthora thermophila
MKSFTLTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGQGYTGCTNCASGSTCSAVSPPYYSQCV

SEQ ID NO: 28 - protein, 346 aa, Myceliophthora thermophila
MSSFTSKGLLSALMGAATVAAHGHVTNIVINGVSYQNFDPFTHPYMQNPPTVVGWTASNTDNGFVGPESFSSPDIICHKSATNAGGHAVVAAGDKVFIQWDTWPESHHGPVIDYLADCGDAGCEKVDKTTLKFFKISESGLLDGTNAPGKWASDTLIANNNSWLVQIPPNIAPGNYVLRHEIIALHSAGQQNGAQNYPQCFNLQVTGSGTQKPSGVLGTELYKATDAGILANIYTSPVTYQIPGPAIISGASAVQQTTSAITASASAITGSATAAPTAATTTAAAAATTTTTAGSGATATPSTGGSPSSAQPAPTTAAATSSPARPTRCAGLKKRRRHARDVKVAL

SEQ ID NO: 29 - protein, 216 aa, Myceliophthora thermophila
MKLATLLAALTLGVADQQLSVGSRKFGVYEHIRKNTNYNSPVTDLSDTNLRCNVGGGSGTSTTVLDVKAGDSFTFFSDVAVYHQGPISLYMSKTPGSAQDYDGSGDCFKIYDWGPTFNGGQASWPTRNSYEYSILKCIRDGEYLLRIQSLAIHNPGALPQFYISCAQVNVTGGGTVTPRSRRPILGASRDRSLTERKQIYFNFHSYIVPGPAVFKC

SEQ ID NO: 30 - protein, 230 aa, Myceliophthora thermophila
MRLLASLLLAATAVQAHYTFPRLVVNGQPEESDWSATRMTKNAQSKQGVENPTSGDIRCYTSQTAANVVTVPAGSTIHYISTQQINHPGPTQYYLAKVPPGSSAKTFDGSGAVWFKISTTMPTVDSNKQMFWPGQNTYETSNTTIPANTPDGEYLLRVKQIALHMASQPNKVQFYLACTQIKITGGRNGTPSPLVALPGAYKSTDPGILVDIYSMKPESYQPPGPPVWRG

SEQ ID NO: 31 - protein, 225 aa, Myceliophthora thermophila
MLTTTFALLTAALGVSAHYTLPRVGTGSDWQHVRRADNWQNNGFVGDVNSEQIRCFQATPAGAQDVYTVQAGSTVTYHANPSIYHPGPMQFYLARVPDGQDVKSWTGEGAVWFKVYEEQPQFGAQLTWPSNGKSSFEVPIPSCIRAGNYLLRAEHIALHVAQSQGGAQFYISCAQLQVTGGGSTEPSQKVSFPGAYKSTDPGILININYPVPTSYQNPGPAVFRC

SEQ ID NO: 32 - protein, 254 aa, Myceliophthora thermophila
MALQLLASLALLSVPALAHGGLANYTVGDTWYRGYDPNLPPETQLNQTWMIQRQWATIDPVFTVSEPYLACNNPGAPPPSYIPIRAGDKITAVYWYWLHAIGPMSVWLARCGDTPAADCRDVDVNRVGWFKIWEGGLLEGPNLAEGLWYQKDFQRWDGSPSLWPVTIPKGLKSGTYIIRHEILSLHVALKPQFYPECAHLNITGGGDLLPPEETLVRFPGVYKEDDPSIFIDVYSEENANRTDYTVPGGPIWEG

SEQ ID NO: 33 - protein, 227 aa, Myceliophthora thermophila
MKLSAAIAVLAAALAEGHYTFPSIANTADWQYVRITTNFQSNGPVTDVNSDQIRCYERNPGTGAPGIYNVTAGTTINYNAKSSISHPGPMAFYIAKVPAGQSAATWDGKGAVWSKIHQEMPHFGTSLTWDSNGRTSMPVTIPRCLQDGEYLLRAEHIALHSAGSPGGAQFYISCAQLSVTGGSGTWNPRNKVSFPGAYKATDPGILINIYYPVPTSYTPAGPPVDTC

SEQ ID NO: 34 - protein, 235 aa, Myceliophthora thermophila
MKALSLLAAAGAVSAHTIFVQLEADGTRYPVSYGIRDPTYDGPITDVTSNDVACNGGPNPTTPSSDVITVTAGTTVKAIWRHTLQSGPDDVMDASHKGPTLAYIKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 35 - protein, 232 aa, Myceliophthora thermophila
MKLTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIEDGEYLLRVEHIALHSASSVGGAQFYIACAQLSVTGGSGTLNTGSLVSLPGAYKATDPGILFQLYWPIPTEYINPGPAPVSC

SEQ ID NO: 36 - protein, 193 aa, Myceliophthora thermophila
MRSTLAGALAAIAAQKVAGHATFQQLWVDGTDYISFHSLCSVLVPFLPDPKLMTDGPSTAPPVSAFRLATHPSPMWEAETSSATLAPAPRGSTEGQAVTHSSQQPGDRSCNNEAIGGAHWGPVQVYLTKVQDAATADGSTGWFKIFSDSWSKKPGGNLGDSGVPLTDRITVLQAGFTCKAVSPPYYSQCAPSS

SEQ ID NO: 37 - protein, 278 aa, Myceliophthora thermophila
MFSLKFFILAGGLAVLTEAHIRLVSPAPFTNPDQGPSPLLEAGSDYPCHNGNGGGYQGTPTQMAKGSKQQLAFQGSAVHGGGSCQVSITYDENPTAQSSFKVIHSIQGGCPARAETIPDCSAQNINACNIKPDNAQMDTPDKYEFTIPEDLPSGKATLAWTWINTIGNREFYMACAPVEITGDGGSESALAALPDMVIANIPSIGGTCATEEGKYYEYPNPGKSVETIPGWTDLVPLQGECGAASGVSGSGGNASSATPAAGAAPTPAVRGRRPTWNA

SEQ ID NO: 38 - protein, 405 aa, Myceliophthora thermophila
MTTLYTTTAAALALLLGSAQGHMIMNTPTPYNYHGDNAVQVNPLGPGFPFPCQGRTDHIIETTTIQAGGSQVVKFTGSAVHGGGSCQFSVTYENPPPADKSKWKTIYTIIGGCPASAQGNLQSTGKDEDGREDGNDCGNDTGTECVRQFNVPIPKELPSGNATFAWTWFNKIGNREIYMNCAPVQITGGADDDKFLQELPDLFVANVDGECTTGNGVFNIPNPGKYGKVLEDPTQGSEGSCTKADGIPKFDDSGSGSGSGSGSGSGSGSDGGSSSASPTETPAASNTGLPGGIFMTTAPSPSPSSGSASGSGSGSGSGSGSGSGSGSGSDSGSGSGSGSGSGSGSGSGSGASGGQACSENGAIICFSPTSFGLCANGIAIPQPVAAGTTCNNGVIARRSAKFIYW

SEQ ID NO: 39 - protein, 418 aa, Myceliophthora thermophila
MFTYGLATGALALLAGSQLAAAHAQVEFPAPFRSKFNPNVDSSMIDYSYTSPLDPSGANYPCKGYHTDLGTPAGKPTATFAPGSEYQFKMAAGGARHGGGSCQVSLSYDKGKTFTVIKSIIGGCPLEDSYSFTIPADAPEGEAIWAWTWSNEIGNREHYMNCAPVIIGSDGGSSSSKKREVAERADTAFSSRPPVFAANIGNGCTTVEGVDVDYPQPGPDVVRSGDKIGPPSGNCGPTSGSGSGSGSGSDSGSGSGSGSGSGSGSGSSSPTTTSAAQVTSAPAAPTTSSAAGGLPGGVFITPSSPSETTLSTKTSAAVLPTGTGTGSGSGSGSGSGSGSGNTAGAQAQGTPCTEEGAWNCIGGTQFQRCASGVWSAPQPVSAGTVCKTGQSGSLTIEAATSKLRVRRVRRGSAKVRLA

SEQ ID NO: 40 - protein, 436 aa, Myceliophthora thermophila
MPSVFSLTGGVAMLLIGGASGHMIMNSPTPYNLNIQPLLQVDPLSGDSYPFPCQNQYGFTTRTLVEAGGSTLVNFTGGGQHGGGSCQFSITYDEPVNGGDWNKSAKFKTIYSIIGGCPAVFTDETHNLAPVAMDKNMRQDSQHCGNDSGIDCIRQFMVPIPKFLKNGPATFAWTWFNKLGNKEMYIITGGTGDEKEMEKLPDIFIANYPNDPEVPNCITGTRADKVVVNFPNPANTIPAAQSVPVFASVPVSQDGCPASCSTRSSSPTYHHQSSTTAIETHTNEPISGSPGSVPRSSATPPPPPPWMVLPSTLTTEVAAWTSIDLGTVTVLPQPRPTADKPDDQAVACAAHGELVCFDEDTYGLCNWGWAVPQRMAAGTKCEDGKVVKREHGGEDRKHGGGHGWEARKLMRPVVLVMEEEDGMVWIPKDDEVLQSQ

SEQ ID NO: 41 - protein, 320 aa, Myceliophthora thermophila
MALRLSLLQVFAAAILASFSLLLAHGESAATVRSCTTHDHHNTLAELFPNSATGVLNATLAIIPISLETARRLIPPQYGILERAYRALVPSFPEGMYPLMVQAAHDHDVQLRAYGITIDDFSRVGFEFPFLDLAGDGYSSFRWAPAQLISATNPIALEGSRAYGTLVTPAKYEPLCDAYHRLANGHTYFRGVGLDSSDLFELEMARLSHAALNPYPLELFKNITNQPTFANATSCDNMIRLFNTSMTVGANAPVHVHGRVRARAFPFQEAEKEWTDIYGVQVATPFIENNYLECRSMRGYSGTGGPGDSYVHEANVNDEL

SEQ ID NO: 42 - protein, 574 aa, Myceliophthora thermophila
MPASLLRFLALAGTAVGLTTNHNHSPSCRVLPGDAAWPSSRDWAKLNKTLNGHLIATVPQASVCHKSPFGQYDAQACEELKSSWDISTITHVNAPGDVLSQNFQNYSCVPFTDPSQPCQLGNYPSYVVNVTGAADVQAALKFAQKHNVRIVIKNTGHDYLGKSTGKGALSLWMHNLKSTKFIKNYKAPYYKGPAAKLGAGVEGFEAYAMANSTGHRIVGGTCPTVGIVGGYTQGGGHSILSSSYGVAADNVLEWEVVTADGRHLVATPTRNSDLYWALSGGGGGTFAVVLSMTARLHRDGIVGGTLLGFNDSAVGNEVYWEAVAAFHALLPDFLDGGNSFTYSVGNNSLTAYGTMPGADRDAVDRLLRPFLDDLASRGITPVVQPRVSTNYYDHFFTYLGPAPYGNAAYFPFTNSRIIPRSLVTDPKSNAVVTDLFRNISQVPAFSPFYCDSFSVADKPHPANSLHPAWRTGMLLCAPAGSWDWDASPEEMAARDRYAAETLQPMMDAATPGGSVYLNEANHLYANWKESFYGDNYARLLRVKKKYDPDSVFYVKTGVGSEVWDVDATGRLCRA

SEQ ID NO: 43 - protein, 581 aa, Myceliophthora thermophila
MARVLSTGALLALLNFATPGLGAAHVSNCRYIPGDKGWPSQSDWAKLNRTVGGRLIATVPQAHVCHAGGPGVNQAACDALKDPLVFEKTAPAYVNKPAEIINAYWQNRSCDPFSKTSRPCVLGNYPVYSINVSGAHDAIAGLDFARRQNIRLSIQNTGHDYNGRSAGFGSLSLWMHNLQGIEVIPRYRSRAYSGPAVKLGAGVHAGVALQYLGQRGYRLVTGECGTVGVAGGYSMGGGHGPLNGAYGMASDNVLEWEVVTGDGRHIVATPEKNSDIYWAMSGGGGGSYGVALSMTARIYRDGPVLGPVLTFTAPDVGNETYWGAVDAFLKRLPGMLRGTSSSIQFSFWNNQFGALFVMPDERNSSAADAKLAPLLRDLDAIGIPYDLTVSQSETFLDYYSSWYGPLPFGYEPPSTTLNSRLVPVRVAQDDQARRQLIDAMRLTTQTGEFTVGCSAADVGSVRHPDNAVLPAWRKSVAICNVNAFWNWTAPLEQNLEVKRRMVDVYSPAWDAATPGSGVYLNEIDPWYRSDFKVNMFGSNYRRLLSIKHKYDPYHLFYGHNLVGSDDFSIDGAGRLCYSSGR

SEQ ID NO: 44 - protein, 638 aa, Myceliophthora thermophila
MTSSFSLRSAALATVSALFAERTIAGAPADKCPTRFAWEDAQLTDEVISSTDPLLFGFGYPVDTAASASENGGPELPNCKAMPGDASWPSPETWALFNSTLGGALIETVPIAAPCYNNWPQRDAAACEYVTEHWGDPRMHVEDPTSAMFPLYQGRTCLPTDDPDGSNCTLGGYAAYSVAATNVSQIQLALNFARNTNLRLVVRNTGHDFNDRSIGAGALSVWTHKLRDIQFLPDYNCRGYSGPAFKLGSGIMTEELYAAAEENNVTVVGGECRTVGIAGGYIAGGGHSPMSALLGMGADQVLSLEVVLPNGRFVTANEDTNPDLYWALRGAGGSTYGVVTSVTIRAYPKIPNTLMTYSYTTSPNVTTDTFFASLGVYMSYFDRITAAGAYGYFIVVSIGPDQYLFSMMPLWGANMTKPQLTELATPLLNDLAGLGIAITPNVTEYPSMFSAFNGAFPAAEQVGAYDNHAASRIFPKESFEPDRLNDTLAAVRHAVEGGGVLVGYNIRAAPNPAVNQTNSVHPAWRRTTGFFILGASWPANATDAQIQQASETLTNDWMARWRAVSPGGGSYASEGDINEPDFQQSFYGDHYPRLLELKKKYDPTGLFYAPTAVGSEYWYITDQLPWIPTQNGRLCRKK

SEQ ID NO: 45 - protein, 643 aa, Myceliophthora thermophila
MAVLSLQRALLLAGAALPLVQQGFAQTIKTEDGEVLPANEVTVAPAAEPASEDDEIAALQLTDNVLANLTAHELTDVELFQFGDDADAADPTQIAKRTAVGSCKTYPGDWKWPSRLTWSVFNLLTGGALIETVPIGAVCYPNSGVYNAAKCADIIEHWQESATHEADPTSVMSPLFQGETCMPQNGNTSQCTLGGFPAYAVKATSVYQIQLAVNFARSLNLRLVVKNTGHDFLGKSLGYGSLSIWTHHLKSIRFRQSVRTPSYSGPALELGAGVTVGELYAAANQYGVTAVGGECKGVGVAGGYLAGGGHSPLSGKYGLGSDQVLSIDLVLPNGRFVTASETENTDLFWALRGGGGSTFGVVTSVTVKAHPKMKFSGAMYAYWSKFPEYADQEVYGYGNIFPRGAPGSGYTWTMLPWMVPNKTLSEFKAMVQPLFDEWTAMGFEFEPQYFEHDNFYDAWTSHFPTEVVANSNLRTASRLFPRSAWDDDTTRIAMFDAVRSVIEEGSALIQYNMNPAAPAGTPASGANSHWRDAVWFGIMGTGWAPGISQEELEAVQRKITDDWMGRLRPYGPGGYGNEGDVMEPDFAEAFFGTNYDRLLQIKRTVDPYDLFWAPTAVGSERWKIAGQPDWLTLQTGKLCKVSN

SEQ ID NO: 46 - protein, 594 aa, Myceliophthora thermophila
MRGGVFGLLCGALLLPGSKSTKWVLRRAGDSDYACKCYPGDNCWPNKGQWQKLNNTVGGNLRVNIPPGAPCYNTFQGLLGNVLQTYNAAGCQKVRQNWGDEQFQIEMPAAGLWTYFTNDTCRPTTNPTDTCTPGYYPVLVIMAKTTAHIQAGINFARENNLRLIIRNTGHDFLGRSVGWGALVINTHSFQDIKFTDSWEGAGNYTGPAVTVGAGVQAFQVLKKANALNPPKIMVTGECATVGVAGGLVQGGGHGPLTGFYGFVADNALEFKVITADGKLNTANAETNADLFWALRGGGPAAFAVIVEASYRIFDDKPTAGVLLDIDQTHMTNATLFWEAVRVFHSYSTHFVDNDLYVYYELGTAGQNLHVHPIVGVGKTPDELQAVLQPMFDDLDALGISYYTTGAADFPTFYDLYQAMFETEVAGNSALTGGWTIARQDAEERHEAIISAFQTVVRAGSFMIGHMWSAGHGLPEERWAESSVNPRFRSVVDKLITVVPVAGNAPLADKAAAQDTLTNVVDAALREASPNGCAYVNEADPYEPNWQQAFWGDNYPRLLEIRKKYDPDGVFYAISTPGTENWEQIETGTRLCRKL

SEQ ID NO: 47 - protein, 601 aa, Myceliophthora thermophila
MSPPRGLVSALQLLSSVLAVAAGLTPSLRSDIEHSSHPRCKAVPGSPGWPSTREWNRLNESIAGRLLRPTPPGAVCHSGQGDGESPECAAVREQWSTYEFHQADPVSVDWNNWANDTCLPFPGAPCSGQGYPVFVINATEARHVQLGVQFAKKHNIRLVVKSTGHDYVGRSVAPNSLSIWTHYMRDIKTHKSFRPKRCKATIDSTAVTVGAGTQMWDLYNALDLLNQTVVGGGSKTVSVGGYVTGAGHGLLSPTYGLAADQVLEMELVTPNGDIVTANECQNEDLFWAMRGGGGSTFGVLTSVTMKTFATPRIEAATVMLMTTDVAQPRPIFDMVAYVLSQFPSLADRGLSGYSYVIRETPNPLDNGTTSVGGIVFAGVVQNSSPEGMRKLWDPVLARVNATWPGRFVHIYEPSSYPTFLSWFSEHFDSDEAGHDVILGSRLLDRAALTANLTALSAAYDRFTAGATSTAYLVSGRGVHHARPRGPRGNAVLPAWRSAYVHATLGEGFPPLNATAAAAAKERVRERVAALRELAPRMGAYVNEANPEEPNWQKEFWGSNYKRLHAIKRAVDPDDVLWCTPCVGNERWEQVGDRLCRVERSH

SEQ ID NO: 48 - protein, 272 aa, Myceliophthora thermophila
MLPWRRVLVLAPFVANQSCDPFTSPSKPCTLGNYVVYAVEAQSAADIIAAVKFAKQNNIRFVIRNTGHDYLGRSTGAGALSFREWKGKDYTGSAVKLGAGVQGFQVLSAALEKKQVVVGGECPTVGIAGGYTQGGGHSALSTSFGRRQHARVRGRHGLAHAERRNLYWALSGAMVVYYSTSSFFQIAPLTAYNKTAAEVEAMLSPFAAKLTAMGVKYTLGYSQSATYYDHYDKYFGPLPVGNIEVGIAQYGGRLVPLSTFANDPAAMSAVTR

SEQ ID NO: 49 - protein, 418 aa, Myceliophthora thermophila
MARFPLLSALICILASALHVSAAISNYTAICNEIKDRVSQQSDVTYPIQGVTFSDRIHHWFDSSTEIPACVAEVGSVEDVSLVLQIVGASRTPFAVYSGGHASNVGFSSTKGVHITLRRFNQTQLSEDKTTVTIGFGQTWVDVFEALADSGVNVVGGRVPGPGIGGFTLGGGYSCPRRSGQVPALLNATERFASENRDPRASVLTSVDGTTAVGPTALGLFFYDGPEKPEIFNLFDGLSTISDSTGRKPFLDLIRGFPAEIVYNSRGTFATFSTTGITGRFLETVRQEAENMGKIAALHGATTINYDAQPFLQYGRHATPSAFPHSDSLFPFNLYFAWRNSSEDEFWYGKIHQTLDTLKRVATEEGIYREDFPDYPNYALSGTSAEKLYGETNAGRLRQIRDQIDPDRIMDLAGGFAL

SEQ ID NO: 50 - protein, 610 aa, Myceliophthora thermophila
MPKGLGQSLAARSAVAAAAAAAAAAGAGRPACLALRTVPVRLSSTENSKQPRPSGASFQGQISNSIMQRLQRERAELERVARTRPESSMTRNFSLTFVIFFVGSVCWVLGYQYPRDADPSSTLPLNATRPPTYNLNPAHLEAAWADFVEIVGQENVSTLEDDIKQHATSEWSSHRSDPAHKPFCVVYPATTEQVAAIMKVCHTRRIPVVGYSGGTSLEGHYTPTRGGICVDFGRMDKIVALHKDDLDVVVQPGIGWEDLNEQLAEHNLFFPPDPGPGARIGGMIGTGCSGTNAYRYGTMRDWVLSLTVVLADGTVIKTRQRPRKSSAGYDLTRLFIGSEGTLGLVTEATLKLCVKPAAASVAVASFPSIRHAADCVARVVRDGVGVAAVEILDDDQMRFINAAGTTTRKWPEAPTLFFKFAGAPAGVKEQIDLVRLMAKRAGGQSFDFARDESEQAELWSARKDALFGTMAQRRPGDHVWTGDVAVPVSRLPDIIEETKRDLKASGLTSSIVGHVGDGNFHIILLYNDAERKLAEDCVHRMVKRAVEMEGTVTGEHGVGLVKRDYLPHELGETTVDAMRKIKAAFDPLCLLNCDKVVRVQKPARGEVAEW

SEQ ID NO: 51 - protein, 477 aa, Myceliophthora thermophila
MLLPSVLGALLAASCPSAPTQAHDRHNVAVSALARPISAAAVLLFFGAHPAMATPERSLAEFIDSLQLPGQQAAELTAELQKDAELVSFLEARAHSGQKSSSNSSTTTTATTTTTTTTTTPATTTGLSAACRVLQTALGSGKVVSADNETEVNATCNARIKLTAVRSWHREGRVSKTTGRQQRGRHPRIVTSFELYTIPVHEVWVEGLAFSPAQVPEVFEAYAAFQKSTTPDIKATVSVVVSLDIVLVALLYTEPAASRPQAFSPFDKLTPLSVILPPTNMTVLQFSQISAGTQPNTASRGPFQLDSLILSDTVPLPSHDYRAASSKIDAQLYTDVYNIWLERATKVKEATGANQTFTIQTFSKNLVQQGIKKGGNPLGMPLEDFQCWTTLMDWNEAADDAAVRSAAIETTEAWARLGAQRGLAVDYLYLNDASRDQNPLASYGPANVARLKAVAAKYDPDRVFQTLQNGGFLLRDV

SEQ ID NO: 52 - protein, 504 aa, Myceliophthora thermophila
MTSVASLLWVWAILTAIPFVAASTPSPADEVALVAATCEGLQRVLGEVPKEILTQIKFRSQTSWQKPSCVASPRSASEVQSIVATLGRKNVPFAVRSGGHSPAPFDANINTGVLISLGKLNHISYDKERQTAGIGPGARWGEVYATLDPFNVTVVGGRVGDVGVGGLVLGGGLSYLSNLYGLACDNVLEYKVVLANGTLVTASSTSHPDLFWALKGGANNFGIVTQFTVATYPIKEAWGGLRTYSIEQMPQVLDALHQYQTAEDKDPYANLFISIPITNATDYGILVTFVYLKPVEEPPAYAAFYNLTPTADATALTTLNDLMTNFLQPEFPRYNWYTTSFRPTPSTYSGISNLLLTAPEVGKIQALTGGTLVASFQPIDASVGALSSPSSSSTTPNQPPPPSSSSSSSYAGRQPQGDWAGNALGLRAARSIGQSQNSSARLSRAEGSFLDYVFMNDASWRQPVIAGYGPASLRRLRQVARRYDPDRVFVRLLRGGQKVPWEEE